(19) United States
(12) Patent Application Publication
(10) Pub. No.: US 2025/0270198 A1
(43) Pub. Date: Aug. 28, 2025

(54) PYRIDONE COMPOUNDS AS TRPA1 INHIBITORS

(71) Applicant: D. E. Shaw Research, LLC, New York, NY (US)

(72) Inventors: Fabrizio GIORDANETTO, New York, NY (US); Morten Østergaard JENSEN, Copenhagen (DK); Vishwanath JOGINI, Hyderabad (IN); Roger John SNOW, Danbury, CT (US)

(21) Appl. No.: 18/862,249

(22) PCT Filed: May 3, 2023
(86) PCT No.: PCT/US2023/066533

§ 371 Date: Nov. 1, 2024

Related U.S. Application Data
(60) Provisional application No. 63/338,181, filed on May 4, 2022.

(51) Int. Cl. C07D 413/06; A61K 31/4439; A61K 31/497; A61K 31/506; A61K 31/53; C07D 413/14

(52) U.S. Cl. CPC C07D 413/06; A61K 31/4439; A61K 31/497; A61K 31/506; A61K 31/53; C07D 413/14

(57) ABSTRACT

A compound of Formula (I) or a pharmaceutically acceptable salt thereof, is described, wherein the substituents are as defined herein. Pharmaceutical compositions comprising the same arid method of using the same for treatment of medical conditions, including pain, a skin disorder, a respiratory disease, a fibrotic disease, an inner ear disorder, fever or another disorder of thermoregulation, are also described.

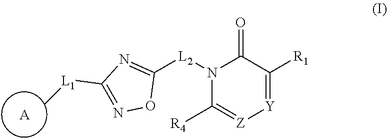

### CROSS-REFERENCE TO RELATED APPLICATIONS

This application claims the benefit and priority of U.S. Provisional Application No. 63/338,181 filed on May 4, 2022, the content of which is incorporated herein by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals as potassium channel blockers.

### BACKGROUND

Transient receptor potential channels (TRP channels) are a family of voltage-gated ion channels located primarily on the plasma membrane of mammalian cells. There are approximately 30 structurally related TRP channels subdivided into several groups: TRPA, TRPC, TRPM, TRPML, TRPN, TRPP, and TRPV. Transient receptor potential ankyrin 1 (TRPA1), a member of the TRPA subfamily, is a cation-selective, calcium-permeable ion channel (Montell, C., 2005, Sci. STKE, 272:re3).

TRPA channels are characterized structurally by the presence of multiple N-terminal ankyrin repeats forming a large intracellular domain (Montell, C., 2005, Sci. STKE, 272:re3). The human TRPA1 has approximately 14 N-terminal ankyrin repeats. The TRPA1 protein is a homotetramer. Each subunit has six transmembrane helices that form a central pore, which is surrounded by voltage-sensor-like domains. The TRPA1 protein also contains a C-terminal extension (Terrett, J. A. et al., 2021, J. Med. Chem. 64, 7, 3843-3869).

TRPA1 is highly expressed in the plasma membrane of primary sensory neurons where it functions as a polymodal sensor for exogenous and endogenous stimuli. These sensory neurons are in the dorsal root and nodose ganglia and connect with skin, lung, small intestine, colon, pancreas, skeletal muscle, heart, brain, bladder, and several immune cells including neutrophils, eosinophils, mast cells, dendritic cells, macrophages, and T and B-lymphocytes (Naert, R. et al., 2021, Int. J. Mol. Sci. 22, 11460, 1-17). TRPA1 expression is most prevalent in small diameter sensory neurons and it colocalizes with markers of peptidergic nociceptors such as TRPV1, calcitonin gene-related peptide (CGRP) and substance P (Kaneko, Y. et al., 2013, Curr. Top. Med. Chem. 13, 3, 241-243). TRPA1 functions primarily as a sensor for environmental irritants and is thought to give rise to somatosensory modalities such as pain, cold, and itch.

TRPA1 is activated by a range of endogenous and exogenous stimuli for pain and inflammation. Specifically, TRPA1 can be activated by external irritants such as allyl isothiocyanate (AITC) and allicin. TRPA1 can also be activated by cinnamaldehyde, which functions as an agonist to activate the channel through covalent modification of the cysteine residues in the N-terminal ankyrin repeats (Terrett, J. A. et al., 2021, J. Med. Chem. 64, 7, 3843-3869). TRPA1 can also be activated by noxious stimuli, including cold temperatures and pungent natural compounds such as mustard, cinnamon and garlic.

TRPA1 knock-out (KO) mouse models have implicated the ion channel in pain signaling. TRPA1 activity plays a role in a number of ailments in patients. A gain-of-function TRPA1 mutation in humans has been linked to familial episodic pain syndrome (FEPS) (Kremeyer, B. et al., 2010, Neuron 66, 5, 671-680). The discovery of a human genetic link between TRPA1 and FEPS suggests that TRPA1 plays a significant role in human pain. Patients carrying a single gain-of-function mutation in TRPA1 are known to experience debilitating upper body pain, triggered by fasting, cold, and fatigue. Several anesthetics are known to be TRPA1 agonists, including isoflurane (Matta, J. A. et al., 2008, PNAS 105, 25, 8784-8789) providing rationale for TRPA1 inhibitors for the relief of post-surgical pain.

TRPA1 activation has been implicated in the development of chronic respiratory diseases, including asthma and cough (Caceres, A. I. et al., 2009, Proc. Natl. Acad. Sci. 106, 22, 9099-104; Reese, R. M. et al., 2020, Scientific Reports 10, 979, 1-11). Airway hyperresponsiveness, bronchoconstriction and airway inflammation in asthma appear to be triggered by activity of TRPA1 expressed in airway smooth muscle cells, and the sensory nervous system and clinical symptoms can be relieved by TRPA1 antagonists (Balestrini, A. et al., 2021, J. Exp. Med. 218, 4, e20201637, 1-23; van den Berg, M. P. M. et al., 2021, Respir. Res. 22, 48, 1-15; Terrett, J. A. et al., 2021, J. Med. Chem. 64, 7, 3843-3869). The cough can be associated with asthma, chronic pulmonary obstructive disease (COPD), and idiopathic pulmonary fibrosis (IPF). The cough can also be post-viral cough or chronic idiopathic cough as well as cough in sensitive patients (Song, W.-J. and Chang, Y.-S., 2015, Clin. Transl. Allergy 5, 24, 1-10; Grace, M. S. and Belvisi, M. G., 2011, Pulm. Pharmacol. Ther. 24, 3, 286-288), however, TRPA-protective effects in IPF have also been reported (Virk, H. S. et al., 2021, Br J Pharmacol. 178, 2948-2962). TRPA1 antagonists can inhibit calcium signaling triggered by cough triggers such as cigarette smoke extract (CSE) oxidative stress, inflammatory mediator release and downregulated antioxidant gene expression (Lin, Y.-J. et al., 2015, J. Appl. Physiol. 118, 273-281; Wang, Z. et al., 2019, Front. Pharmacol. 10, 1253, 1-11).

TRPA1 has been implicated in dermatitis and itch. TRPA1 antagonists are effective in atopic dermatitis (Wilson, S. R. et al., 2013, J. Neurosci. 33, 22, 9283-9294), contact dermatitis (Liu, B. et al., 2013, FASEB J. 27, 9, 3549-3563), psoriasis-associated itch (Wilson, S. R. et al., 2013 J. Neurosci. 33, 22, 9283-9294), and IL-31-dependent itch (Cevikbas, F. et al., 2014, J. Allergy Clin. Immunol. 133, 2, 448-460). Direct clinical support for relief of AITC-induced itch upon TRPA1 specific inhibition has also been reported (Balestrini, A. et al., 2021, J. Exp. Med. 218, 4, e20201637, 1-23). Additionally, a TRPA1 antagonist is effective in a behavioral model of migraine-related allodynia (Edelmayer, R. M. et al., 2012, Pain 2012, 153, 9, 1949-1958).

TRPA1 expression is increased by inflammatory mediators and following nerve injury suggesting a role for TRPA1 activity in inflammation. For example, TRPA1 is required for the observed hypersensitivity in inflammatory pain models (Bautista, D. M. et al. 2013, Annu. Rev. Physiol. 75, 181-200; Julius, D. 2013, Annu. Rev. Cell Dev. Biol. 29, 355-384). Disease models of diabetes indicate that TRPA1 plays a role in the inflammatory pain associated with this metabolic disorder. TRPA1 may also have a role in the pathogenesis of cancer and other inflammatory diseases. Studies further suggest that TRPA1 is implicated in migraine pain as a result from neurogenic inflammation (Edelmayer, R. M. et al., 2012, Pain 153, 9, 1949-1958). This may be due to the activation of trigeminal TG neurons through nasal application of TRPA1 activators.

TRPA1 also plays a role in arthritis and osteoarthritic pain (Horvath, A. et al., 2016, Arthritis Res. Ther. 18, 6, 1-14). Activation of TRPA1 has been shown to elicit an inflammatory response in osteoarthritic chondrocytes (Nummenmaa, E. et al., 2016, Arthritis Res. Ther. 18, 185). This is supported by observations that TRPA1 inhibition and genetic deletion reduces knee swelling, histopathological destruction, and inflammatory mediators in osteoarthritic mouse chondrocytes and murine cartilage (Nummenmaa, E. et al., 2016, Arthritis Res. Ther. 18, 185, 1-11; Horvath, A. et al., 2016, Arthritis Res. Ther. 18, 6, 1-14). Additionally, TRPA1 KO mice have been shown to improve in weight bearing on the osteoarthritic limb in a knee swelling model (Horvath, A. et al., 2016, Arthritis Res. Ther. 18, 6).

TRPA1 also has a role in colitis and visceral hypersensitivity and in mediating gastrointestinal (GI) hypersensitivity to mechanical stimuli. TRPA1 expression is elevated in the inflamed mouse gut (Cseko, K. et al., 2019, Pharmaceuticals 12, 48, 1-19; Izzo, A. et al., 2012, Br. J. Pharmacol. 166, 4, 1444-1460). Additionally, colitis induced by dinitrobenzene sulphonic acid (DNBS) is attenuated after pharmacological blockade or genetic inactivation of TRPA1 (Engel, M. A. et al., 2011, Gastroenterology 141, 4, 1346-1358), suggesting that TRPA1 can be a target in Gl inflammatory conditions such as inflammatory bowel disease, Crohn's disease and ulcerative colitis (Cseko, K. et al., 2019, Pharmaceuticals 12, 48, 1-19; Blackshaw, L. A. et al., 2013, The Open Pain Journal 6, (Suppl 1: M4) 23-30).

TRPA1 is highly expressed in sensory neurons innervating the bladder, suggesting that TRPA1 is a potential drug target for bladder disorders such as bladder instability, urinary incontinence, and cystitis (Streng, T. et al., 2008, Eur. Urol. 53, 391-399). TRPA1 is up-regulated in bladder mucosa in patients with bladder outlet obstruction (Du, S. et al., 2008, Urology 72, 2, 450-455).

Thus, there remains a need for development of novel TRPA1 inhibitors as pharmaceutical agents for the treatment of a number of conditions, disorders, and diseases.

### SUMMARY OF THE INVENTION

In one aspect, compounds useful as TRPA1 inhibitors having a structure of Formula I

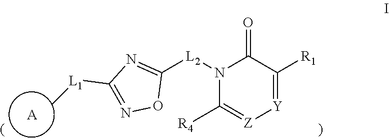

are described, where the various substituents are defined herein. The compounds of Formula I described herein can block inhibit TRPA1 and be used in the treatment of a variety of conditions. Methods for synthesizing these compounds are also described herein. Pharmaceutical compositions and methods of using these compositions described herein are useful for treating conditions in vitro and in vivo. Such compounds, pharmaceutical compositions, and methods of treatment have a number of clinical applications, including as pharmaceutically active agents and methods for treating pain, a skin disorder, a respiratory disease, a fibrotic disease, an inner ear disorder, fever or another disorder of thermoregulation, a urinary tract disorder, an autoimmune disease, ischemia, a central nervous system (CNS) disorder, an inflammatory disorder, a gastroenterological disorder, and a cardiovascular disorder, or a combination thereof.

In one aspect, a compound of Formula I or a pharmaceutically acceptable salt thereof, or a tautomer is described,

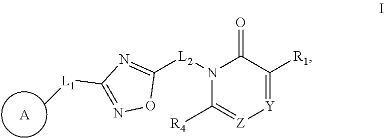

wherein
Y is N or CR₂;
Z is N or CR₃;
R₁ is H, D, halogen, alkyl, cycloalkyl, halogenated alkyl, halogenated cycloalkyl, saturated heterocycle, CN, ORₐ, SRₐ, or NRₐR_{b};
R₂ is H, D, halogen, alkyl, alkenyl, alkynyl, cycloalkyl, halogenated alkyl, halogenated alkenyl, halogenated alkynyl, halogenated cycloalkyl, saturated heterocycle, partially saturated heterocycle, aryl, heteroaryl, alkylaryl, alkylheteroaryl, CN, —C₁₋₄alkyl-CN, ORₐ, SRₐ, NRₐR_{b}, (C═O)NRₐR_{b}, NR_{b}(C═O)Rₐ, (C═O)Rₐ, (C═O)ORₐ, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b};
R₃ is H, D, halogen, alkyl, alkenyl, alkynyl, cycloalkyl, halogenated alkyl, halogenated alkenyl, halogenated alkynyl, halogenated cycloalkyl, saturated heterocycle, partially saturated heterocycle, aryl, heteroaryl, alkylaryl, alkylheteroaryl, CN, —C₁₋₄alkyl-CN, ORₐ, SRₐ, NRₐR_{b}, (C═O)NRₐR_{b}, NR_{b}(C═O)Rₐ, (C═O)Rₐ, (C═O)ORₐ, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b};
R₄ is H, D, halogen, alkyl, cycloalkyl, halogenated alkyl, halogenated cycloalkyl, aryl, heteroaryl, saturated heterocycle, CN, ORₐ, SRₐ, —C₁₋₄alkyl-ORₐ, or NRₐR_{b};
\textcircled{A} is an aryl or heteroaryl each optionally substituted by 1-5 substituents each independently selected from the group consisting of H, D, halogen, alkyl, cycloalkyl, halogenated cycloalkyl, halogenated alkyl, alkenyl, alkynyl, aryl, heteroaryl, CN, ORₐ, SRₐ, NRₐR_{b}, —C₁₋₄alkyl-SRₐ, or —C₁₋₄alkyl-ORₐ;
L₁ is —(CR₅R₆)ₙ—;
   each occurrence of R₅ is independently H, D, alkyl, halogen, halogenated alkyl, cycloalkyl, halogenated cycloalkyl, CN, ORₐ, or —C₁₋₄alkyl-ORₐ;
   each occurrence of R₆ is independently H, D, alkyl, halogen, halogenated alkyl, cycloalkyl, halogenated cycloalkyl, CN, ORₐ, or —C₁₋₄alkyl-ORₐ;
   n is 2 or 3;
L₂ is —CR₇R₈—;
   R₇ is H, D, alkyl, halogenated alkyl, cycloalkyl, halogenated cycloalkyl, CN, or —C₁₋₄alkyl-ORₐ;
   R₈ is H, D, alkyl, halogenated alkyl, cycloalkyl, halogenated cycloalkyl, CN, or —C₁₋₄alkyl-ORₐ;
each occurrence of Rₐ and R_{b} are independently H, alkyl, (C═O)Rₓ, (C═O)N(Rₓ)₂, SO₂Rₓ, NRₓ(C═O)NRₓ₂, cycloalkyl, halogenated alkyl, heteroalkyl, halogenated heteroalkyl, halogenated cycloalkyl, saturated heterocycle comprising 1-3 heteroatoms each selected from the group consisting of N, O, and S, aryl, or heteroaryl; or alternatively Rₐ and R_{b} together with the carbon or nitrogen atom that they are connected to form a cycloalkyl or saturated heterocycle comprising the nitrogen atom and 0-3 additional heteroatoms each selected from the group consisting of N, O, and S;
the alkyl, alkenyl, alkynyl, cycloalkyl, saturated heterocycle, partially saturated heterocycle, aryl, heteroaryl, alkylaryl, and alkylheteroaryl in R₁, R₂, R₃, R₄, R₅, R₆, R₇, R₈, Rₐ, or R_{b}, where applicable, are optionally substituted by 1-4 substituents each independently selected from the group consisting of alkyl, cycloalkyl, halogenated cycloalkyl, halogenated alkyl, halogen, CN, ORₓ, —(CH₂)₁₋₂ORₓ, N(Rₓ)₂, —(CH₂)₁₋₂N(Rₓ)₂, (C═O)Rₓ, (C═O)N(Rₓ)₂, NRₓ(C═O)Rₓ, and oxo where valence permits; and
each occurrence of Rₓ is independently H, D, alkyl, or optionally substituted heterocycle; or alternatively the two Rₓ groups together with the nitrogen atom that they are connected to form a heterocycle optionally substituted by alkyl and comprising the nitrogen atom and 0-3 additional heteroatoms each selected from the group consisting of N, O, and S;
provided that when Z is N, R₄ is not OH.

In any one of the embodiments described herein, Y is CR₂.

In any one of the embodiments described herein, Y is N.

In any one of the embodiments described herein, Z is CR₃.

In any one of the embodiments described herein, Z is N.

In any one of the embodiments described herein, n is 2.

In any one of the embodiments described herein, each occurrence of R₅ is independently cycloalkyl, halogenated cycloalkyl, —C₁₋₄alkyl-ORₐ, or CN.

In any one of the embodiments described herein, each occurrence of R₅ is independently H, D, alkyl, halogen, ORₐ, or fluorinated alkyl.

In any one of the embodiments described herein, each occurrence of R₅ independently H, D, CH₃, CH₂CH₃, OH, F, Cl, or Br.

In any one of the embodiments described herein, each occurrence of R₆ is independently cycloalkyl, halogenated cycloalkyl, —C₁₋₄alkyl-ORₐ, or CN.

In any one of the embodiments described herein, each occurrence of R₆ is independently H, D, alkyl, halogen, ORₐ, or fluorinated alkyl.

In any one of the embodiments described herein, each occurrence of R₆ independently H, D, CH₃, CH₂CH₃, OH, F, Cl, or Br.

In any one of the embodiments described herein, L₁ is selected from the group consisting of —CH₂—CH₂—, —CH(CH₃)—CH₂—, —CH₂—CH(CH₃)—, —CH₂—C(CH₃)₂—, —CH(OH)—CH₂—, —CH₂—CH(OH)—,

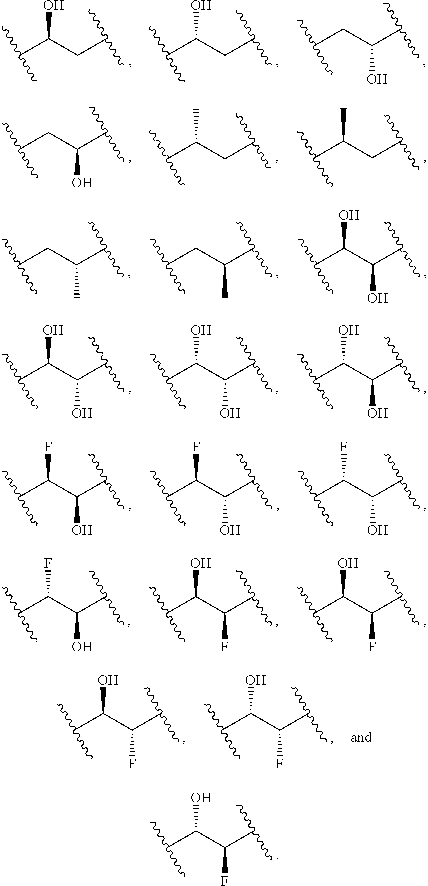

In any one of the embodiments described herein, L₁ is selected from the group consisting of —CH₂—CH₂—,

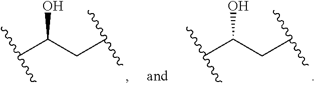 [SMILES: *C[C@@H](*)O ; *C[C@H](*)O]

In any one of the embodiments described herein, the compound has the structure of Formula Ia, Ib, or Ic:

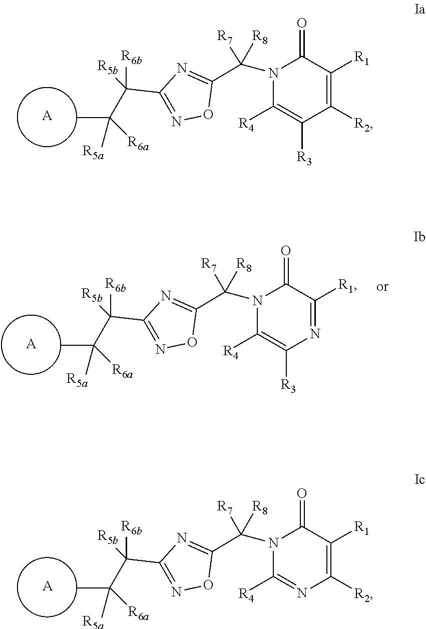

wherein
each occurrence of R₅ₐ is independently H, D, alkyl, halogen, ORₐ, or fluorinated alkyl;
each occurrence of R_{5b} is independently H, D, alkyl, halogen, ORₐ, or fluorinated alkyl;
each occurrence of R₆ₐ is independently H, D, alkyl, halogen, ORₐ, or fluorinated alkyl; and
each occurrence of R_{6b} is independently H, D, alkyl, halogen, ORₐ, or fluorinated alkyl.

In any one of the embodiments described herein, R₇ is cycloalkyl, halogenated cycloalkyl, CN, or —C₁₋₄alkyl-ORₐ.

In any one of the embodiments described herein, R₇ is H, D, alkyl, or fluorinated alkyl.

In any one of the embodiments described herein, R₇ is H, D, CH₃, or CH₂CH₃.

In any one of the embodiments described herein, R₈ is cycloalkyl, halogenated cycloalkyl, CN, or —C₁₋₄alkyl-ORₐ.

In any one of the embodiments described herein, R₈ is H, D, alkyl, or fluorinated alkyl.

In any one of the embodiments described herein, R₈ is H, CH₃, or CH₂CH₃.

In any one of the embodiments described herein, the structural moiety L₂ is selected from the group consisting of —CH₂—, —CH(CH₃)—, —C(CH₃)₂—, and —CH(CH₂CH₃).

In any one of the embodiments described herein, L₂ is —CH₂—.

In any one of the embodiments described herein, is phenyl which is optionally substituted with by 1-5 substituents each independently selected from the group consisting of H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, and —C₁₋₄alkyl-ORₐ.

In any one of the embodiments described herein, the compound has the structure of Formula IIa, IIb, or IIc:

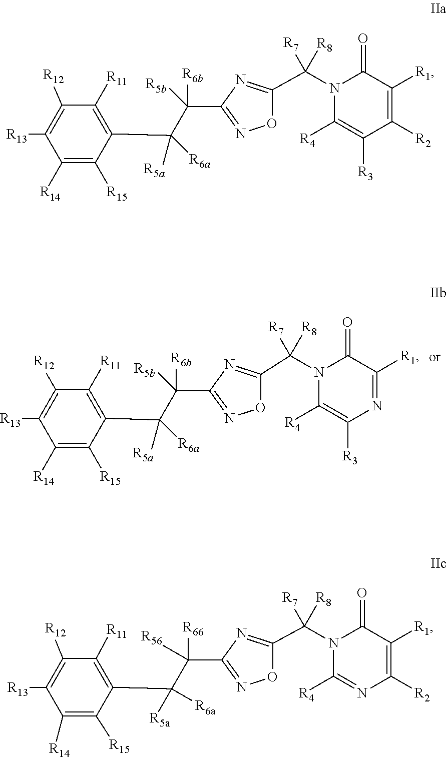

wherein
each occurrence of R₅ₐ is independently H, D, alkyl, halogen, ORₐ, or fluorinated alkyl;
each occurrence of R_{5b} is independently H, D, alkyl, halogen, ORₐ, or fluorinated alkyl;
each occurrence of R₆ₐ is independently H, D, alkyl, halogen, ORₐ, or fluorinated alkyl;
each occurrence of R_{6b} is independently H, D, alkyl, halogen, ORₐ, or fluorinated alkyl;
each occurrence of Ru is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ;
each occurrence of R₁₂ is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ;
each occurrence of R₁₃ is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ;
each occurrence of R₁₄ is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ; and
each occurrence of R₁₅ is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ.

In any one of the embodiments described herein, R₁₁, R₁₂, R₁₄, and R₁₅ are H; and R₁₃ is H, D, halogen, alkyl, alkenyl, alkynyl, cycloalkyl, CN, CF₃, ORₐ, SRₐ, NRₐR_{b}, or —C₁₋₄alkyl-ORₐ.

In any one of the embodiments described herein, R₁₃ is CH₃, CH₂CH₃, OH, F, Cl, Br, OCH₃, CH₂OCH₃, CF₃, CN, C≡CH, or

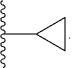 [SMILES: *C1CC1]

In any one of the embodiments described herein, \textcircled{A} is selected from the group consisting of

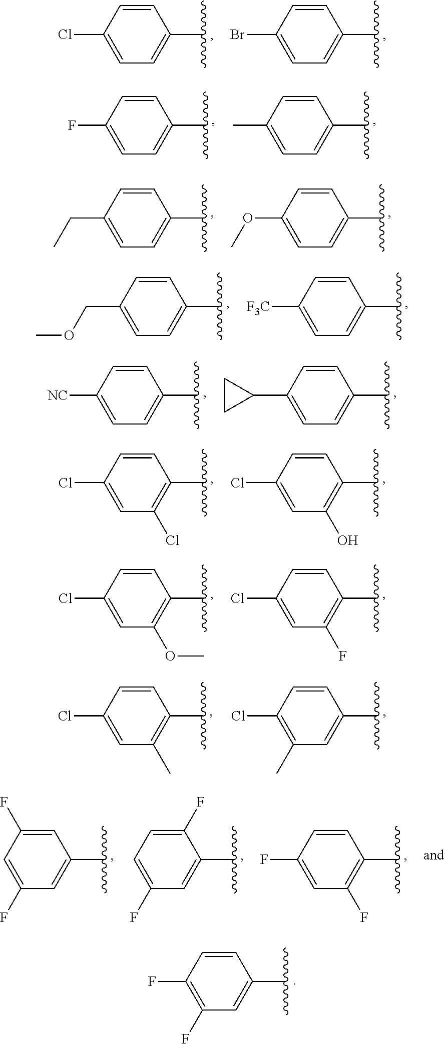

In any one of the embodiments described herein, \textcircled{A} is a 5- or 6-membered heteroaryl which is optionally substituted with by 1-4 substituents each independently selected from the group consisting of H, halogen, alkyl, cycloalkyl, halogenated cycloalkyl, halogenated alkyl, aryl, heteroaryl, CN, ORₐ, SRₐ, NRₐR_{b}, and —C₁₋₄alkyl-ORₐ.

In any one of the embodiments described herein, is selected from the group consisting of

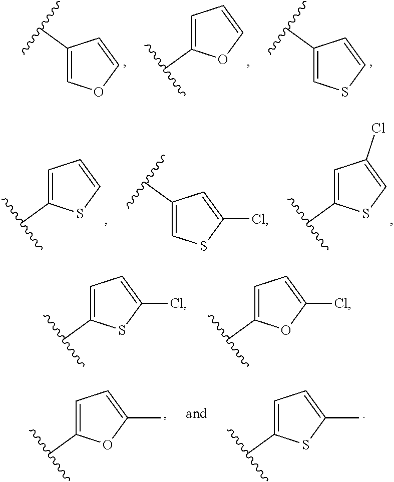

In any one of the embodiments described herein, R₁ is cycloalkyl, halogenated alkyl, or halogenated cycloalkyl.

In any one of the embodiments described herein, R₁ is H, D, halogen, alkyl, CN, CF₃, ORₐ, SRₐ, or NRₐR_{b}.

In any one of the embodiments described herein, R₁ is selected from the group consisting of H, D, CH₃, CH₂CH₃, OH, F, Cl, Br, OCH₃, CF₃, CN, NH₂, NHCH₃, N(CH₃)₂, and

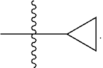 [SMILES: *C1CC1]

In any one of the embodiments described herein, R₂ is H, D, halogen, CN, CF₃, ORₐ, SRₐ, NRₐR_{b}, (C═O)NRₐR_{b}, NR_{b}(C═O)Rₐ, (C═O)Rₐ, (C═O)ORₐ, —C₁₋₄alkyl-CN, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, 0-C₁₋₄alkyl-Rₐ, or NRₐ—C₁₋₄alkyl-R_{b}.

In any one of the embodiments described herein, R₂ is saturated heterocycle, partially saturated heterocycle, or heteroaryl, each optionally substituted with 1-3 substituents selected from the group consisting of halogen, alkyl, CN, ORₓ, —(CH₂)₁₋₂ORₓ, N(Rₓ)₂, —(CH₂)₁₋₂N(Rₓ)₂, (C═O)Rₓ, (C═O)N(Rₓ)₂, NRₓ(C═O)Rₓ, and oxo where valence permits.

In any one of the embodiments described herein, R₂ is alkyl, alkenyl, or alkynyl, each optionally substituted with 1-3 substituents selected from the group consisting of halogen, CN, ORₓ, —(CH₂)₁₋₂ORₓ, N(Rₓ)₂, —(CH₂)₁₋₂N(Rₓ)₂, (C═O)Rₓ, (C═O)N(Rₓ)₂, NRₓ(C═O)Rₓ, and oxo where valence permits.

In any one of the embodiments described herein, R₂ is cycloalkyl, aryl, or alkylaryl, alkylheteroaryl.

In any one of the embodiments described herein, R₂ is selected from the group consisting of H, D, CH₃, CH₂CH₃, OH, F, Cl, Br, I, OCH₃, CF₃, CN, NH₂, NHCH₃, N(CH₃)₂, CH═CH₂,

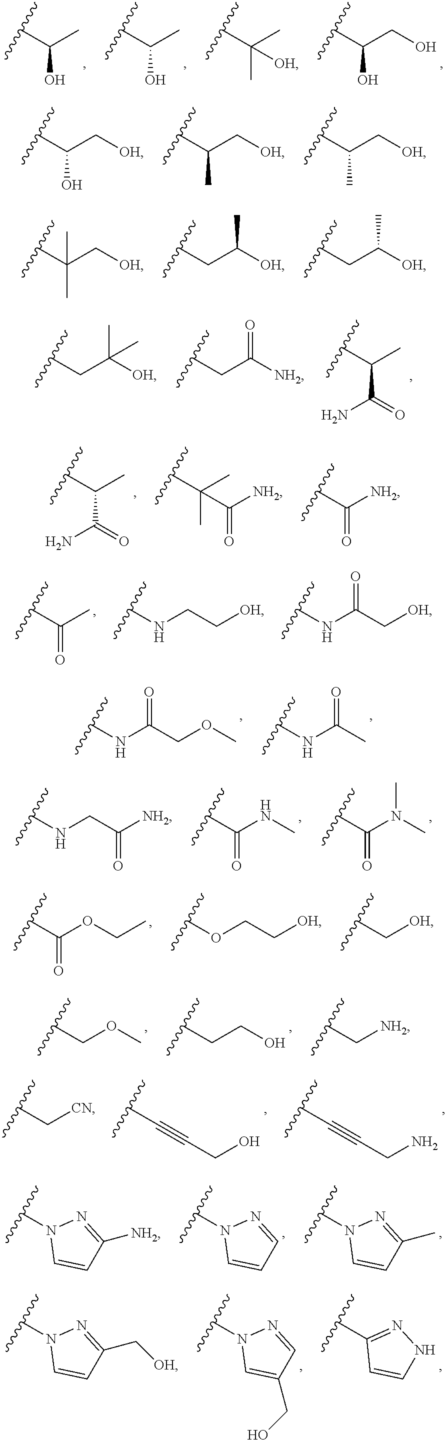

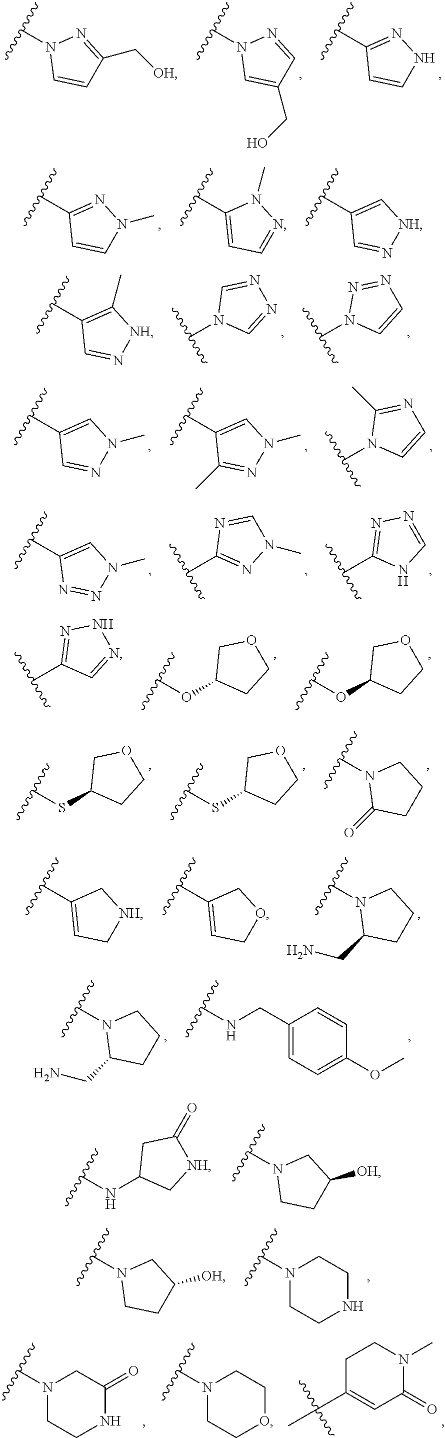

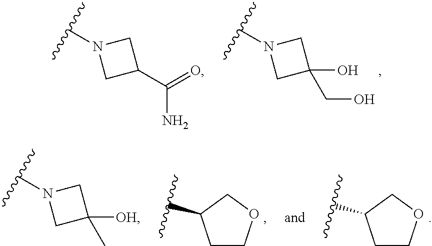

In any one of the embodiments described herein, R₃ is H, D, halogen, alkyl, halogenated alkyl, heteroaryl, or CN.

In any one of the embodiments described herein, R₃ is ORₐ, SRₐ, NRₐR_{b}, (C═O)NRₐR_{b}, —C₁₋₄alkyl-CN, —C₁₋₄alkyl-ORₐ, —C₁₋₄alkyl-SRₐ, —C₁₋₄alkyl-NRₐR_{b}, or —C₁₋₄alkyl-CONRₐR_{b}.

In any one of the embodiments described herein, R₃ is alkenyl, alkynyl, cycloalkyl, saturated heterocycle, partially saturated heterocycle, aryl, alkylaryl, alkylheteroaryl, NR_{b}(C═O)Rₐ, (C═O)Rₐ, (C═O)ORₐ, —C₁₋₄alkyl-COORₐ, —C₁₋₄alkyl-NRₐCOR_{b}, O—C₁₋₄alkyl-Rₐ, or NRₐ—C₁₋₄alkyl-R_{b}.

In any one of the embodiments described herein, R₃ is selected from the group consisting of H, D, CH₃, CH₂CH₃, OH, F, Cl, Br, OCH₃, CF₃, CN, CH₂CN, CH═CH₂, NH₂, NHCH₃, N(CH₃)₂,

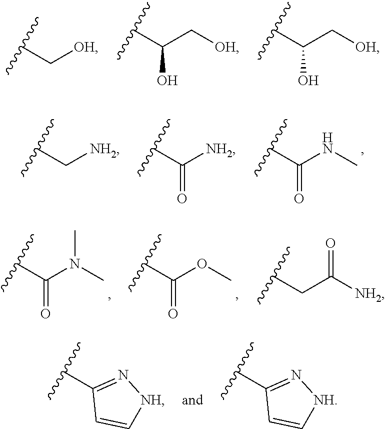

In any one of the embodiments described herein, R₄ is cycloalkyl, halogenated alkyl, or halogenated cycloalkyl.

In any one of the embodiments described herein, R₄ is H, D, halogen, alkyl, CN, CF₃, ORₐ, SRₐ, —C₁₋₄alkyl-ORₐ, or NRₐR_{b}.

In any one of the embodiments described herein, R₄ is selected from the group consisting of H, D, CH₃, CH₂CH₃, OH, F, Cl, Br, OCH₃, CF₃, CN, NH₂, NHCH₃, N(CH₃)₂, CH₂OH,

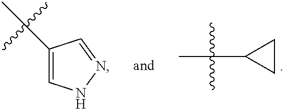

In any one of the embodiments described herein, at least one occurrence of Rₐ or R_{b} is independently H, alkyl, cycloalkyl, saturated heterocycle, aryl, or heteroaryl.

In any one of the embodiments described herein, at least one occurrence of Rₐ or R_{b} is independently H, D, Me, Et, Pr, CH₂CH₂OH, phenyl, or a heterocycle selected from the group consisting of

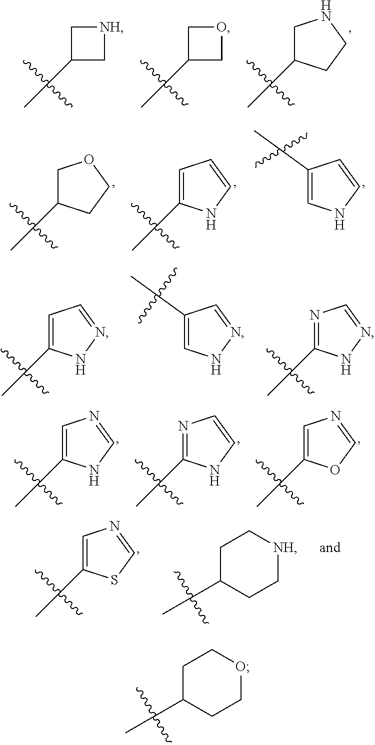

wherein the heterocycle is optionally substituted by alkyl, OH, oxo, or (C═O)C₁₋₄alkyl where valence permits.

In any one of the embodiments described herein, at least one occurrence of Rₐ or R_{b} is H, Me, phenyl,

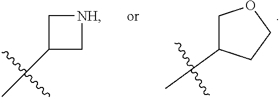 [SMILES: *C1CNC1 ; *C1CCOC1]

In any one of the embodiments described herein, Rₐ and R_{b} together with the nitrogen atom that they are connected to form an optionally substituted heterocycle comprising the nitrogen atom and 0-3 additional heteroatoms each selected from the group consisting of N, O, and S.

In any one of the embodiments described herein, each occurrence of Rₓ is independently H, alkyl, or heterocycle optionally substituted by alkyl, halogen, or OH.

In any one of the embodiments described herein, Rₓ is independently H or alkyl.

In any one of the embodiments described herein, Rₓ is independently H or Me.

In any one of the embodiments described herein, the compound is selected from the group consisting of compounds in Examples 2-5 and Tables 1-5.

In another aspect, a pharmaceutical composition is described, including at least one compound according to any one of the embodiments described herein or a pharmaceutically acceptable salt thereof and a pharmaceutically acceptable carrier or diluent.

In yet another aspect, a method of treating a condition in a mammalian species in need thereof is described, including administering to the mammalian species a therapeutically effective amount of at least one compound according to any one of the embodiments described herein, or a pharmaceutically acceptable salt thereof, or a pharmaceutical composition thereof, where the condition is selected from the group consisting of pain, a skin disorder, a respiratory disease, a fibrotic disease, an inner ear disorder, fever or another disorder of thermoregulation, a urinary tract or bladder disorder, an autoimmune disease, ischemia, a central nervous system (CNS) disorder, an inflammatory disorder, a gastroenterological disorder, and a cardiovascular disorder.

In any one of the embodiments described herein, the pain is acute pain, chronic pain, complex regional pain syndrome, inflammatory pain, neuropathic pain, postoperative pain, rheumatoid arthritic pain, osteoarthritis pain, back pain, visceral pain, cancer pain, algesia, neuralgia, migraine, neuropathies, diabetic neuropathy, sciatica, HIV-related neuropathy, pos-herpetic neuralgia, fibromyalgia, nerve injury, post stock pain, or tooth and tooth injury-related pain.

In any one of the embodiments described herein, the urinary tract or bladder disorder is pelvic hypersensitivity, urinary incontinence, cystitis, bladder instability, or bladder outlet obstruction.

In any one of the embodiments described herein, the skin disorder is burns, psoriasis, eczema, or pruritus.

In any one of the embodiments described herein, the skin disorder is atopic dermatitis or psoriasis-induced itching.

In any one of the embodiments described herein, the respiratory disease is an inflammatory airway disease, airway hyperresponsiveness, an idiopathic lung disease, chronic obstructive pulmonary disease, asthma, chronic asthma, tracheobronchial or diaphragmatic dysfunction, cough, or chronic cough.

In any one of the embodiments described herein, the ischemia is CNS hypoxia or a disorder associated with reduced blood flow to CNS.

In any one of the embodiments described herein, the autoimmune disease is rheumatoid arthritis or multiple sclerosis.

In any one of the embodiments described herein, the central nervous system disorder is associated with neurodegeneration.

In any one of the embodiments described herein, the gastroenterological disorder is an inflammatory bowel disease, esophagitis, gastroesophageal reflux disorder, irritable bowel syndrome, emesis, or stomach duodenal ulcer.

In any one of the embodiments described herein, the cardiovascular disorder is stroke, myocardial infarction, atherosclerosis, or cardiac hypertrophy.

In any one of the embodiments described herein, the mammalian species is human.

In yet another aspect, a method of inhibiting transient receptor potential ankyrin 1 (TRPA1) in a mammalian species in need thereof is described, including administering to the mammalian species a therapeutically effective amount of at least one compound according to any one of the embodiments described herein, or a pharmaceutically acceptable salt thereof, or a pharmaceutical composition thereof.

In any one of the embodiments described herein, the mammalian species is human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art. It is to be understood that the terminology used herein is for the purpose of describing certain embodiments only and is not intended to be limiting.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-Cₓ)alkyl” or “C₁₋ₓalkyl” refers to a straight or branched chain alkane (hydrocarbon) radical containing from 1 to x carbon atoms. For example, the term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle, and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle, and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-Cₓ alkenyl” or “C₂₋ₓalkenyl” refers to a straight or branched chain hydrocarbon radical containing from 2 to x carbon atoms and at least one carbon-carbon double bond. For example, the term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited to, one or more of the following groups: hydrogen, halogen, alkyl, halogenated alkyl (i.e., an alkyl group bearing a single halogen substituent or multiple halogen substituents such as CF₃ or CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary groups include ethynyl. The term “C₂-Cₓalkynyl” or “C₂₋ₓ alkynyl” refers to a straight or branched chain hydrocarbon radical containing from 2 to x carbon atoms and at least one carbon-carbon triple bond. For example, the term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” or “C₃₋₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}Rₑ, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}Rₑ, OC(═O)Rₐ, OC(═O)NR_{b}Rₑ, NR_{b}C(═O)ORₑ, NR_{d}C(═O)NR_{b}Rₑ, NR_{d}S(═O)₂NR_{b}Rₑ, NR_{d}P(═O)₂NR_{b}Rₑ, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}Rₑ, NR_{b}S(═O)₂Rₑ, NR_{b}P(═O)₂Rₑ, S(═O)₂NR_{b}Rₑ, P(═O)₂NR_{b}Rₑ, C(═O)OR_{d}, C(═O)Rₐ, C(═O)NR_{b}Rₑ, OC(═O)Rₐ, OC(═O)NR_{b}Rₑ, NR_{b}C(═O)ORₑ, NR_{d}C(═O)NR_{b}Rₑ, NR_{d}S(═O)₂NR_{b}Rₑ, NR_{d}P(═O)₂NR_{b}Rₑ, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings wherein two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}Rₑ, OC(═O)Rₐ, OC(═O)NR_{b}Rₑ, NR_{b}C(═O)ORₑ, NRₐC(═O)NR_{b}Rₑ, NR_{d}S(═O)₂NR_{b}Rₑ, NRₐP(═O)₂NR_{b}Rₑ, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms and sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, indolinyl, isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “oxo” refers to

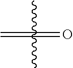 [SMILES: *=O]

substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be rearranged to satisfy the valence requirement. For instance, a pyridine with a 2-oxo substituent group may have the structure of

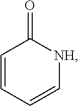

which also includes its tautomeric form of

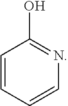

The term “alkylamino” refers to a group having the structure —NHR′, wherein R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,2,4-triazolyl, and tetrazolyl.

The terms “halogen” or “halo” refer to chlorine, bromine, fluorine, or iodine.

The term “substituted” refers to the embodiments in which a molecule, molecular moiety, or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a phenol or carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates, or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid; for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a phenol or carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides, and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention. As used herein, any depicted structure of the compound includes the tautomeric forms thereof.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to or greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999), the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H (T), and carbon-14, i.e., ¹⁴C, isotopes are particularly preferred for their ease of preparation and detectability. Further, substitution with heavier isotopes such as deuterium, i.e., ²H (D), can afford certain therapeutic advantages resulting from greater metabolic stability, for example, increased in vivo half-life or reduced dosage requirements, and hence may be preferred in some circumstances. Isotopically labeled compounds can generally be prepared by carrying out the procedures disclosed in the Schemes and/or in the Examples below, by substituting a readily available isotopically labeled reagent for a non-isotopically labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; cardio-facio-cutaneous syndrome; hereditary gingival fibromatosis type 1; autoimmune lymphoproliferative syndrome; and capillary malformation-arterovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

### Compounds

Novel compounds as TRPA1 inhibitors are described. It has been surprisingly discovered that the compounds disclosed herein exhibit TRPA1 inhibiting properties. Additionally, it has been surprisingly discovered that the compounds disclosed herein selectively block TRPA1 and do not block the hERG channel and thus have desirable cardiovascular safety profiles.

In one aspect, a compound having a structure of Formula I, Ia, Ib, Ic, IIa, IIb, or IIc

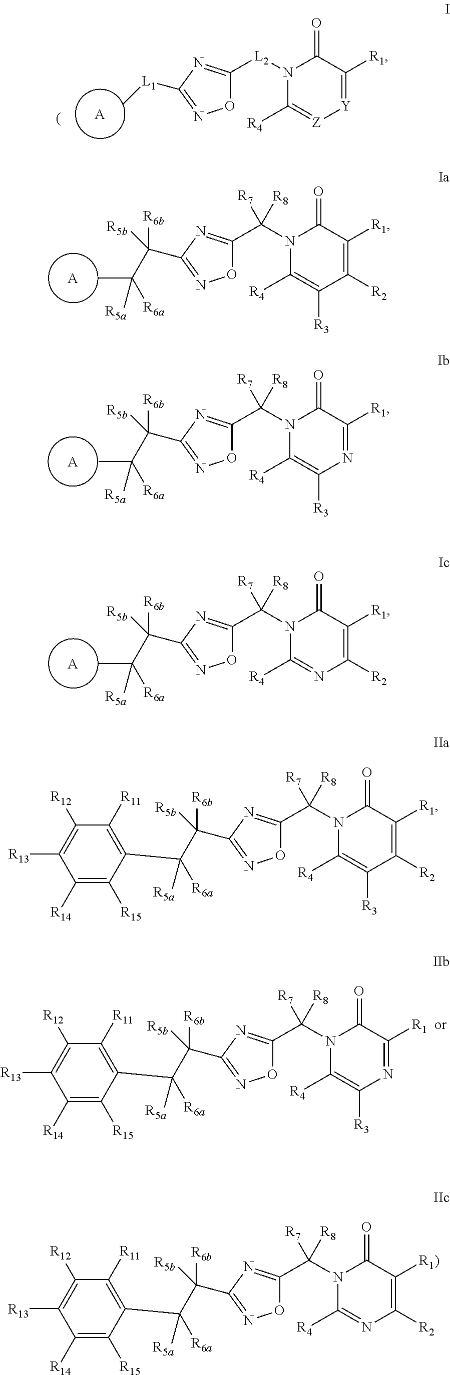

or a pharmaceutically acceptable salt thereof, or a tautomer thereof is described, where the various substituents are defined herein. The compounds of Formula I, Ia, Ib, Ic, IIa, IIb, or IIc described herein can block or inhibit TRPA1 and be used in the treatment of a variety of conditions. Methods for synthesizing these compounds are also described herein. Pharmaceutical compositions including the compound described herein and methods of using these compositions described herein are useful for treating conditions in vitro and in vivo. Such compounds, pharmaceutical compositions, and methods of treatment have a number of clinical applications, including as pharmaceutically active agents and methods for treating pain, a skin disorder, a respiratory disease, a fibrotic disease, an inner ear disorder, fever or another disorder of thermoregulation, a urinary tract disorder, an autoimmune disease, ischemia, a central nervous system (CNS) disorder, an inflammatory disorder, a gastroenterological disorder, and a cardiovascular disorder, or a combination thereof.

In one aspect, a compound of Formula I or a pharmaceutically acceptable salt, or a tautomer thereof is described,

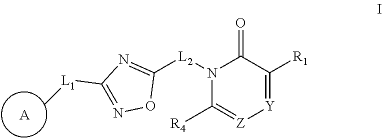

wherein
Y is N or CR₂;
Z is N or CR₃;
R₁ is H, D, halogen, alkyl, cycloalkyl, halogenated alkyl, halogenated cycloalkyl, saturated heterocycle, CN, ORₐ, SRₐ, or NRₐR_{b};
R₂ is H, D, halogen, alkyl, alkenyl, alkynyl, cycloalkyl, halogenated alkyl, halogenated alkenyl, halogenated alkynyl, halogenated cycloalkyl, saturated heterocycle, partially saturated heterocycle, aryl, heteroaryl, alkylaryl, alkylheteroaryl, CN, —C₁₋₄alkyl-CN, ORₐ, SRₐ, NRₐR_{b}, (C═O)NRₐR_{b}, NR_{b}(C═O)Rₐ, (C═O)Rₐ, (C═O)ORₐ, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b};
R₃ is H, D, halogen, alkyl, alkenyl, alkynyl, cycloalkyl, halogenated alkyl, halogenated alkenyl, halogenated alkynyl, halogenated cycloalkyl, saturated heterocycle, partially saturated heterocycle, aryl, heteroaryl, alkylaryl, alkylheteroaryl, CN, —C₁₋₄alkyl-CN, ORₐ, SRₐ, NRₐR_{b}, (C═O)NRₐR_{b}, NR_{b}(C═O)Rₐ, (C═O)Rₐ, (C═O)ORₐ, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b};
R₄ is H, D, halogen, alkyl, cycloalkyl, aryl, heteroaryl, halogenated alkyl, halogenated cycloalkyl, saturated heterocycle, CN, ORₐ, SRₐ, —C₁₋₄alkyl-ORₐ, or NRₐR_{b};
\textcircled{A} is an aryl or heteroaryl each optionally substituted by 1-5 substituents each independently selected from the group consisting of H, D, halogen, alkyl, cycloalkyl, halogenated cycloalkyl, halogenated alkyl, alkenyl, alkynyl, aryl, heteroaryl, CN, ORₐ, SRₐ, NRₐR_{b}, —C₁₋₄alkyl-SRₐ, or —C₁₋₄alkyl-ORₐ;
L₁ is —(CR₅R₆)ₙ—;
   each occurrence of R₅ is independently H, D, alkyl, halogen, halogenated alkyl, cycloalkyl, halogenated cycloalkyl, CN, ORₐ, or —C₁₋₄alkyl-ORₐ;
   each occurrence of R₆ is independently H, D, alkyl, halogen, halogenated alkyl, cycloalkyl, halogenated cycloalkyl, CN, ORₐ, or —C₁₋₄alkyl-ORₐ;
   n is 2 or 3;
L₂ is —CR₇R₈—;
   R₇ is H, D, alkyl, halogenated alkyl, cycloalkyl, halogenated cycloalkyl, CN, or —C₁₋₄alkyl-ORₐ;
   R₈ is H, D, alkyl, halogenated alkyl, cycloalkyl, halogenated cycloalkyl, CN, or —C₁₋₄alkyl-ORₐ;
each occurrence of Rₐ and R_{b} are independently H, alkyl, (C═O)Rₓ, (C═O)N(Rₓ)₂, SO₂Rₓ, NRₓ(C═O)NRₓ₂, cycloalkyl, halogenated alkyl, heteroalkyl, halogenated heteroalkyl, halogenated cycloalkyl, saturated heterocycle comprising 1-3 heteroatoms each selected from the group consisting of N, O, and S, aryl, or heteroaryl; or alternatively Rₐ and R_{b} together with the carbon or nitrogen atom that they are connected to form a cycloalkyl or saturated heterocycle comprising the nitrogen atom and 0-3 additional heteroatoms each selected from the group consisting of N, O, and S;
the alkyl, alkenyl, alkynyl, cycloalkyl, saturated heterocycle, partially saturated heterocycle, aryl, heteroaryl, alkylaryl, and alkylheteroaryl in R₁, R₂, R₃, R₄, R₅, R₆, R₇, R₈, Rₐ, or R_{b}, where applicable, are optionally substituted by 1-4 substituents each independently selected from the group consisting of alkyl, cycloalkyl, halogenated cycloalkyl, halogenated alkyl, halogen, CN, ORₓ, —(CH₂)₁₋₂ORₓ, N(Rₓ)₂, —(CH₂)₁₋₂N(Rₓ)₂, (C═O)Rₓ, (C═O)N(Rₓ)₂, NRₓ(C═O)Rₓ, and oxo where valence permits; and
each occurrence of Rₓ is independently H, D, alkyl, or optionally substituted heterocycle; or alternatively the two Rₓ groups together with the nitrogen atom that they are connected to form a heterocycle optionally substituted by alkyl and comprising the nitrogen atom and 0-3 additional heteroatoms each selected from the group consisting of N, O, and S;
provided that when Z is N, R₄ is not OH.

In some embodiments, Y is CR₂. In some embodiments, Y is N. In some embodiments, Z is CR₃. In some embodiments, Z is N. In some embodiments, Y is CR₂ and Z is N. In some embodiments, Y is N and Z is CR₃. In some embodiments, Y is CR₂ and Z is CR₃. In some embodiments, Y is N and Z is N.

In some embodiments, n is 2. In some embodiments, n is 3.

In some embodiments, each occurrence of R₅ is independently H, D, alkyl, halogenated alkyl, cycloalkyl, halogenated cycloalkyl, CN, ORₐ, —C₁₋₄alkyl-ORₐ, or halogen. In some embodiments, each occurrence of R₅ is independently cycloalkyl, halogenated cycloalkyl, or CN. In some embodiments, each occurrence of R₅ is independently H, D, alkyl, halogen, ORₐ, or fluorinated alkyl. In some embodiments, at least one occurrence of R₅ is H or D. In some embodiments, at least one occurrence of R₅ is ORₐ, e.g., OH, OMe, or OEt. In some embodiments, at least one occurrence of R₅ is —C₁₋₄alkyl-ORₐ, e.g., CH₂OH, CH₂CH₂OH, or CH₂OCH₃. In some embodiments, at least one occurrence of R₅ is alkyl. Non-limiting examples of alkyl include methyl, ethyl, propyl, isopropyl, n-butyl, iso-butyl, sec-butyl, pentyl, hexyl, heptyl, and octyl. In some embodiments, at least one occurrence of R₅ is a cycloalkyl. Non-limiting examples of cycloalkyl include cyclopropyl, cyclobutyl, cyclopentyl, cyclohexyl, and cycloheptyl. In some embodiments, at least one occurrence of R₅ is halogen. Non-limiting examples of halogen include F, Cl, Br, and I. In some embodiments, at least one occurrence of R₅ is halogenated alkyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, at least one occurrence of R₅ is halogenated cycloalkyl. Non-limiting examples of halogenated cycloalkyl includes

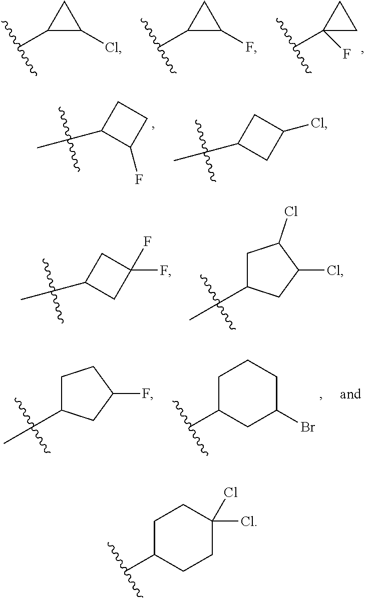

In some embodiments, each occurrence of R₅ independently H, D, CH₃, CH₂CH₃, OH, F, Cl, or Br.

In some embodiments, each occurrence of R₆ is independently H, D, alkyl, halogenated alkyl, cycloalkyl, halogenated cycloalkyl, CN, ORₐ, —C₁₋₄alkyl-ORₐ, or halogen. In some embodiments, each occurrence of R₆ is independently cycloalkyl, halogenated cycloalkyl, or CN. In some embodiments, each occurrence of R₆ is independently H, D, alkyl, halogen, ORₐ, or fluorinated alkyl. In some embodiments, at least one occurrence of R₆ is H or D. In some embodiments, at least one occurrence of R₆ is ORₐ, e.g., OH, OMe, or OEt. In some embodiments, at least one occurrence of R₆ is —C₁₋₄alkyl-ORₐ, e.g., CH₂OH, CH₂CH₂OH, or CH₂OCH₃. In some embodiments, at least one occurrence of R₆ is alkyl. Non-limiting examples of alkyl include methyl, ethyl, propyl, isopropyl, n-butyl, iso-butyl, sec-butyl, pentyl, hexyl, heptyl, and octyl. In some embodiments, at least one occurrence of R₆ is a cycloalkyl. Non-limiting examples of cycloalkyl include cyclopropyl, cyclobutyl, cyclopentyl, cyclohexyl, and cycloheptyl. In some embodiments, at least one occurrence of R₆ is halogen. Non-limiting examples of halogen include F, Cl, Br, and I. In some embodiments, at least one occurrence of R₆ is halogenated alkyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, at least one occurrence of R₆ is halogenated cycloalkyl. Non-limiting examples of halogenated cycloalkyl includes

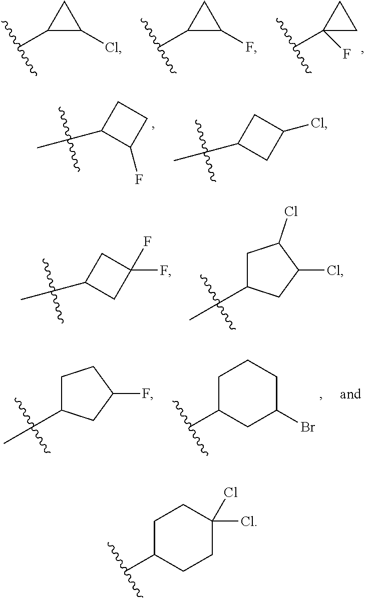

In some embodiments, each occurrence of R₆ independently H, D, CH₃, CH₂CH₃, OH, F, Cl, or Br.

In some embodiments, L₁ is selected from the group consisting of —CH₂—CH₂—, —CH(CH₃)—CH₂—, —CH₂—CH(CH₃)—, —CH₂—C(CH₃)₂—, —CH(OH)—CH₂—, —CH₂—CH(OH)—,

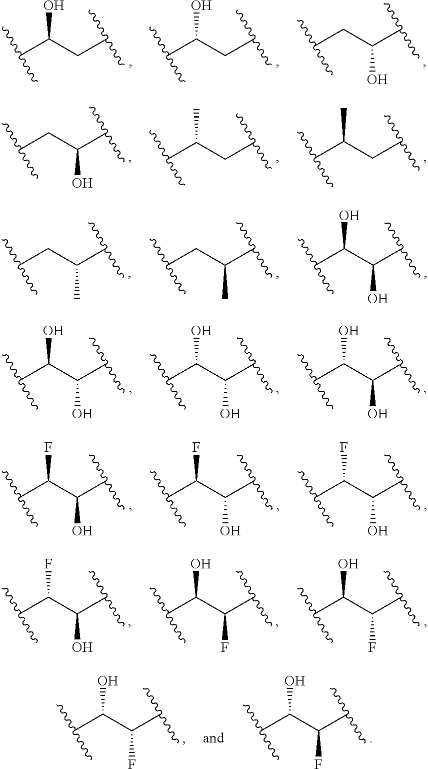

In some embodiments, the structural moiety L₁ is selected from the group consisting of —CH₂—CH₂—,

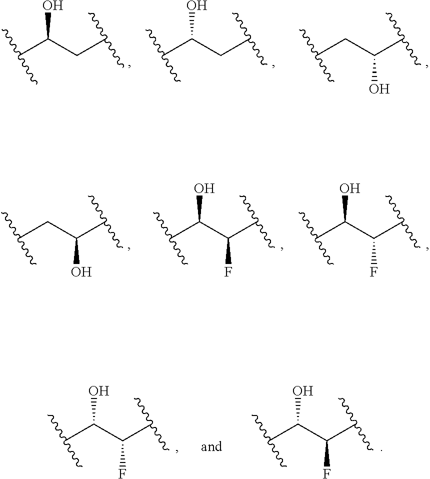

In some embodiment, L₁ is —CH₂—CH₂—,

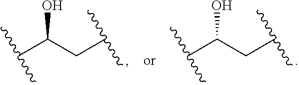 [SMILES: *C[C@@H](*)O ; *C[C@H](*)O]

In some embodiments, L₁ is selected from the list consisting of —CH₂—CH₂—, —CH(CH₃)—CH₂—, —CH₂—CH(CH₃)—, and —CH₂—C(CH₃)₂—. In some embodiment, L₁ is —CH₂—CH₂—. In some embodiments, L₁ is

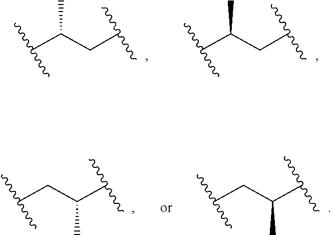

In some embodiments L₁ is

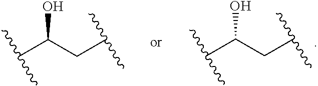 [SMILES: *C[C@@H](*)O ; *C[C@H](*)O]

In some embodiments, L₁ is

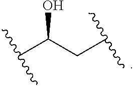 [SMILES: *C[C@@H](*)O]

In some embodiments L₁ is

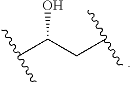 [SMILES: *C[C@H](*)O]

In some embodiments, R₇ is H, D, alkyl, halogenated alkyl, cycloalkyl, halogenated cycloalkyl, CN, or —C₁₋₄alkyl-ORₐ. In some embodiments, R₇ is cycloalkyl, halogenated cycloalkyl, or CN. In some embodiments, R₇ is H, D, alkyl, or fluorinated alkyl. In some embodiments, R₇ is H or D. In some embodiments, at least one occurrence of R₇ is —C₁₋₄alkyl-ORₐ, e.g., CH₂OH, CH₂CH₂OH, or CH₂OCH₃. In some embodiments, R₇ is alkyl. Non-limiting examples of alkyl include methyl, ethyl, propyl, isopropyl, n-butyl, iso-butyl, sec-butyl, pentyl, hexyl, heptyl, and octyl. In some embodiments, R₇ is a cycloalkyl. Non-limiting examples of cycloalkyl include cyclopropyl, cyclobutyl, cyclopentyl, cyclohexyl, and cycloheptyl. In some embodiments, at least one occurrence of R₇ is halogenated alkyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, at least one occurrence of R₇ is halogenated cycloalkyl. Non-limiting examples of halogenated cycloalkyl includes

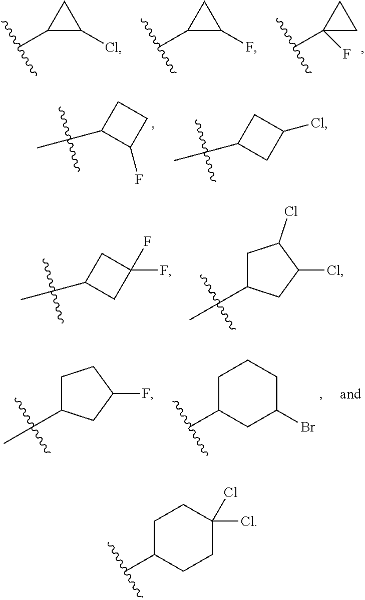

In some embodiments, R₇ is H, CH₃, or CH₂CH₃.

In some embodiments, R₈ is H, D, alkyl, halogenated alkyl, cycloalkyl, halogenated cycloalkyl, CN, or —C₁₋₄alkyl-ORₐ. In some embodiments, R₈ is cycloalkyl, halogenated cycloalkyl, or CN. In some embodiments, R₈ is H, D, alkyl, or fluorinated alkyl. In some embodiments, R₈ is H or D. In some embodiments, at least one occurrence of R₈ is —C₁₋₄alkyl-ORₐ, e.g., CH₂OH, CH₂CH₂OH, or CH₂OCH₃. In some embodiments, R₈ is alkyl. Non-limiting examples of alkyl include methyl, ethyl, propyl, isopropyl, n-butyl, iso-butyl, sec-butyl, pentyl, hexyl, heptyl, and octyl. In some embodiments, R₈ is a cycloalkyl. Non-limiting examples of cycloalkyl include cyclopropyl, cyclobutyl, cyclopentyl, cyclohexyl, and cycloheptyl. In some embodiments, at least one occurrence of R₈ is halogenated alkyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, at least one occurrence of R₈ is halogenated cycloalkyl. Non-limiting examples of halogenated cycloalkyl includes

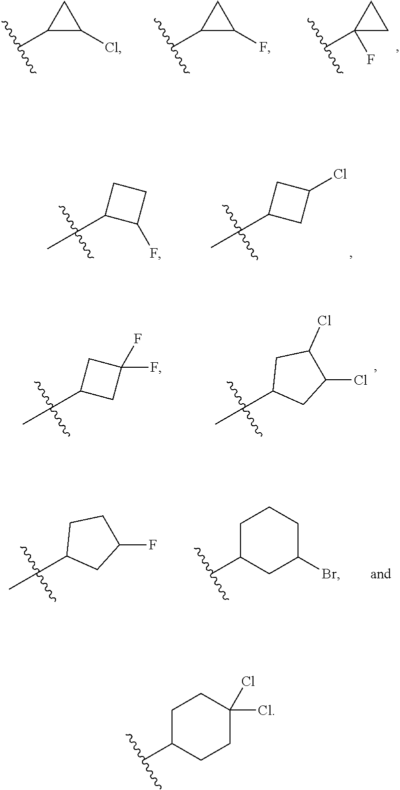

In some embodiments, R₈ is H, CH₃, or CH₂CH₃.

In some embodiments, L₂ is selected from the group consisting of —CH₂—, —CH(CH₃)—, —C(CH₃)₂—, and —CH(CH₂CH₃)—. In some embodiments, the structural moiety L₂ is —CH₂—.

In some embodiments, \textcircled{A} is phenyl which is optionally substituted with by 1-5 substituents each independently selected from the group consisting of H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, and —C₁₋₄alkyl-ORₐ. In some embodiments, \textcircled{A} is phenyl which is optionally substituted with by 1-3 substituents each independently selected from the group consisting of H, D, halogen, alkyl, alkenyl, alkynyl, cycloalkyl, halogenated alkyl, CN, ORₐ, SRₐ, NRₐR_{b}, —C₁₋₄alkyl-SRₐ, and —C₁₋₄alkyl-ORₐ. In some embodiments, \textcircled{A} is phenyl which is optionally substituted with by 1-3 substituents each independently selected from the group consisting of CH₃, CH₂CH₃, OH, F, Cl, Br, OCH₃, CH₂OCH₃, CF₃, CN, C≡CH, and

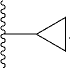 [SMILES: *C1CC1]

In some embodiments, \textcircled{A} is phenyl which is substituted with at least one substituent selected from the group consisting of CH₃, CH₂CH₃, OH, F, Cl, Br, OCH₃, CH₂OCH₃, CF₃, CN, C≡CH, and

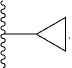 [SMILES: *C1CC1]

In some embodiments, \textcircled{A} is phenyl which is substituted with at least one halogen. In some embodiments, \textcircled{A} is phenyl substituted with one chlorine.

In some embodiments, \textcircled{A} is selected from the group consisting of

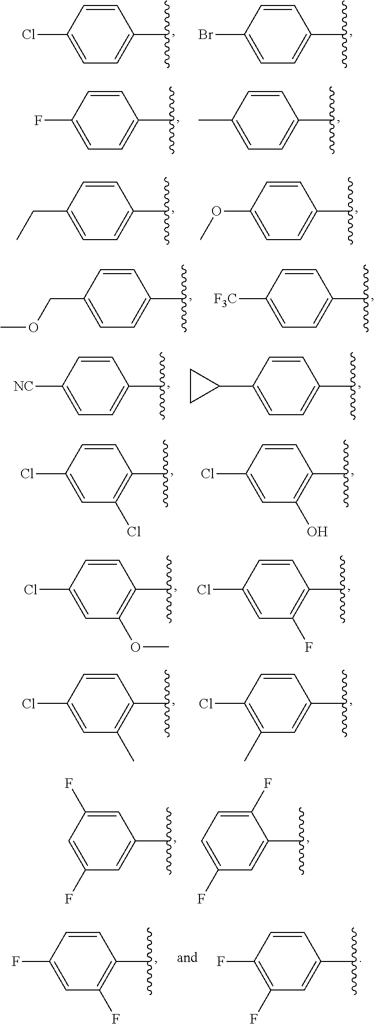

In some embodiments, \textcircled{A} is a 5- or 6-membered heteroaryl which is optionally substituted with by 1-4 substituents each independently selected from the group consisting of H, halogen, alkyl, cycloalkyl, halogenated cycloalkyl, halogenated alkyl, aryl, heteroaryl, CN, ORₐ, SRₐ, NRₐR_{b}, or —C₁₋₄alkyl-ORₐ. In some embodiments, \textcircled{A} is an optionally substituted 5- or 6-membered heteroaryl containing 1-3 heteroatoms each selected from the group consisting of O and S. In further embodiments, \textcircled{A} is an optionally substituted thiophene or furan.

In some embodiments, \textcircled{A} is a 5-membered heteroaryl, wherein the heteroaryl is optionally substituted by alkyl, halogen, OH, or oxo where valence permits. Non-limiting examples of 5-membered heteroaryl include

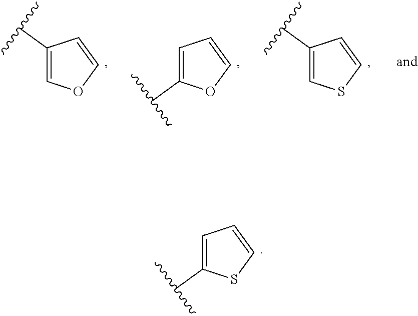 [SMILES: *c1ccoc1 ; *c1ccco1 ; *c1ccsc1 ; *c1cccs1]

In some embodiments, \textcircled{A} is selected from the group consisting of S

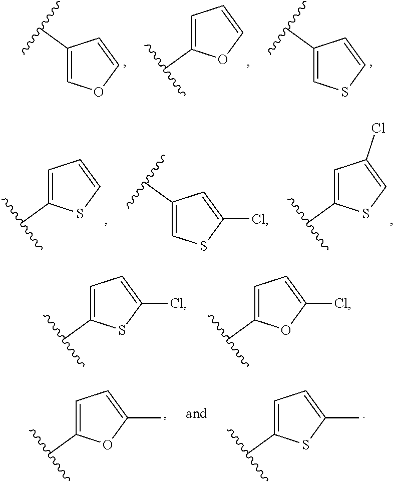

In some embodiments, \textcircled{A} is an optionally substituted 5- or 6-membered heteroaryl or phenyl. In some embodiments, \textcircled{A} is an optionally substituted 5-membered heteroaryl. \textcircled{A} is selected from the group consisting of

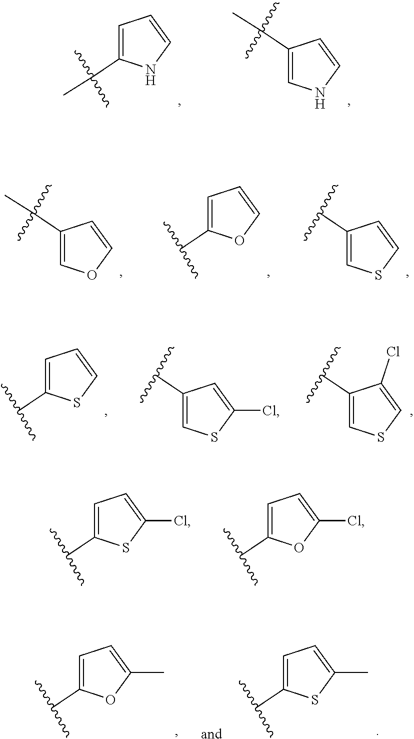

In some specific embodiments, \textcircled{A} is selected from the group consisting of

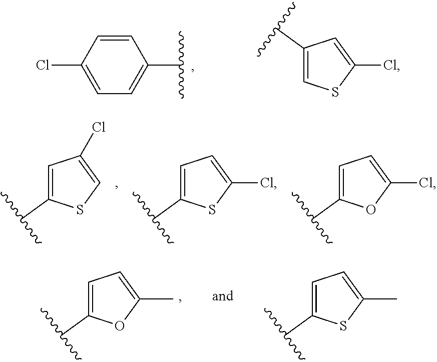

In some embodiments, \textcircled{A} is an optionally substituted 7 to 11 membered bicyclic, or 8 to 16 membered tricyclic aryl or heteroaryl. Non-limiting examples of bicyclic or tricyclic rings include biphenyl, naphthyl, phenanthrenyl, benzothienyl, chromonyl, and coumarinyl.

In some embodiments, \textcircled{A} is selected from the group consisting of optionally substituted

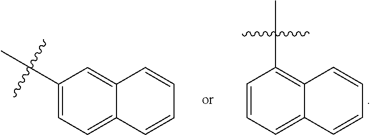 [SMILES: *c1ccc2ccccc2c1 ; *c1cccc2ccccc12]

In some embodiments, R₁ is cycloalkyl, halogenated alkyl, or halogenated cycloalkyl. In some embodiments, R₁ is alkyl or halogenated alkyl. Non-limiting examples of alkyl include methyl, ethyl, propyl, isopropyl, n-butyl, iso-butyl, sec-butyl, pentyl, hexyl, heptyl, and octyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, R₁ is cycloalkyl or halogenated cycloalkyl. Non-limiting examples of cycloalkyl include cyclopropyl, cyclobutyl, cyclopentyl, cyclohexyl, and cycloheptyl. In some embodiments, R₁ is halogen. Non-limiting examples of halogen include F, Cl, Br, and I. In some embodiments, R₁ is halogenated alkyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, R₁ is halogenated cycloalkyl. Non-limiting examples of halogenated cycloalkyl includes

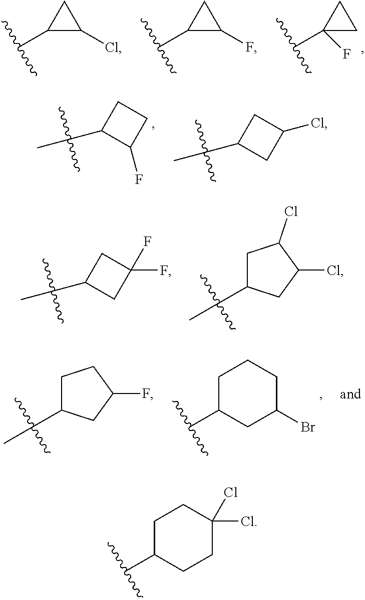

In some embodiments, R₁ is H or D. In some embodiments, R₁ is CN, ORₐ, SRₐ, or NRₐR_{b}. In some embodiments, R₁ is H, D, halogen, alkyl, CN, CF₃, ORₐ, SRₐ, or NRₐR_{b}. In some embodiments, R₁ is selected from the group consisting of H, D, CH₃, CH₂CH₃, OH, F, Cl, Br, OCH₃, CF₃, CN, NH₂, NHCH₃, N(CH₃)₂, and

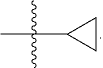 [SMILES: *C1CC1]

In some embodiments, R₁ is selected from the list consisting of H, CH₃, Cl, Br, NH₂, and CF₃.

In some embodiments, R₂ is H, D, halogen, CN, CF₃, ORₐ, SRₐ, NRₐR_{b}, (C═O)NRₐR_{b}, NR_{b}(C═O)Rₐ, (C═O)Rₐ, (C═O)ORₐ, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b}. In some embodiments, R₂ is saturated heterocycle, partially saturated heterocycle, or heteroaryl, each optionally substituted with 1-3 substituents selected from the group consisting of halogen, alkyl, CN, ORₓ, —(CH₂)₁₋₂ORₓ, N(Rₓ)₂, —(CH₂)₁₋₂N(Rₓ)₂, (C═O)Rₓ, (C═O)N(Rₓ)₂, NRₓ(C═O)Rₓ, and oxo where valence permits. In some embodiments, R₂ is alkyl, alkenyl, or alkynyl, each optionally substituted with 1-3 substituents selected from the group consisting of halogen, CN, ORₓ, —(CH₂)₁₋₂ORₓ, N(Rₓ)₂, —(CH₂)₁₋₂N(Rₓ)₂, (C═O)Rₓ, (C═O)N(Rₓ)₂, NRₓ(C═O)Rₓ, and oxo where valence permits. In some embodiments, R₂ is cycloalkyl, aryl, or alkylaryl, alkylheteroaryl.

In some embodiments, R₂ is H, D, or alkyl, wherein the alkyl is optionally substituted by OH, oxo, or NH₂. Non-limiting examples of alkyl include methyl, ethyl, propyl, isopropyl, n-butyl, iso-butyl, sec-butyl, pentyl, hexyl, heptyl, and octyl. In some embodiments, R₂ is alkenyl or alkynyl, wherein the alkenyl and alkynyl are optionally substituted by OH, oxo, or NH₂. Non-limiting examples of alkenyl include ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. Non-limiting examples of alkynyl include ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. In some embodiments, R₂ is a cycloalkyl. Non-limiting examples of cycloalkyl include cyclopropyl, cyclobutyl, cyclopentyl, cyclohexyl, and cycloheptyl. In some embodiments, R₂ is halogen. Non-limiting examples of halogen include F, Cl, Br, and I. In some embodiments, R₂ is halogenated alkyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, R₂ is halogenated cycloalkyl. Non-limiting examples of halogenated cycloalkyl includes

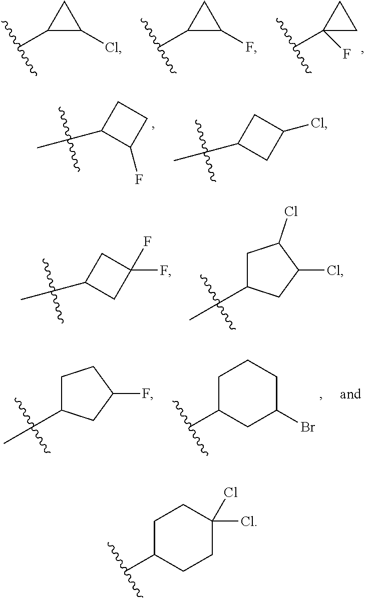

In some embodiments, R₂ is ORₐ, SRₐ, NRₐR_{b}, (C═O)NRₐR_{b}, NR_{b}(C═O)Rₐ, (C═O)Rₐ, (C═O)ORₐ, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b}. In some embodiments, R₂ is ORₐ, SRₐ, or NRₐR_{b}. In some embodiments, R₂ is (C═O)NRₐR_{b}, NR_{b}(C═O)Rₐ, (C═O)Rₐ, or (C═O)ORₐ. In some embodiments, R₂ is —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, or —C₁₋₄alkyl-NRₐCOR_{b}. In some embodiments, R₂ is O—C₁₋₄alkyl-Rₐ or NRₐ—C₁₋₄alkyl-R_{b}.

In some specific embodiments, R₂ is NH₂, CH₂NH₂, or CH₂CH₂NH₂. In other specific embodiments, R₂ is OH, CH₂OH, or CH₂CH₂OH.

In still other embodiments, R₂ is an optionally substituted 4-, 5-, 6- or 7-membered heterocycle, partially saturated heterocycle, or heteroaryl, each containing 1-3 heteroatoms each selected from the group consisting of N, O, and S. In further embodiments, R₂ is selected from the group consisting of

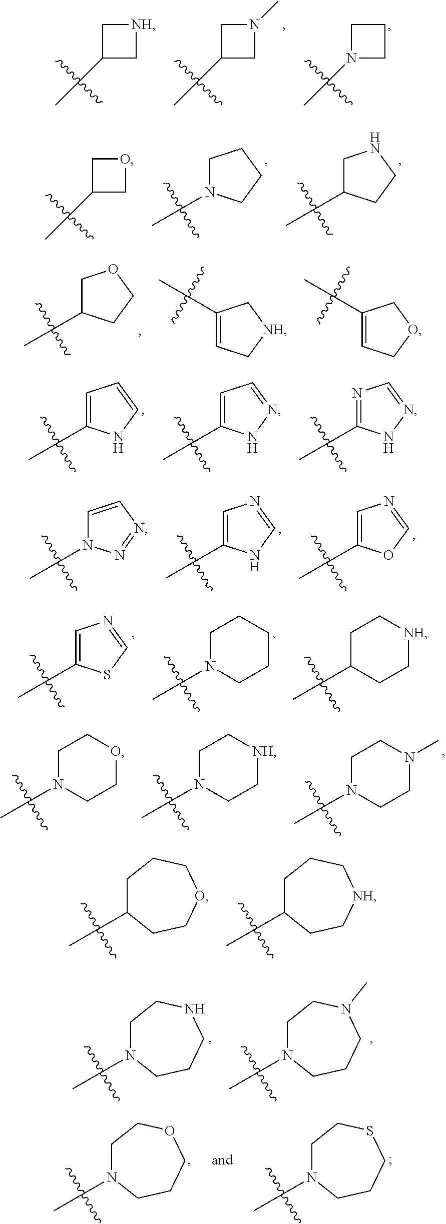

wherein each is optionally substituted by alkyl, OH, NH₂, or oxo where valence permits. In some embodiments, R₂ is a N-containing heterocycle, partially saturated heterocycle, or heteroaryl, wherein each is optionally substituted by alkyl, OH, NH₂, or oxo where valence permits. Non-limiting examples of N-containing heterocycle partially saturated heterocycle, and heteroaryl include

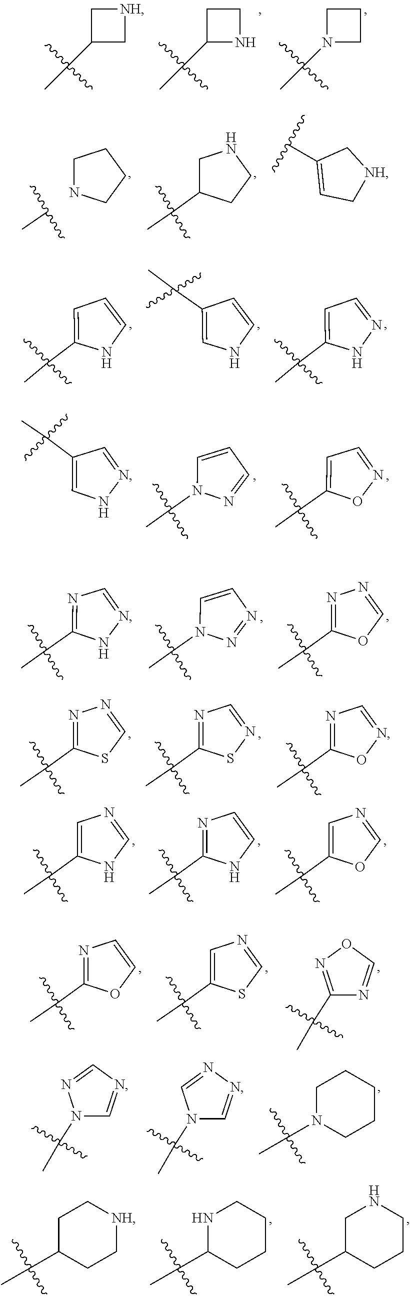

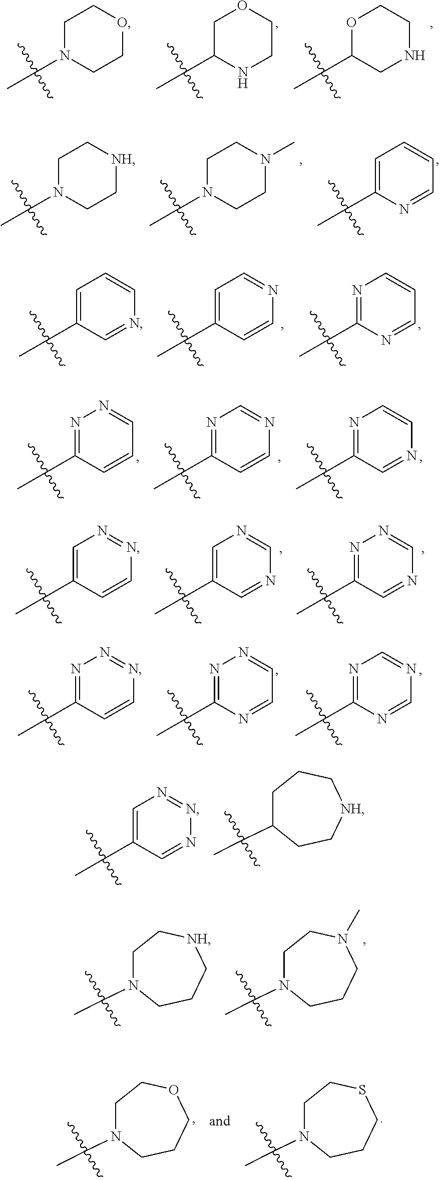

In some embodiments, R₂ is selected from the group consisting of H, D, CH₃, CH₂CH₃, OH, F, Cl, Br, I, OCH₃, CF₃, CN, NH₂, NHCH₃, N(CH₃)₂, CH═CH₂,

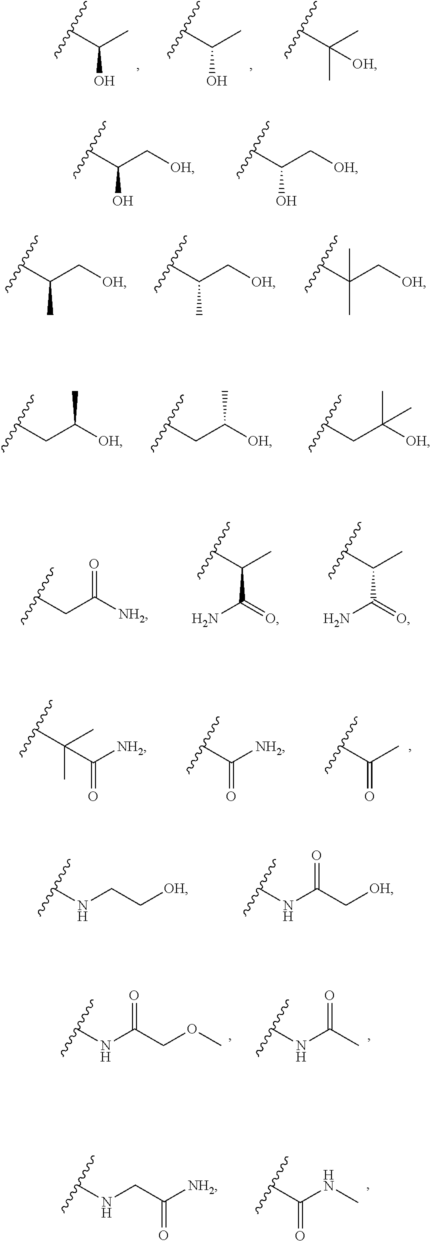

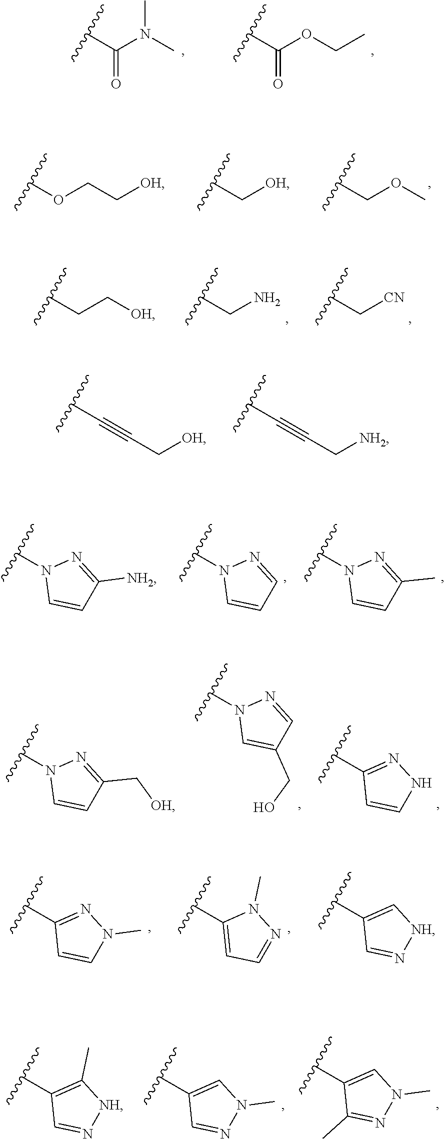

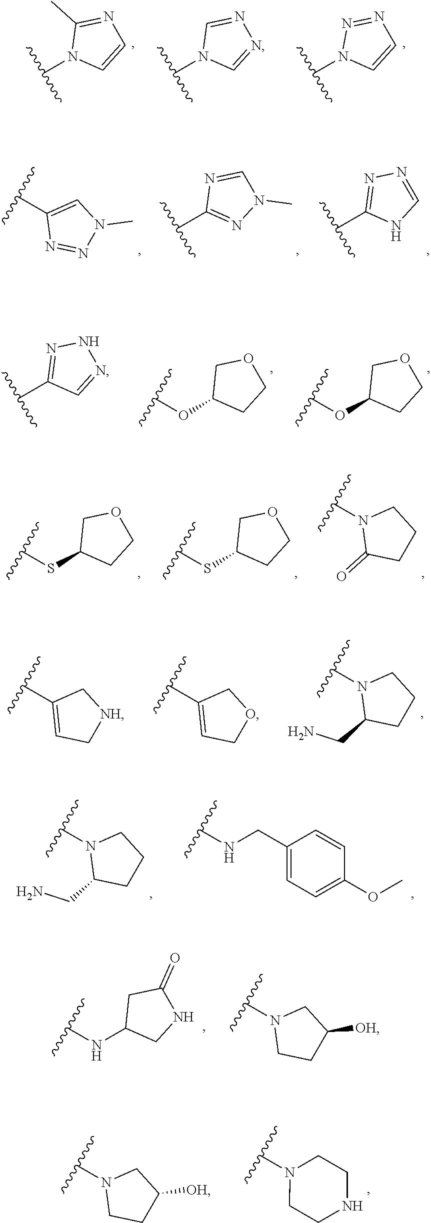

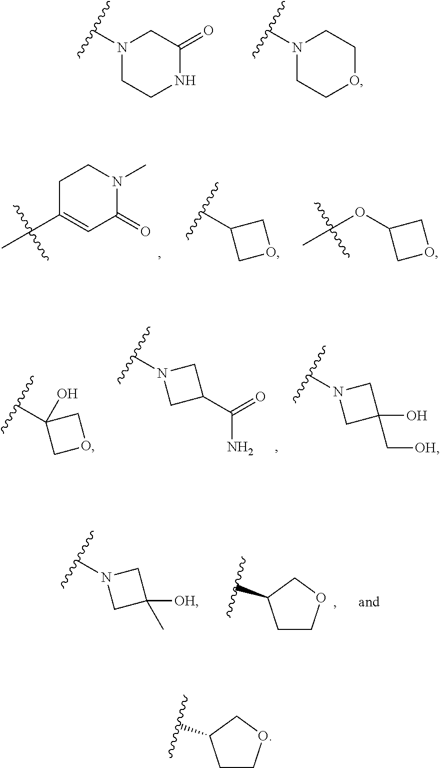

In some embodiments, R₂ is selected from the group consisting of H, CH₃, Cl, OCH₃, CH₂OH, CN, CH₂CN, NH₂,

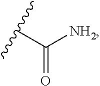 [SMILES: *C(N)=O]

CH₂NH₂, CH═CH₂,

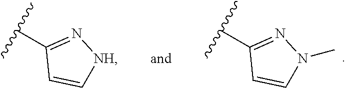

In some embodiments, R₃ is H, D, halogen, alkyl, halogenated alkyl, heteroaryl, or CN. In some embodiments, R₃ is ORₐ, SRₐ, NRₐR_{b}, (C═O)NRₐR_{b}, —C₁₋₄alkyl-ORₐ, —C₁₋₄alkyl-SRₐ, —C₁₋₄alkyl-NRₐR_{b}, or —C₁₋₄alkyl-CONRₐR_{b}. In some embodiments, R₃ is alkenyl, alkynyl, cycloalkyl, saturated heterocycle, partially saturated heterocycle, aryl, alkylaryl, alkylheteroaryl, NR_{b}(C═O)Rₐ, (C═O)Rₐ, (C═O)ORₐ, —C₁₋₄alkyl-COORₐ, —C₁₋₄alkyl-NRₐCOR_{b}, O—C₁₋₄alkyl-Rₐ, or NRₐ—C₁₋₄alkyl-R_{b}.

In some embodiments, R₃ is H, D, or alkyl, wherein the alkyl is optionally substituted by OH, oxo, or NH₂. Non-limiting examples of alkyl include methyl, ethyl, propyl, isopropyl, n-butyl, iso-butyl, sec-butyl, pentyl, hexyl, heptyl, and octyl. In some embodiments, R₃ is alkenyl or alkynyl, wherein the alkenyl and alkynyl are optionally substituted by OH, oxo, or NH₂. Non-limiting examples of alkenyl include ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. Non-limiting examples of alkynyl include ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. In some embodiments, R₃ is a cycloalkyl. Non-limiting examples of cycloalkyl include cyclopropyl, cyclobutyl, cyclopentyl, cyclohexyl, and cycloheptyl. In some embodiments, R₃ is halogen. Non-limiting examples of halogen include F, Cl, Br, and I. In some embodiments, R₃ is halogenated alkyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, R₃ is halogenated cycloalkyl. Non-limiting examples of halogenated cycloalkyl includes

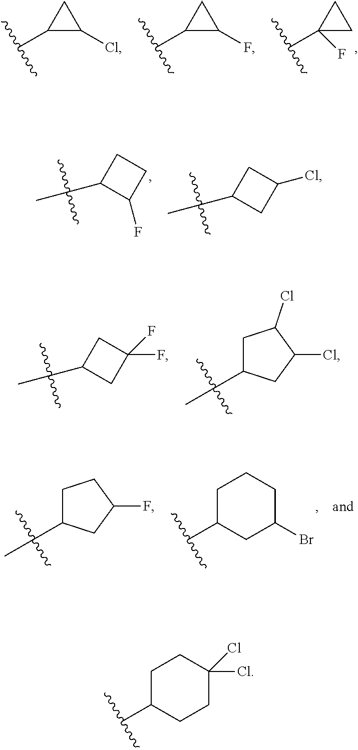

In some embodiments, R₃ is ORₐ, SRₐ, NRₐR_{b}, (C═O)NRₐR_{b}, NR_{b}(C═O)Rₐ, (C═O)Rₐ, (C═O)ORₐ, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b}. In some embodiments, R₃ is ORₐ, SRₐ, NRₐR_{b}, (C═O)NRₐR_{b}, —C₁₋₄alkyl-CN, —C₁₋₄alkyl-ORₐ, —C₁₋₄alkyl-SRₐ, —C₁₋₄alkyl-NRₐR_{b}, or —C₁₋₄alkyl-CONRₐR_{b}. In some embodiments, R₃ is ORₐ, SRₐ, or NRₐR_{b}. In some embodiments, R₃ is (C═O)NRₐR_{b}, NR_{b}(C═O)Rₐ, (C═O)Rₐ, or (C═O)ORₐ. In some embodiments, R₃ is —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, or —C₁₋₄alkyl-NRₐCOR_{b}. In some embodiments, R₃ is O—C₁₋₄alkyl-Rₐ or NRₐ—C₁₋₄alkyl-R_{b}.

In some specific embodiments, R₃ is NH₂, CH₂NH₂, or CH₂CH₂NH₂. In other specific embodiments, R₃ is OH, CH₂OH, or CH₂CH₂OH.

In some specific embodiments, R₃ is alkenyl, alkynyl, cycloalkyl, saturated heterocycle, partially saturated heterocycle, aryl, alkylaryl, alkylheteroaryl, NR_{b}(C═O)Rₐ, (C═O)Rₐ, (C═O)ORₐ, —C₁₋₄alkyl-COORₐ, —C₁₋₄alkyl-NRₐCOR_{b}, O—C₁₋₄alkyl-Rₐ, or NRₐ—C₁₋₄alkyl-R_{b}.

In still other embodiments, R₃ is an optionally substituted 4-, 5-, 6- or 7-membered heterocycle, partially saturated heterocycle, or heteroaryl, each containing 1-3 heteroatoms each selected from the group consisting of N, O, and S. In further embodiments, R₃ is selected from the group consisting of

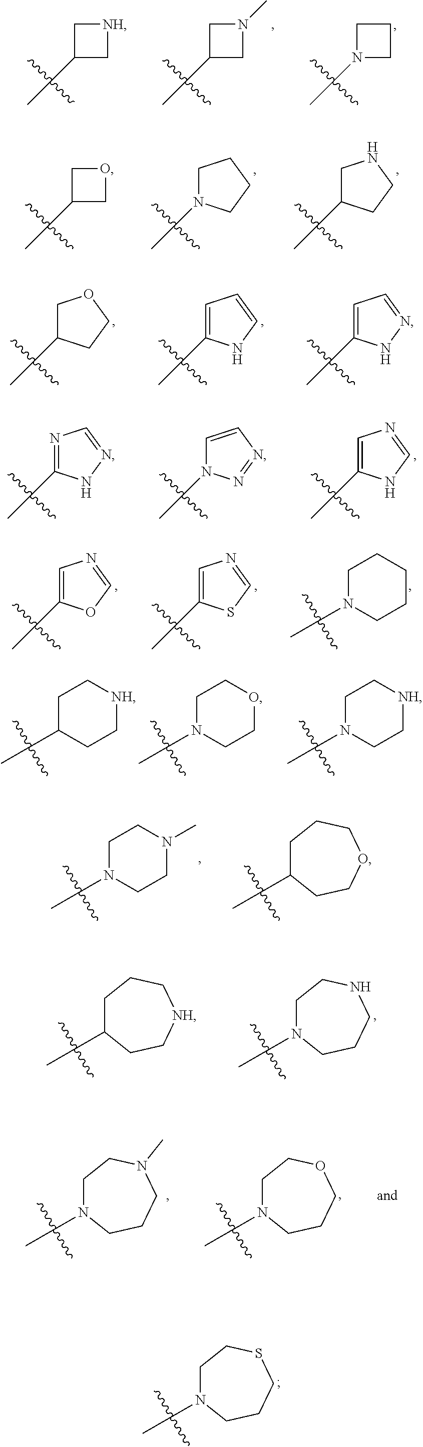

wherein each is optionally substituted by alkyl, OH, NH₂, or oxo where valence permits. In some embodiments, R₃ is a N-containing heterocycle, partially saturated heterocycle, or heteroaryl, wherein each is optionally substituted by alkyl, OH, NH₂, or oxo where valence permits. Non-limiting examples of N-containing heterocycle partially saturated heterocycle, and heteroaryl include

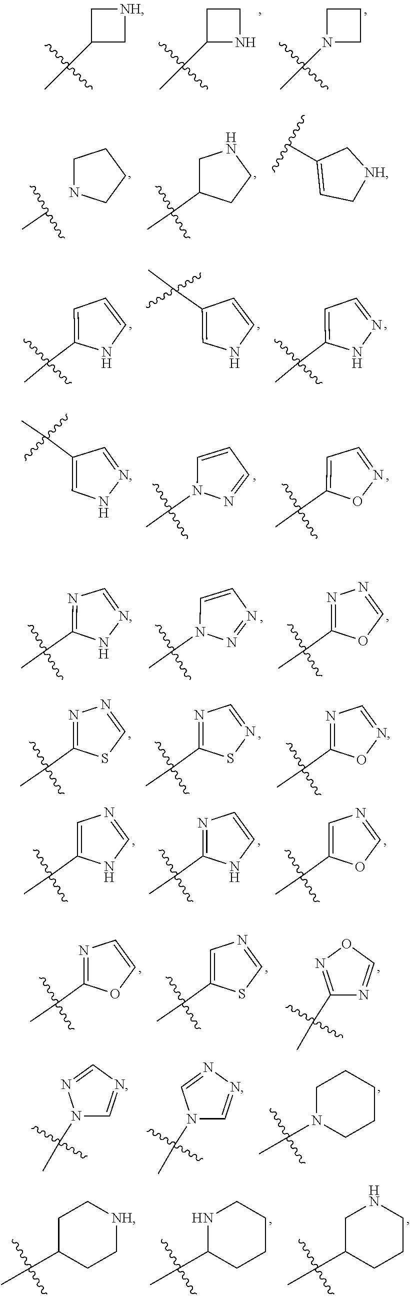

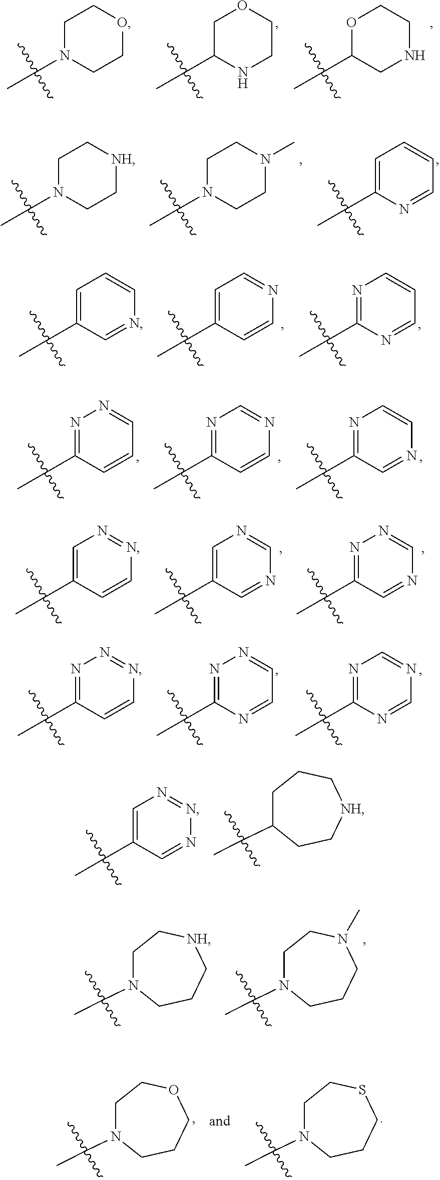

In some embodiments, R₃ is selected from the group consisting of H, D, CH₃, CH₂CH₃, OH F, Cl, Br, OCH₃, CF₃, CN, CH₂CN, CH═CH₂, NH₂, NHCH₃, N(CH₃)₂,

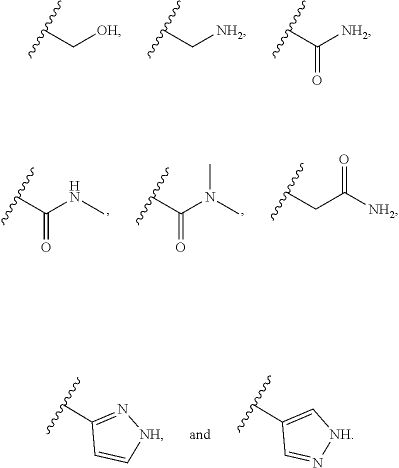

In some embodiments, R₃ is selected from the group consisting of H, NH₂, CH₃, CN, Cl, Br,

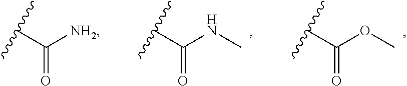

CH₂CN, CH₂OH,

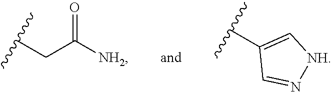 [SMILES: *CC(N)=O ; "and" ; *c1cn[nH]c1]

In some embodiments, R₄ is aryl, heteroaryl. In some embodiments, R₄ is alkyl or halogenated alkyl. Non-limiting examples of alkyl include methyl, ethyl, propyl, isopropyl, n-butyl, iso-butyl, sec-butyl, pentyl, hexyl, heptyl, and octyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, R₄ is cycloalkyl or halogenated cycloalkyl. Non-limiting examples of cycloalkyl include cyclopropyl, cyclobutyl, cyclopentyl, cyclohexyl, and cycloheptyl. In some embodiments, R₄ is halogen. Non-limiting examples of halogen include F, Cl, Br, and I. In some embodiments, R₄ is halogenated alkyl. Non-limiting examples of halogenated alkyl include CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, and CHClCHClCH₃. In some embodiments, R₄ is halogenated cycloalkyl. Non-limiting examples of halogenated cycloalkyl includes

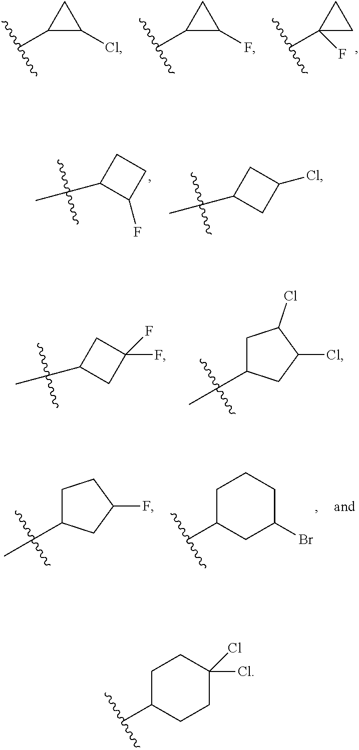

In some embodiments, R₄ is H or D. In some embodiments, R₄ is CN, ORₐ, SRₐ, or NRₐR_{b}. In some embodiments, R₄ is H, D, halogen, alkyl, CN, CF₃, ORₐ, SRₐ, —C₁₋₄alkyl-ORₐ, or NRₐR_{b}. In some embodiments, R₄ is selected from the group consisting of H, D, CH₃, CH₂CH₃, OH, F, Cl, Br, OCH₃, CF₃, CN, NH₂, NHCH₃, N(CH₃)₂, CH₂OH,

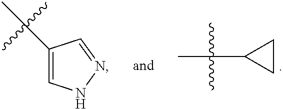

In some embodiments, R₄ is selected from the group consisting of H, D, CH₃, CH₂CH₃, OH, F, Cl, Br, OCH₃, CF₃, CN, NH₂, NHCH₃, N(CH₃)₂, CH₂OH, and

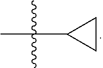 [SMILES: *C1CC1]

In some embodiments, R₄ is selected from the group consisting of H, CH₃, NH₂, CH₂OH, F, Br, and CN.

In some embodiments, at least one occurrence of Rₐ or R_{b} is independently H, D, Me, Et, Pr, CH₂CH₂OH, phenyl, or a heterocycle. In some embodiments, at least one occurrence of Rₐ or R_{b} is independently H, alkyl, alkenyl, cycloalkyl, saturated heterocycle, aryl, or heteroaryl. In some embodiments, at least one occurrence of Rₐ or R_{b} is independently H, alkyl or alkenyl. In some embodiments, at least one occurrence of Rₐ or R_{b} is independently H, Me, Et, Pr, or Bu. In some embodiments, at least one occurrence of Rₐ or R_{b} is independently (C═O)Rₓ, (C═O)N(Rₓ)₂, SO₂Rₓ, NRₓ(C═O)NRₓ₂, or (C═O)Rₓ. In some embodiments, at least one occurrence of Rₐ or R_{b} is independently a heterocycle selected from the group consisting of

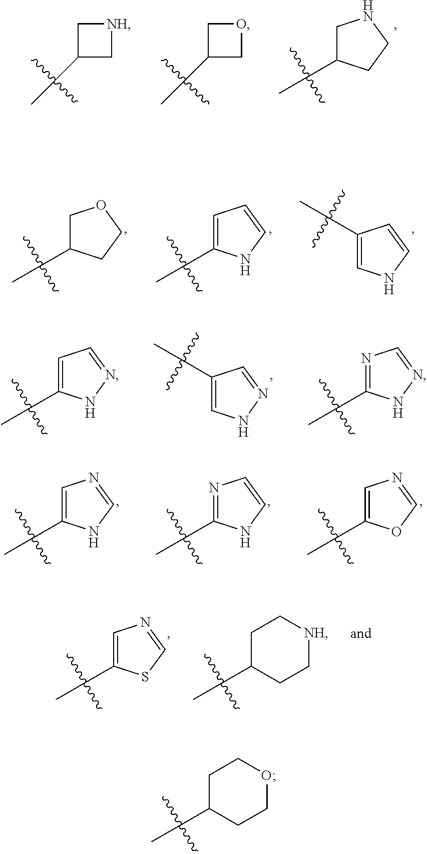

where the heterocycle is optionally substituted by alkyl, OH, oxo, or (C═O)C₁₋₄alkyl where valence permits. In some embodiments, at least one occurrence of Rₐ or R_{b} is H, Me, phenyl,

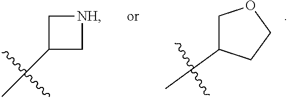 [SMILES: *C1CNC1 ; *C1CCOC1]

In some embodiments, at least one occurrence of Rₐ or R_{b} is independently H or

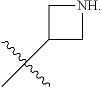 [SMILES: *C1CNC1]

In some embodiments, Rₐ and R_{b} together with the nitrogen atom that they are connected to form an optionally substituted heterocycle comprising the nitrogen atom and 0-3 additional heteroatoms each selected from the group consisting of N, O, and S. In some embodiments, Rₐ and R_{b} together with the carbon atom that they are connected to form a cycloalkyl, optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. Non-limiting examples of cycloalkyl include cyclopropyl, cyclobutyl, cyclopentyl, cyclohexyl, and cycloheptyl. In some embodiments, Rₐ and R_{b} together with the nitrogen atom that they are connected to form an optionally substituted heterocycle including the nitrogen atom and 0-3 additional heteroatoms each selected from the group consisting of N, O, and S, optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. Non-limiting examples of heterocycle include

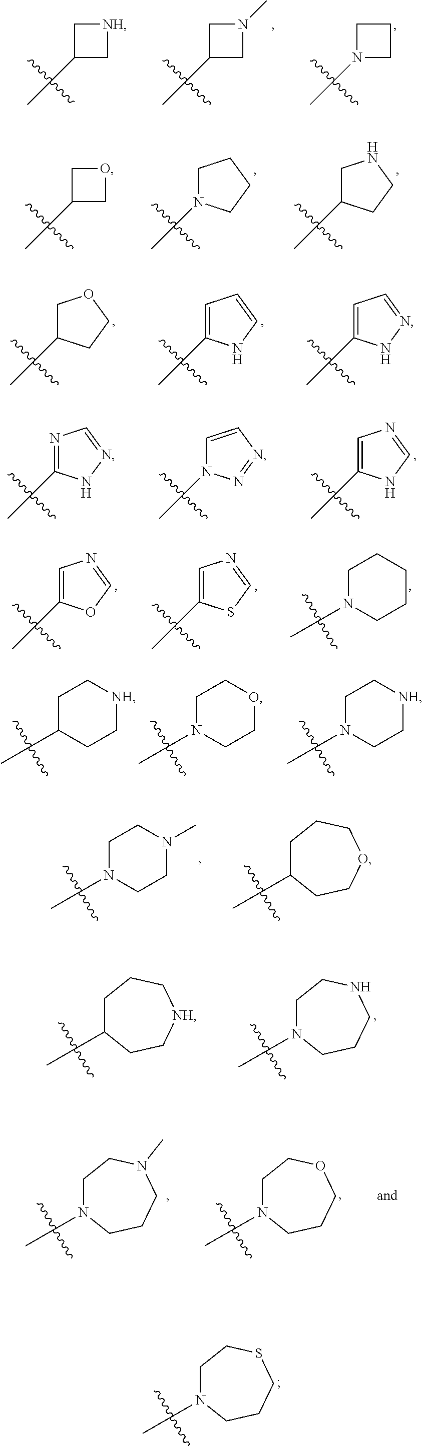

In some embodiments, the alkyl, halogenated alkyl, cycloalkyl, halogenated cycloalkyl, and heterocycle in R₁ are optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. In some embodiments, the alkyl, alkenyl, alkynyl, cycloalkyl, halogenated alkyl, halogenated alkenyl, halogenated alkynyl, halogenated cycloalkyl, saturated heterocycle, partially saturated heterocycle, aryl, heteroaryl, alkylaryl, and alkylheteroaryl in R₂ are optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. In some embodiments, the alkyl, alkenyl, alkynyl, cycloalkyl, halogenated alkyl, halogenated alkenyl, halogenated alkynyl, halogenated cycloalkyl, saturated heterocycle, partially saturated heterocycle, aryl, heteroaryl, alkylaryl, and alkylheteroaryl in R₃ are optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. In some embodiments, the alkyl, halogenated alkyl, cycloalkyl, halogenated cycloalkyl, and heterocycle in R₄ are optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. In some embodiments, the alkyl, halogenated alkyl, cycloalkyl, and halogenated cycloalkyl in R₅ is optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. In some embodiments, the alkyl, halogenated alkyl, cycloalkyl, and halogenated cycloalkyl in R₆ is optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. In some embodiments, the alkyl, halogenated alkyl, cycloalkyl, and halogenated cycloalkyl in R₇ is optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. In some embodiments, the alkyl, halogenated alkyl, cycloalkyl, and halogenated cycloalkyl in R₈ is optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits. In some embodiments, the alkyl, cycloalkyl, halogenated alkyl, heteroalkyl, halogenated heteroalkyl, halogenated cycloalkyl, and saturated heterocycle in Rₐ and R_{b} are optionally substituted by 1-4 substituents each independently selected from the group consisting of alkyl, cycloalkyl, halogenated cycloalkyl, halogenated alkyl, halogen, CN, ORₓ, —(CH₂)₀₋₂ORₓ, N(Rₓ)₂, (C═O)Rₓ, (C═O)N(Rₓ)₂, NRₓ(C═O)Rₓ, and oxo where valence permits.

In some embodiments, each occurrence of Rₓ is independently H, alkyl, or heterocycle optionally substituted by alkyl, OH, or alkoxy. In some embodiments, each occurrence of Rₓ is independently H or alkyl. In some embodiments, each occurrence of Rₓ is substituted heterocycle. In some embodiments, the two Rₓ groups together with the nitrogen atom that they are connected to form an optionally substituted heterocycle including the nitrogen atom and 0-3 additional heteroatoms each selected from the group consisting of N, O, and S. In some specific embodiments, each occurrence of Rₓ is independently H or Me.

In some embodiments,

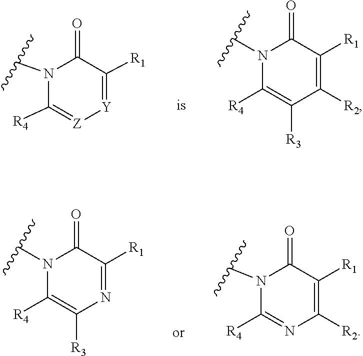

In some embodiments, the compound has the structure of Formula Ia, Ib, or Ic:

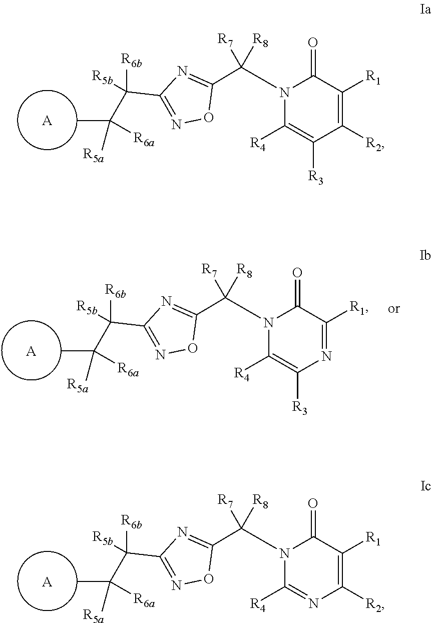

wherein
each occurrence of R₅ₐ is independently H, D, alkyl, halogen, ORₐ, or fluorinated alkyl;
each occurrence of R_{5b} is independently H, D, alkyl, halogen, ORₐ, or fluorinated alkyl;
each occurrence of R₆ₐ is independently H, D, alkyl, halogen, ORₐ, or fluorinated alkyl; and
each occurrence of R_{6b} is independently H, D, alkyl, halogen, ORₐ, or fluorinated alkyl.

In some embodiments, \textcircled{A}, R₁, R₂, R₃, R₄, R₇, and R₈ in Formula Ia, Ib, and Ic are as defined above for the compound of Formula I. Other substituents are defined herein.

In some embodiments, at least one occurrence of R₅ₐ is H or D. In some embodiments, at least one occurrence of R₅ₐ is ORₐ, e.g., OH or OMe. In some embodiments, at least one occurrence of R₅ₐ is alkyl, e.g., methyl, ethyl, propyl, isopropyl, n-butyl, iso-butyl, sec-butyl, pentyl, hexyl, heptyl, or octyl. In some embodiments, at least one occurrence of R₅ₐ is halogen, e.g., F, Cl, Br, or I. In some embodiments, at least one occurrence of R₅ₐ is fluorinated alkyl, e.g., CF₃, CH₂F, CHF₂, CH₂Cl, CH₂CF₃, CHFCH₃, CF₂CH₃, or CH₂CHF₂.

In some embodiments, at least one occurrence of R_{5b} is H or D. In some embodiments, at least one occurrence of R_{5b} is ORₐ, e.g., OH or OMe. In some embodiments, at least one occurrence of R_{5b} is alkyl, e.g., methyl, ethyl, propyl, isopropyl, n-butyl, iso-butyl, sec-butyl, pentyl, hexyl, heptyl, or octyl. In some embodiments, at least one occurrence of R_{5b} is halogen, e.g., F, Cl, Br, or I. In some embodiments, at least one occurrence of R_{5b} is fluorinated alkyl, e.g., CF₃, CH₂F, CHF₂, CH₂Cl, CH₂CF₃, CHFCH₃, CF₂CH₃, or CH₂CHF₂.

In some embodiments, at least one occurrence of R₆ₐ is H or D. In some embodiments, at least one occurrence of R₆ₐ is ORₐ, e.g., OH or OMe. In some embodiments, at least one occurrence of R₆ₐ is alkyl, e.g., methyl, ethyl, propyl, isopropyl, n-butyl, iso-butyl, sec-butyl, pentyl, hexyl, heptyl, or octyl. In some embodiments, at least one occurrence of R₆ₐ is halogen, e.g., F, Cl, Br, or I. In some embodiments, at least one occurrence of R₆ₐ is fluorinated alkyl, e.g., CF₃, CH₂F, CHF₂, CH₂Cl, CH₂CF₃, CHFCH₃, CF₂CH₃, or CH₂CHF₂.

In some embodiments, at least one occurrence of R_{6b} is H or D. In some embodiments, at least one occurrence of R_{6b} is ORₐ, e.g., OH or OMe. In some embodiments, at least one occurrence of R_{6b} is alkyl, e.g., methyl, ethyl, propyl, isopropyl, n-butyl, iso-butyl, sec-butyl, pentyl, hexyl, heptyl, or octyl. In some embodiments, at least one occurrence of R_{6b} is halogen, F, Cl, Br, or I. In some embodiments, at least one occurrence of R_{6b} is fluorinated alkyl, e.g., CF₃, CH₂F, CHF₂, CH₂Cl, CH₂CF₃, CHFCH₃, CF₂CH₃, or CH₂CHF₂.

In some embodiments, the compound has a structure of Formula IIa, IIb, or IIc:

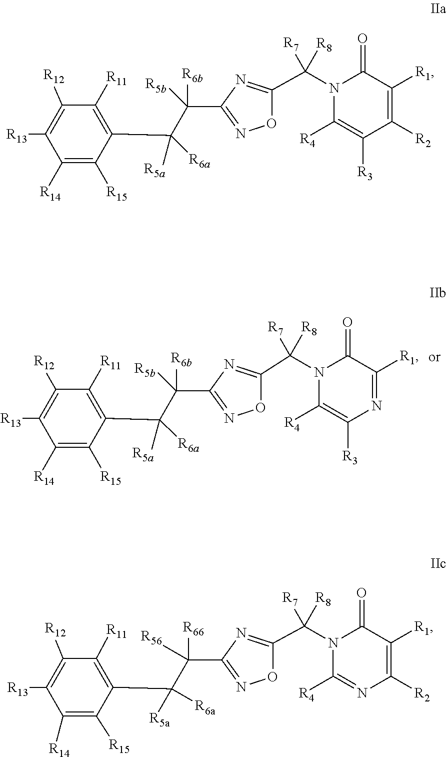

wherein
each occurrence of R₅ₐ is independently H, D, alkyl, halogen, ORₐ, or fluorinated alkyl;
each occurrence of R_{5b} is independently H, D, alkyl, halogen, ORₐ, or fluorinated alkyl;
each occurrence of R₆ₐ is independently H, D, alkyl, halogen, ORₐ, or fluorinated alkyl;
each occurrence of R_{6b} is independently H, D, alkyl, halogen, ORₐ, or fluorinated alkyl;
each occurrence of R₁₁ is independently H, D, halogen, alkyl, cycloalkyl, halogenated cycloalkyl, halogenated alkyl, alkenyl, alkynyl, aryl, heteroaryl, CN, ORₐ, SRₐ, NRₐR_{b}, —C₁₋₄alkyl-SRₐ, or —C₁₋₄alkyl-ORₐ;
each occurrence of R₁₂ is independently H, D, halogen, alkyl, cycloalkyl, halogenated cycloalkyl, halogenated alkyl, alkenyl, alkynyl, aryl, heteroaryl, CN, ORₐ, SRₐ, NRₐR_{b}, —C₁₋₄alkyl-SRₐ, or —C₁₋₄alkyl-ORₐ;
each occurrence of R₁₃ is independently H, D, halogen, alkyl, cycloalkyl, halogenated cycloalkyl, halogenated alkyl, alkenyl, alkynyl, aryl, heteroaryl, CN, ORₐ, SRₐ, NRₐR_{b}, —C₁₋₄alkyl-SRₐ, or —C₁₋₄alkyl-ORₐ;
each occurrence of R₁₄ is independently H, D, halogen, alkyl, cycloalkyl, halogenated cycloalkyl, halogenated alkyl, alkenyl, alkynyl, aryl, heteroaryl, CN, ORₐ, SRₐ, NRₐR_{b}, —C₁₋₄alkyl-SRₐ, or —C₁₋₄alkyl-ORₐ; and
each occurrence of R₁₅ is independently H, D, halogen, alkyl, cycloalkyl, halogenated cycloalkyl, halogenated alkyl, alkenyl, alkynyl, aryl, heteroaryl, CN, ORₐ, SRₐ, NRₐR_{b}, —C₁₋₄alkyl-SRₐ, or —C₁₋₄alkyl-ORₐ.

In some embodiments, R₁, R₂, R₃, R₄, R₇, and R₈ in Formula IIa, IIb and IIc are as defined above for the compound of Formula I. Other substituents are defined herein.

In some embodiments, at least one of R₁₁, R₁₂, R₁₃, R₁₄, and R₁₅ is not H. In some embodiments, at least two of R₁₁, R₁₂, R₁₃, R₁₄, and R₁₅ are not H.

In some embodiments, at least one of R₁₁, R₁₂, R₁₃, R₁₄, and R₁₅ is H, alkyl, CF₃, or halogen. In some embodiments, at least one of R₁₁, R₁₂, R₁₃, R₁₄, and R₁₅ is CN, CF₃, OCF₃, ORₐ, or SRₐ. In some embodiments, at least one of R₁₁, R₁₂, R₁₃, R₁₄, and R₁₅ is halogen, NRₐR_{b}, —C₁₋₄alkyl-SRₐ, or —C₁₋₄alkyl-ORₐ. In some embodiments, at least one of R₁₁, R₁₂, R₁₃, R₁₄, and R₁₅ is ORₐ, SRₐ, or NRₐR_{b}. In some embodiments, at least one of R₁₁, R₁₂, R₁₃, R₁₄, and R₁₅ is H, halogen, fluorinated alkyl, alkyl, alkenyl, or alkynyl. In some embodiments, at least one of R₁₁, R₁₂, R₁₃, R₁₄, and R₁₅ is CH₃, CH₂CH₃, OH, F, Cl, Br, OCH₃, CH₂OCH₃, CF₃, CN, C≡CH, or

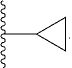 [SMILES: *C1CC1]

In some embodiments, at least one of R₁₁, R₁₂, R₁₃, R₁₄, and R₁₅ is H, Me, Et, i-Pr, n-Bu, CF₂H, CF₂Cl, or CF₃. In some embodiments, at least one of R₁₁, R₁₂, R₁₃, R₁₄, and R₁₅ is OH, OCH₃, CH₂OCH₃. In some embodiments, at least one of R₁₁, R₁₂, R₁₃, R₁₄, and R₁₅ is halogen, e.g., Cl, F, Br, or I. In some embodiments, at least one of R₁₁, R₁₂, R₁₃, R₁₄, and R₁₅ is Cl. In some embodiments, at least one of R₁₁, R₁₂, R₁₃, R₁₄, and R₁₅ is halogenated alkyl, e.g., CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, or CHClCHClCH₃. In some embodiments, at least one of R₁₁, R₁₂, R₁₃, R₁₄, and R₁₅ is halogenated cycloalkyl, e.g.,

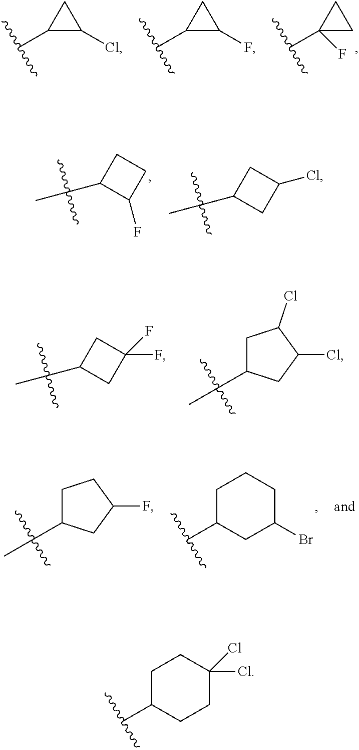

In some embodiments, at least one of R₁₁, R₁₂, R₁₃, R₁₄, and R₁₅ is alkenyl, e.g., ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, or (E)-pent-1-enyl. In some embodiments, at least one of R₁₁, R₁₂, R₁₃, R₁₄, and R₁₅ is alkynyl, e.g., ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. In some embodiments, at least one of R₁₁, R₁₂, R₁₃, R₁₄, and R₁₅ is CN. In some embodiments, at least two of R₁₁, R₁₂, R₁₃, R₁₄, and R₁₅ are independently selected from the group consisting of CH₃, CH₂CH₃, OH, F, Cl, Br, OCH₃, CH₂OCH₃, CF₃, CN, C≡CH, or

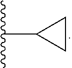 [SMILES: *C1CC1]

In some embodiments, R₁₁, R₁₂, R₁₄, and R₁₅ are H; and R₁₃ is H, D, halogen, alkyl, cycloalkyl, CN, CF₃, ORₐ, SRₐ, NRₐR_{b}, —C₁₋₄alkyl-SRₐ, or —C₁₋₄alkyl-ORₐ. In some embodiments, R₁₃ is CN, CF₃, OCF₃, ORₐ, or SRₐ. In some embodiments, R₁₃ is halogen, NRₐR_{b}, —C₁₋₄alkyl-SRₐ, or —C₁₋₄alkyl-ORₐ. In some embodiments, R₁₃ is ORₐ, SRₐ, or NRₐR_{b}. In some embodiments, R₁₃ is H, halogen, fluorinated alkyl, or alkyl. In some embodiments, R₁₃ is CH₃, CH₂CH₃, OH, F, Cl, Br, OCH₃, CH₂OCH₃, CF₃, CN, C≡CH, or

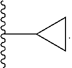 [SMILES: *C1CC1]

In some embodiments, R₁₃ is H, Me, Et, i-Pr, n-Bu, CF₂H, CF₂Cl, or CF₃. In some embodiments, R₁₃ is OH, OCH₃, CH₂OCH₃. In some embodiments, R₁₃ is halogen, e.g., Cl, F, Br, or I. In some embodiments, R₁₃ is Cl. In some embodiments, R₁₃ is halogenated alkyl, e.g., CF₃, CH₂F, CH₂Cl, CH₂CF₃, CHFCH₃, CHFCH₂F, CF₂CH₃, CHClCH₃, CCl₂CH₃, CHBrCH₃, CH₂CH₂CF₃, or CHClCHClCH₃. In some embodiments, R₁₃ is halogenated cycloalkyl, e.g.,

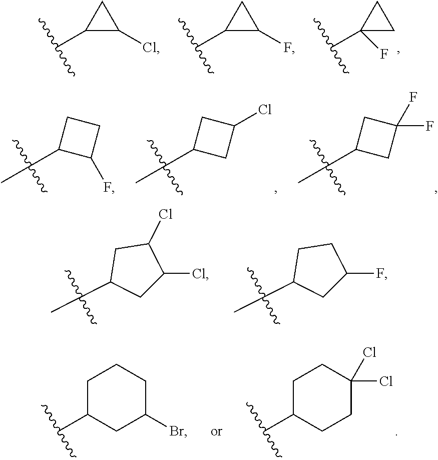

In some embodiments, R₁₃ is CN. In some embodiments, R₁₃ is alkyenyl, e.g., ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, or (E)-pent-1-enyl. In some embodiments, R₁₃ is ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl.

In some embodiments, the compound of Formula I, Ia, Ib, Ic, IIa, IIb, or IIc is any one of the compounds described herein. In some embodiments, the compound of Formula I, Ia, Ib, Ic, IIa, IIb, or IIc is selected from the group consisting of compounds in Examples 2-5 and Tables 1-5. In some embodiments, the compound of Formula I, Ia, Ib, Ic, IIa, IIb, or IIc is selected from the group consisting of compounds in Examples 2-5 and Tables 1, 2 and 3. In some embodiments, the compound of Formula I, Ia, Ib, Ic, IIa, IIb, or IIc is selected from the group consisting of compounds in Tables 4-5. In some embodiments, the compound of Formula I, Ia, Ib, IIa, IIc is selected from the group consisting of compounds in Examples 2-5 and Tables 1 and 3. In some embodiments, the compound of Formula I, Ia, Ib, Ic, IIa, IIb, or IIc is selected from the group consisting of compounds in Table 2. The enumerated compounds in Tables 1-5 are representative and non-limiting compounds of the embodiments disclosed herein.

### Abbreviations

ACN Acetonitrile
DCM Dichloromethane
DIEA N,N-Diisopropylethylamine
DMF Dimethyl formamide
DMSO Dimethyl sulfoxide
EA Ethyl acetate
MeOH Methanol
NMP N-Methyl-2-Pyrrolidone
PE Petroleum ether
TFA Trifluoroacetic acid
THF Tetrahydrofuran

### Methods of Preparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations, but not limitations of the preparation of some of the starting materials and compounds disclosed herein.

Schemes 1-8 below describe synthetic routes which may be used for the synthesis of compounds of the present invention, e.g., compounds having a structure of Formula I, Ia, Ib, Ic, IIa, IIb, or IIc, or a precursor thereof. Various modifications to these methods may be envisioned by those skilled in the art to achieve similar results to that of the inventions given below. In the embodiments below, the synthetic route is described using compounds having the structure of Formula I, Ia, Ib, Ic, IIa, IIb, or IIc, or a precursor thereof as examples. The general synthetic routes described in Schemes 1-8 and examples described in the Example section below illustrate methods used for the preparation of the compounds described herein.

Compound I-3 as shown in Scheme 1 can be prepared by any method known in the art and/or is commercially available. X refers to a leaving group. Non-limiting examples of the leaving groups include Cl, Br, or I. Other substituents are defined herein. As shown in Scheme 1, compounds of Formula I, such as I-1, can be prepared by alkylation of a suitably substituted pyridazinone I-3 with a halomethyl oxadiazole I-2 in the presence of a base such as potassium carbonate, optionally with a catalyst such as sodium iodide in a solvent such as DMF or NMP.

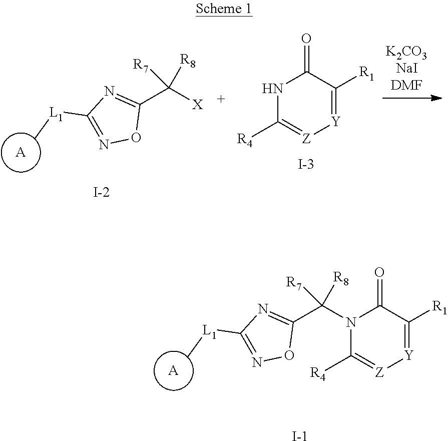

Compound I-4 as shown in Scheme 2 can be prepared by any method known in the art and/or is commercially available. Substituents shown in Scheme 2 are defined herein. As shown in Scheme 2, oxadiazole I-2 can be prepared from a nitrile I-4 as shown in Scheme 2. Nitrile I-4 is converted to the amide oxime I-5 by heating with hydroxylamine hydrochloride and a base such as sodium bicarbonate in a solvent such as ethanol. Alternatively, hydroxylamine solution in water can be used without an added base. The amide oxime is reacted with α-haloacyl halide such as chloroacetyl chloride and a base such as triethylamine. The resulting intermediate is cyclized to the chloromethyl oxadiazole in toluene by heating, for example at 100° C.

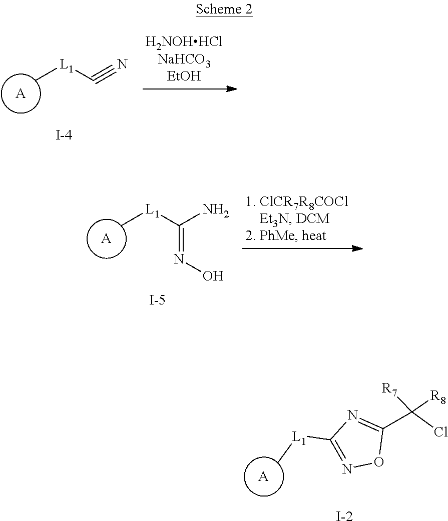

Compound I-3 as shown in Scheme 3 can be prepared by any method known in the art and/or is commercially available. Substituents shown in Scheme 3 are defined herein. As shown in Scheme 3, a second way to synthesize the compounds of Formula I, such as I-1, is to construct the oxadiazole ring from a pyridazine acetic acid and an amide oxime. A suitably substituted pyridazinone I-3 is reacted with a haloacetic ester such as ethyl bromoacetate in the presence of a base such as potassium carbonate to yield ester I-6. The ester is then hydrolyzed, for example with lithium hydroxide, to give carboxylic acid I-7. Acid I-7 and amide oxime I-5 are reacted together with a coupling agent such as propanephosphonic anhydride and a base such as diisopropylethylamine in a solvent such as DCM. The adduct formed is then heated in a solvent such as DMF to bring about cyclization to form oxadiazole I-1.

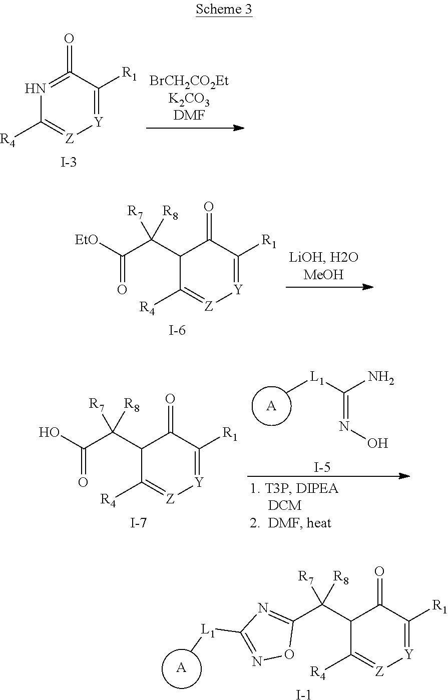

Many heterocycles I-3 are available where one or more of R₁, R₂, R₃ and R₄ are halogens. Those can be used to prepare alkyl, aryl cyano or other functionalized heterocycles by cross-coupling chemistry, e.g., Suzuki reaction. Such transformations may be carried out on the heterocycle before reacting with I-2 to give I-1, or a halogen substituted heterocycle I-3 can be reacted with I-2, and then the halogen converted to other substituents. Similarly, heterocycle carboxylic acids and esters I-3 can be used as precursors for amides, nitrile, alkoxymethyl, hydroxymethyl and aminomethyl substituents. The chemistry may be carried out either before or after coupling I-3 with I-2 to give I-1.

Compound I-8 as shown in Scheme 4 can be prepared by any method known in the art and/or is commercially available. Substituents shown in Scheme 4 are defined herein. Compounds of Formula I wherein L₁ is (S)—CH(OH)CH₂ can be obtained from ketonitrile I-8. Reduction of the ketone with a suitable chiral reducing agent gives the S-alcohol I-9. One such chiral reducing agent is [N-[(1S,2S)-2-(amino-κN)-1,2-diphenylethyl]-4-methylbenzenesulfonamidato-κN]chloro[(1,2,3,4,5,6-f)-1,3,5-trimethylbenzene]-ruthenium (CAS [174813-81-1]) in a mixture of formic acid and triethylamine. The alcohol I-9 is then converted to amide oxime I-5a and chloromethyl oxadiazole I-2a by the same methods used to prepare I-2 (as in Scheme 2).

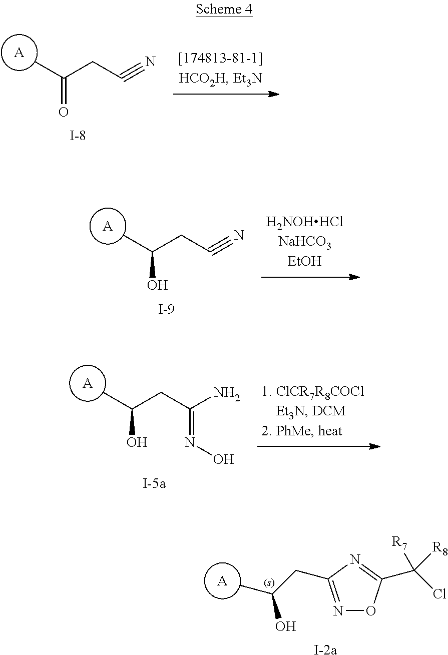

A heterocycle I-10 where both X groups are halogens is reacted with an aryl or vinyl boronic acid or boronate ester R₂B(OH)₂ in the presence of a palladium catalyst such as Pd(dppf)Cl₂ and a base such as sodium carbonate in a solvent such as dioxane and water to give I-11 where R₂ is aryl or vinyl as shown in Scheme 5. The NH is then protected with a suitable protecting group (PG) such as tetrahydropyranyl (THP) to form I-12. When PG is THP, it is introduced by treating I-11 with dihydropyran and an acid such as toluene sulfonic acid. The PG may also be added first by protecting I-10. I-12 is then reacted with R₁B(OH)₂, as the free boronic acid, boronate ester or boroxine and a palladium catalyst such as tetrakistriphenylphosphine palladium in the presence of a base such as potassium carbonate in a solvent such as dioxane and water. Deprotection of I-13 yields I-14 that can be converted to compound I-1 by either of the methods outlined in Scheme 1 or Scheme 3.

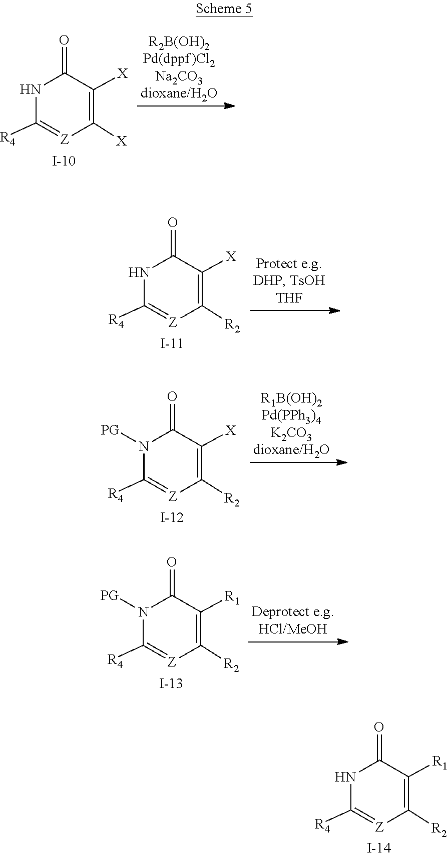

Compounds where Y is CR₂, Z is N and R₂ is an oxygenated substituent can be synthesized from pyrimidone ester I-15 as shown in Scheme 6.

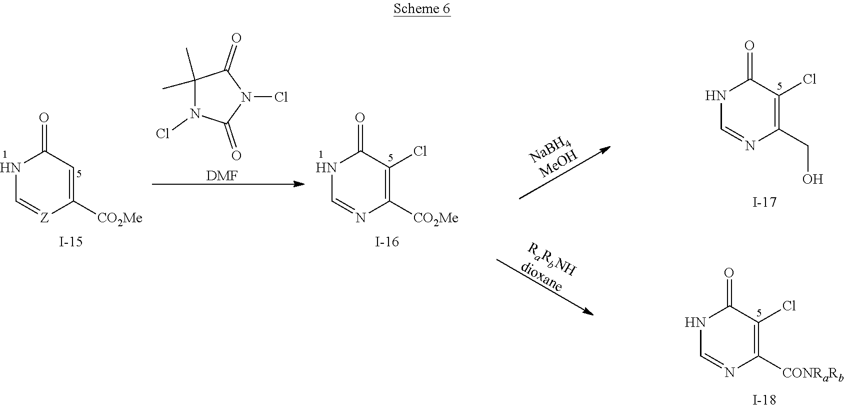

The R₁ group can be introduced by halogenating I-15 at C5 with for example 1,3-dichloro-5,5-dimethyl hydantoin in a solvent such as DMF. Reduction of the ester with a reducing agent such as sodium borohydride in methanol gives the hydroxymethyl pyrimidinone I-17. Treatment of I-16 with an amine RₐR_{b}NH in a solvent such as dioxane provides amide I-18. The chlorine atom at C5 in 1-16, 1-17 or I-18 can be converted into other R₁ groups by standard methods such as Suzuki reaction.

Compounds where Y is CR₂, Z is N and R₄ is an oxygenated substituent are obtained from 2,5-dichloro-4-methoxypyrimidine I-19 as shown in Scheme 7.

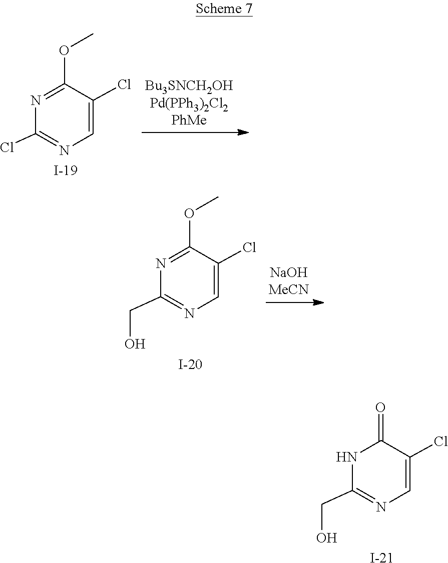

Stille reaction of I-19 with a hydroxymethyl stannane and a palladium^{II} catalyst such as bistriphenylphosphine palladium dichloride in a solvent such as toluene yields the hydroxymethyl pyrimidine I-20. Hydrolysis with a base such as sodium hydroxide gives pyrimidone I-21. The chlorine atom can be converted to other R₁ groups be standard methods. One way to prepare compounds where Y is CR₂ and Z is CH is from the pyridine boronic acid I-22 as shown in Scheme 8.

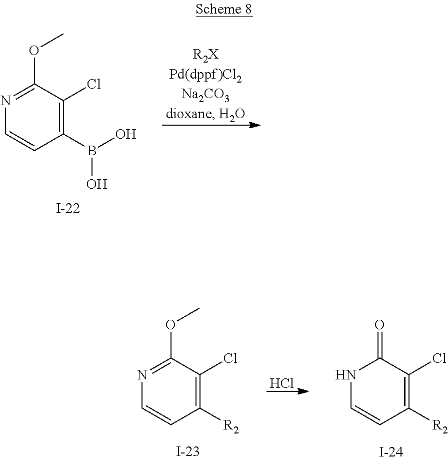

Reaction of boronic acid I-22 with an aryl (or heteroaryl) halide R₂X where X is Cl, Br or I, a palladium catalyst such as Pd(dppf)Cl₂, and a base such as sodium carbonate in a solvent such as dioxane and water gives I-23 where R₂ is aryl or heteroaryl. Hydrolysis with an acid such as HCl provides pyridine I-24.

### Pharmaceutical Compositions

This invention also provides a pharmaceutical composition comprising at least one of the compounds as described herein or a pharmaceutically acceptable salt or solvate thereof, and a pharmaceutically acceptable carrier or diluent.

In yet another aspect, the present invention provides a pharmaceutical composition comprising at least one compound selected from the group consisting of compounds of Formula I, Ia, Ib, Ic, IIa, IIb, or IIc as described herein and a pharmaceutically acceptable carrier or diluent.

In certain embodiments, the composition is in the form of a hydrate, solvate or pharmaceutically acceptable salt. The composition can be administered to the subject by any suitable route of administration, including, without limitation, oral and parenteral.

The phrase “pharmaceutically acceptable carrier” as used herein means a pharmaceutically acceptable material, composition or vehicle, such as a liquid or solid filler, diluent, excipient, solvent, or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as butylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

In certain embodiments, the compounds in the pharmaceutical compositions may be provided in the form of pharmaceutically acceptable salts. The term “pharmaceutically acceptable salt,” as used herein, refers to the relatively non-toxic, inorganic and organic acid salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. See, e.g., Berge et al., (1977) “Pharmaceutical Salts”, J. Pharm. Sci. 66:1-19 (incorporated herein by reference in its entirety).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like. See, e.g., Berge et al. (supra).

Wetting agents, emulsifiers, and lubricants, such as sodium lauryl sulfate, magnesium stearate, and polyethylene oxide-polybutylene oxide copolymer, as well as coloring agents, release agents, coating agents, sweetening, flavoring and perfuming agents, preservatives, and antioxidants can also be present in the compositions.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions, to provide the desired release profile, other polymer matrices, liposomes, and/or microspheres. They may be sterilized by, for example, filtration through a bacteria-retaining filter, or by incorporating sterilizing agents in the form of sterile solid compositions, which can be dissolved in sterile water or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar, and tragacanth, and mixtures thereof.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving or dispersing the pharmaceutical agents in the proper medium. Absorption enhancers can also be used to increase the flux of the pharmaceutical agents of the invention across the skin. The rate of such flux can be controlled, by either providing a rate-controlling membrane or dispersing the compound in a polymer matrix or gel.

Ophthalmic formulations, eye ointments, powders, solutions, and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions, or emulsions; or sterile powders which may be reconstituted into sterile injectable solutions or dispersions just prior to use, which may contain antioxidants, buffers, bacteriostats, or solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. One strategy for depot injections includes the use of polyethylene oxide-polypropylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot-injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), racehorses, birds, lizards, and any other organism which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

### Administration to a Subject/Methods of Treating a Condition

In yet another aspect, the present invention provides a method for treating a condition in a mammalian species in need thereof, the method comprising administering to the mammalian species a therapeutically effective amount of at least one compound selected from the group consisting of compounds of Formula I, Ia, Ib, Ic, IIa, IIb, or IIc, or a pharmaceutically acceptable salt thereof or a pharmaceutical composition containing any one of the compounds or pharmaceutically acceptable salts thereof, wherein the condition is selected from the group consisting of pain, a skin disorder, a respiratory disease, a fibrotic disease, an inner ear disorder, fever or another disorder of thermoregulation, a urinary tract or bladder disorder, an autoimmune disease, ischemia, a central nervous system (CNS) disorder, an inflammatory disorder, a gastroenterological disorder, and a cardiovascular disorder.

In some embodiments, the pain is acute pain, chronic pain, complex regional pain syndrome, inflammatory pain, neuropathic pain, postoperative pain, rheumatoid arthritic pain, osteoarthritic pain, back pain, visceral pain, cancer pain, algesia, neuralgia, migraine, neuropathies, diabetic neuropathy, sciatica, HIV-related neuropathy, pos-herpetic neuralgia, fibromyalgia, nerve injury, post stock pain, or tooth and tooth injury-related pain.

In some embodiments, the urinary tract or bladder disorder is pelvic hypersensitivity, urinary incontinence, cystitis, bladder instability, or bladder outlet obstruction. In some embodiments, the skin disorder is burns, psoriasis, eczema, or pruritus. In some embodiments, the skin disorder is atopic dermatitis or psoriasis-induced itching.

In some embodiments, the respiratory disease is an inflammatory airway disease, airway hyperresponsiveness, an idiopathic lung disease, chronic obstructive pulmonary disease, asthma, chronic asthma, tracheobronchial or diaphragmatic dysfunction, cough, or chronic cough.

In some embodiments, the ischemia is CNS hypoxia or a disorder associated with reduced blood flow to CNS. In some embodiments, the autoimmune disease is rheumatoid arthritis or multiple sclerosis. In some embodiments, the central nervous system disorder is associated with neurodegeneration. In some embodiments, the gastroenterological disorder is an inflammatory bowel disease, esophagitis, gastroesophageal reflux disorder, irritable bowel syndrome, emesis, or stomach duodenal ulcer. In some embodiments, the cardiovascular disorder is stroke, myocardial infarction, atherosclerosis, or cardiac hypertrophy.

In some embodiments, the mammalian species is human.

In yet another aspect, a method of inhibiting transient receptor potential ankyrin 1 (TRPA1) in a mammalian species in need thereof is described, including administering to the mammalian species a therapeutically effective amount of at least one compound of Formula I, Ia, Ib, Ic, IIa, IIb, or IIc, or a pharmaceutically acceptable salt thereof or pharmaceutical composition containing any one of the compounds or pharmaceutically acceptable salts thereof.

In some embodiments, the compounds described herein is selective in inhibiting TRPA1 with minimal or no off-target inhibition activities against potassium channels, or against calcium or sodium channels. In some embodiments, the compounds described herein do not block the hERG channels and therefore have desirable cardiovascular safety profiles.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants; even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops, or preparations with protracted release of active compounds in whose preparation excipients and additives and/or auxiliaries such as disintegrants, binders, coating agents, swelling agents, lubricants, flavorings, sweeteners, or solubilizers are customarily used as described above. The pharmaceutical compositions are suitable for use in a variety of drug delivery systems. For a brief review of present methods for drug delivery, see Langer, R. (1990) Science 249:1527-33, which is incorporated herein by reference in its entirety.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, different doses may be necessary depending on activity of the compound, manner of administration, purpose of the administration (i.e., prophylactic or therapeutic), nature and severity of the disorder, age and body weight of the patient. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically acceptable salt. When used in medicine the salts should be pharmaceutically acceptable, but non-pharmaceutically acceptable salts can conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium, or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, PA; incorporated herein by reference in its entirety.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained-release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids, or neutral fats such as mono-di- and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974, and 5,407,686. In addition, pump-based hardware delivery systems can be used, some of which are adapted for implantation.

### Assays for Effectiveness of TRPA1 Channel Inhibitors

In some embodiments, the compounds as described herein are tested for their activities against TRPA1 channel. In some embodiments, the compounds as described herein are tested for their TRPA1 channel electrophysiology. In some embodiments, the compounds as described herein are tested for their hERG electrophysiology.

### Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

Example 1 (including Example 1A-1F) describes exemplary intermediates used in the syntheses of representative compounds of Formula I, Ia, Ib, Ic, IIa, IIb, or IIc disclosed herein.

### Example 1A. Intermediate 1 ((1S)-2-[5-(chloromethyl)-1,2,4-oxadiazol-3-yl]-1-(4-chlorophenyl)ethanol)

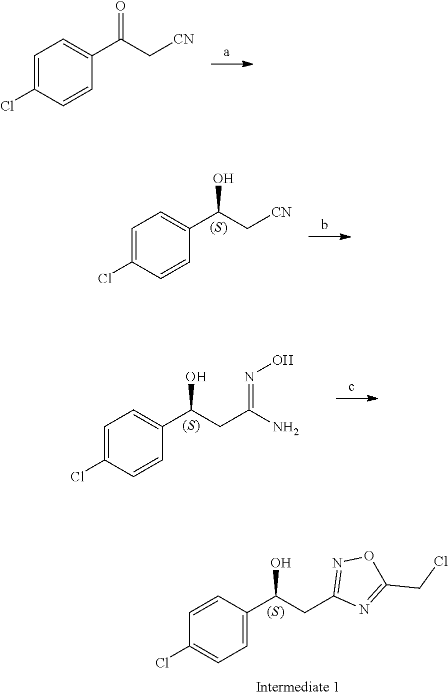

### Step a:

To a stirred solution of 3-(4-chlorophenyl)-3-oxopropanenitrile (30.0 g, 167 mmol) and RuCl[(S, S)-Tsdpen](mesitylene) (0.426 g, 0.680 mmol) in ACN (300 mL) was added formic acid triethylamine complex (5:2) (24 mL) at 0° C. under nitrogen atmosphere. The reaction mixture was stirred at room temperature for 3 h and concentrated under reduced pressure. The residue was dissolved in EA (200 mL) and water (300 mL) and extracted with EA (3×300 mL). The combined organic layers were washed with brine (2×300 mL) and dried over anhydrous Na₂SO₄. After filtration, the filtrate was concentrated under reduced pressure to afford (3S)-3-(4-chlorophenyl)-3-hydroxypropanenitrile as a brown oil (30.0 g, crude), which was used directly in the next step without purification: ¹H NMR (400 MHz, DMSO-d₆) δ 7.50-7.37 (m, 4H), 6.03 (d, J=4.6 Hz, 1H), 4.95-4.86 (m, 1H), 2.86 (m, 2H).

### Step b:

A solution of (3S)-3-(4-chlorophenyl)-3-hydroxypropanenitrile (30.0 g, 165 mmol) and NH₂OH (50% in water) (24 mL) in MeOH (300 mL) was stirred at 75° C. for 16 h. The mixture was cooled to room temperature and concentrated under reduced pressure to afford (3S)-3-(4-chlorophenyl)-N,3-dihydroxypropanimidamide as a brown oil (30.0 g, crude), which was used directly in the next step without purification: LCMS (ESI) calc'd for C₉H₁₁ClN₂O₂ [M+H]⁺: 215, 217 (3:1) found 215, 217 (3:1); ¹H NMR (400 MHz, DMSO-d₆) δ 8.76 (s, 1H), 7.38-7.33 (m, 4H), 5.53-5.35 (m, 3H), 4.95-4.79 (m, 1H), 2.39-2.14 (m, 2H).

### Step c:

To a stirred solution of (3S)-3-(4-chlorophenyl)-N,3-dihydroxypropanimidamide (30.0 g, 140 mmol) and DIEA (45.2 g, 349 mmol) in NMP (300 mL) was added chloroacetyl chloride (17.4 g, 154 mmol) at 0° C. The resulting mixture was stirred at 0° C. for 2 h, heated to 95° C., stirred for 4 h and cooled to room temperature. The mixture was diluted with EA (300 mL) and water (200 mL) and the layers separated. The aqueous layer was extracted with more EA (3×500 mL). The combined organic layers were washed with brine (3×500 mL) and dried over anhydrous Na₂SO₄. After filtration, the filtrate was concentrated under reduced pressure. The residue was purified by silica gel column chromatography, eluting with PE/EA (5/1) to afford (1S)-2-[5-(chloromethyl)-1,2,4-oxadiazol-3-yl]-1-(4-chlorophenyl)ethanol as a yellow solid (15.0 g, 33.0% over three steps); LCMS (ESI) calc'd for C₁₁H₁₀Cl₂N₂O₂ [M−H]⁻: 271, 273 (3:2) found 271, 273 (3:2); ¹H NMR (300 MHz, DMSO-d₆) δ 7.53-7.23 (m, 4H), 5.67 (d, J=4.9 Hz, 1H), 5.09 (s, 2H), 5.05-4.96 (m, 1H), 3.11-2.96 (m, 2H).

### Example 1B. Intermediate 2 (5-methyl-6-(1H-pyrazol-4-yl)-3H-pyrimidin-4-one)

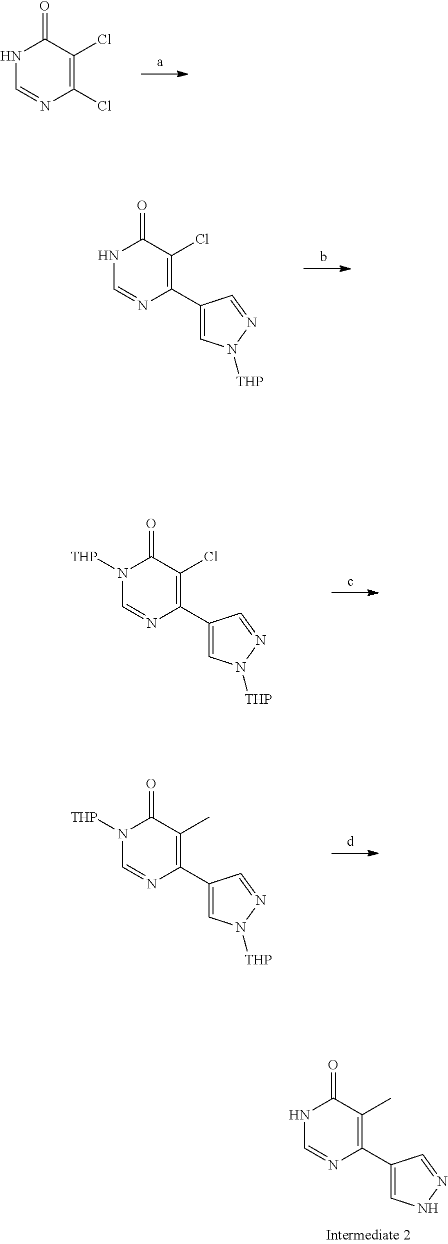

To a stirred solution of 5,6-dichloro-3,4-dihydropyrimidin-4-one (0.500 g, 3.03 mmol) and 1-(tetrahydropyran-2-yl)-4-(4,4,5,5-tetramethyl-1,3,2-dioxaborolan-2-yl)pyrazole (1.26 g, 4.55 mmol) in dioxane (8 mL) and H₂O (2 mL) were added Na₂CO₃ (0.964 g, 9.09 mmol) and Pd(dppf)Cl₂·CH₂Cl₂ (0.247 g, 0.303 mmol) at room temperature. The reaction was degassed under reduced pressure and purged with nitrogen three times and then stirred at 80° C. for 12 h. After cooling to room temperature, the resulting mixture was filtered and the filtered cake was washed with MeOH (3×3 mL). The filtrate was concentrated under reduced pressure. The residue was purified by silica gel chromatography, eluting with PE/EA (5/1) to afford 5-chloro-6-[1-(tetrahydropyran-2-yl)pyrazol-4-yl]-3H-pyrimidin-4-one as an off-white solid (0.450 g, 52.9%): LCMS (ESI) calc'd for C₁₂H₁₃ClN₄O₂ [M+H]⁺: 281, 283 (3:1) found 281, 283 (3:1); ¹H NMR (300 MHz, DMSO-d₆) δ 8.63 (d, J=0.7 Hz, 1H), 8.40-8.21 (m, 1H), 8.19 (d, J=3.6 Hz, 1H), 5.54 (t, J=9.8 Hz, 1H), 4.06-3.84 (m, 1H), 3.75-3.57 (m, 1H), 2.22-2.04 (m, 1H), 2.04-1.84 (m, 2H), 1.79-1.48 (m, 3H).

### Step b:

To a stirred mixture of 5-chloro-6-[1-(tetrahydropyran-2-yl)pyrazol-4-yl]-3H-pyrimidin-4-one (0.450 g, 1.60 mmol) and DHP (0.337 g, 4.01 mmol) in THE (5 mL) was added TsOH·H₂O (76.2 mg, 0.401 mmol) at room temperature under nitrogen atmosphere. The resulting mixture was stirred at 70° C. for 16 h under nitrogen atmosphere. After cooling to room temperature, the resulting mixture was quenched with water (30 mL) and extracted with EA (3×50 mL). The combined organic layers were washed with brine (2×30 mL) and dried over anhydrous Na₂SO₄. After filtration, the filtrate was concentrated under reduced pressure. The residue was purified by silica gel chromatography, eluting with PE/EA (1/1) to afford 5-chloro-3-(tetrahydropyran-2-yl)-6-[1-(tetrahydropyran-2-yl)pyrazol-4-yl]pyrimidin-4-one as a light yellow oil (0.262 g, 44.8%): LCMS (ESI) calc'd for C₁₇H₂₁ClN₄O₃ [M+H]+: 365, 367 (3:1) found 365, 367 (3:1); ¹H NMR (300 MHz, CDCl₃) δ 8.97-7.77 (m, 3H), 5.90-5.32 (m, 2H), 4.43-4.06 (m, 2H), 3.84-3.62 (m, 2H), 2.18-1.94 (m, 4H), 1.78-1.50 (m, 8H).

### Step c:

To a solution of 5-chloro-3-(tetrahydropyran-2-yl)-6-[1-(tetrahydropyran-2-yl)pyrazol-4-yl]pyrimidin-4-one (0.160 g, 0.439 mmol) and trimethyl boroxine (0.275 g, 2.19 mmol) in dioxane (1.6 mL) and H₂O (0.4 mL) were added Cs₂CO₃ (0.429 g, 1.32 mmol) and Pd(PPh₃)₄ (50.7 mg, 0.0440 mmol) at room temperature. The reaction was degassed under reduced pressure and purged with nitrogen three times and then stirred at 100° C. for 4 h. After cooling to room temperature, the resulting mixture was filtered, and the filtered cake was washed with MeOH (3×10 mL). The filtrate was concentrated under reduced pressure. The residue was purified by reverse phase chromatography, eluting with 35% ACN in water (plus 10 mmol/L NH₄HCO₃) to afford 5-methyl-3-(tetrahydropyran-2-yl)-6-[1-(tetrahydropyran-2-yl)pyrazol-4-yl]pyrimidin-4-one as a yellow oil (74.0 mg, 48.9%): LCMS (ESI) calc'd for C₁₈H₂₄N₄O₃ [M+H]⁺: 345 found 345; ¹H NMR (300 MHz, DMSO-d₆) δ 8.35 (d, J=5.7 Hz, 2H), 8.00 (s, 1H), 5.83-5.65 (m, 1H), 5.50 (t, J=10.0 Hz, 1H), 4.15-3.89 (m, 2H), 3.69-3.61 (m, 2H), 2.18 (s, 3H), 2.03-1.44 (m, 12H).

### Step d:

A solution of 5-methyl-3-(tetrahydropyran-2-yl)-6-[1-(tetrahydropyran-2-yl)pyrazol-4-yl]pyrimidin-4-one (74.0 mg, 0.215 mmol) and aq. HCl (0.2 mL, 6 M) in MeOH (0.8 mL) was stirred at room temperature for 2 h. The resulting mixture was concentrated under reduced pressure to afford 5-methyl-6-(1H-pyrazol-4-yl)-3H-pyrimidin-4-one as a yellow oil (46.0 mg, crude), which was used in the next step directly without further purification: LCMS (ESI) calc'd for C₈H₈N₄O [M+H]⁺: 177 found 177.

### Example 1C. Intermediate 3 (5-chloro-6-(hydroxymethyl)pyrimidin-4(3H)-one)

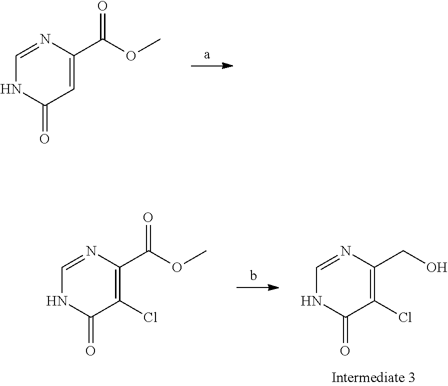

### Step a:

To a stirred mixture of methyl 6-oxo-1,6-dihydropyrimidine-4-carboxylate (1.00 g, 6.49 mmol) in DMF (15 mL) was added 1,3-dichloro-5,5-dimethylimidazolidine-2,4-dione (0.770 g, 3.89 mmol) at room temperature. The resulting mixture was stirred at room temperature for 12 h. The resulting mixture was purified by reverse phase chromatography, eluting with 20% ACN in water (plus 0.1% TFA) to afford methyl 5-chloro-6-oxo-1,6-dihydropyrimidine-4-carboxylate as a light yellow solid (0.700 g, 57.2%): LCMS (ESI) calc'd for C₆H₅ClN₂O₃ [M+H]⁺: 189, 191 (3:1) found 189, 191 (3:1); ¹H NMR (300 MHz, DMSO-d₆) δ 13.78 (s, 1H), 8.31 (s, 1H), 3.90 (s, 3H).

### Step b:

To a stirred mixture of methyl 5-chloro-6-oxo-1,6-dihydropyrimidine-4-carboxylate (0.300 g, 1.59 mmol) in THE (4 mL) and MeOH (1 mL) was added NaBH₄ (0.241 g, 6.36 mmol) at room temperature. The reaction was stirred at 70° C. for 4 h under nitrogen atmosphere. The resulting mixture was concentrated under reduced pressure. The residue was purified by reverse phase chromatography, eluting with 1% ACN in water (plus 0.1% TFA) to afford 5-chloro-6-(hydroxymethyl)pyrimidin-4(3H)-one as a light yellow solid (0.300 g, crude), which was used to next step directly without further purification: LCMS (ESI) calc'd for C₅H₅ClN₂O₂ [M+H]⁺: 161, 163 (3:1) found 161, 163 (3:1).

### Example 1D. Intermediate 4 (5-chloro-6-oxo-1H-pyrimidine-4-carboxamide)

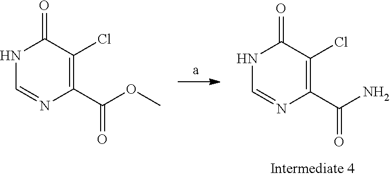

### Step a:

To a stirred solution of methyl 5-chloro-6-oxo-1H-pyrimidine-4-carboxylate (0.600 g, 3.18 mmol) in dioxane (1 mL) was added NH₃·H₂O (5 mL, 25%) at room temperature. The reaction was stirred at room temperature for 16 h. The resulting solution was concentrated under reduced pressure. The residue was purified by reverse phase chromatography, eluting with 3% ACN in water (plus 0.05% TFA) to afford 5-chloro-6-oxo-1H-pyrimidine-4-carboxamide as an off-white solid (0.240 g, 43.5%): LCMS (ESI) calc'd for C₅H₄ClN₃O₂ [M+H]⁺: 174, 176 (3:1) found 174, 176 (3:1); ¹H NMR (300 MHz, DMSO-d₆) δ 10.54 (s, 1H), 8.24 (s, 1H), 8.01 (s, 1H), 7.79 (s, 1H).

### Example 1E. Intermediate 5 (5-chloro-2-(hydroxymethyl)-3H-pyrimidin-4-one)

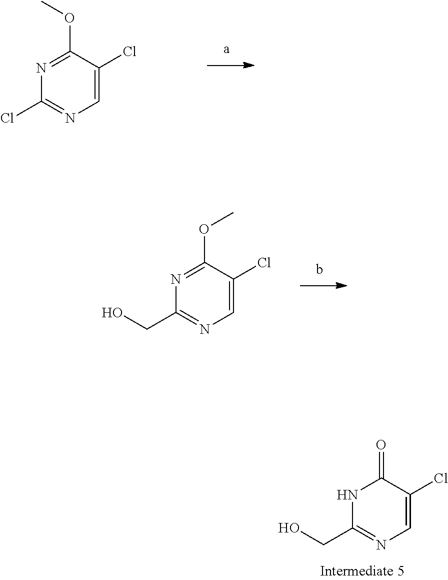

### Step a:

To a stirred solution of 2,5-dichloro-4-methoxypyrimidine (1.00 g, 5.59 mmol) and (tributylstannyl)methanol (2.69 g, 8.38 mmol) in toluene (10 mL) was added Pd(PPh₃)₂Cl₂ (0.390 g, 0.560 mmol) at room temperature. The reaction was degassed under reduced pressure and purged with nitrogen three times and then stirred at 80° C. for 2 h. After cooling to room temperature, the reaction mixture was concentrated under reduced pressure. The residue was purified by reverse phase chromatography, eluting with 20% ACN in water (plus 10 mmol/L NH₄HCO₃) to afford (5-chloro-4-methoxypyrimidin-2-yl) methanol as an off-white solid (0.340 g, 34.9%); LCMS (ESI) calc'd for C₆H₇ClN₂O₂ [M+H]+: 175, 177 (3:1) found 175, 177 (3:1); ¹H NMR (400 MHz, DMSO-d₆) δ 8.64 (s, 1H), 4.90-4.87 (brs, 1H), 4.52 (s, 2H), 4.04 (s, 3H).

### Step b:

To a stirred solution of (5-chloro-4-methoxypyrimidin-2-yl) methanol (0.150 g, 0.860 mmol) in ACN (1 mL) was added NaOH (0.100 g, 2.58 mmol) in H₂O (2 mL) at room temperature. The reaction was stirred at room temperature for 2 h under nitrogen atmosphere. The resulting mixture was neutralized with aq. HCl (1 M) to pH 7 followed by concentrated under reduced pressure to afford 5-chloro-2-(hydroxymethyl)-3H-pyrimidin-4-one (0.300 g, crude), which was used in the next step directly without further purification: LCMS (ESI) calc'd for C₅H₅ClN₂O₂ [M−H]⁻: 159,161 (3:1) found 159,161 (3:1); ¹H NMR (300 MHz, DMSO-d₆) δ 8.14 (s, 1H), 5.85 (s, 1H), 4.33 (s, 2H).

### Example 1F. Intermediate 6 (3-chloro-4-(1-{[2-(trimethylsilyl)ethoxy]methyl}pyrazol-4-yl)pyridin-2-ol)

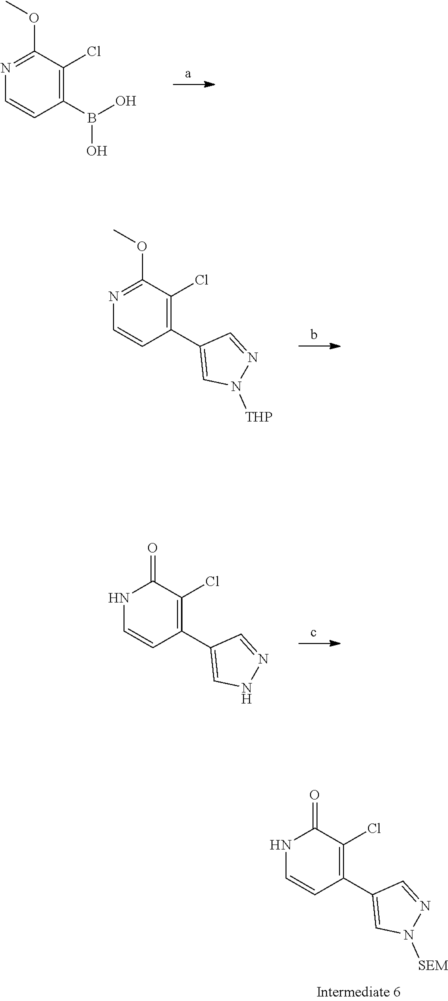

### Step a:

To a stirred mixture of 3-chloro-2-methoxypyridin-4-ylboronic acid (0.800 g, 4.27 mmol) and Na₂CO₃ (1.36 g, 12.8 mmol) in dioxane (8 mL) and H₂O (2 mL) were added 4-bromo-1-(tetrahydropyran-2-yl)pyrazole (0.990 g, 4.28 mmol) and Pd(dppf)Cl₂ (0.312 g, 0.427 mmol) at room temperature. The reaction was degassed under reduced pressure, purged with nitrogen three times and stirred at 80° C. for 16 h. The cooled mixture was diluted with water (50 mL) and extracted with EA (3×50 mL). The combined organic layers were washed with brine (3×50 mL), dried over anhydrous Na₂SO₄, filtered and concentrated under reduced pressure. The residue was purified by silica gel chromatography, eluting with PE/EA (2/1) to afford 3-chloro-2-methoxy-4-[1-(tetrahydropyran-2-yl)pyrazol-4-yl]pyridine as a light yellow oil (0.900 g, 71.8%): LCMS (ESI) calc'd for C₁₄H₁₆ClN₃O₂ [M+H]⁺: 294, 296 (3:1) found 294, 296 (3:1); ¹H NMR (300 MHz, DMSO-d₆) δ 8.59 (d, J=5.75 Hz, 1H), 8.15 (d, J=5.85 Hz, 1H), 8.11-8.03 (m, 1H), 7.36-7.28 (m, 1H), 5.56-5.47 (m, 1H), 4.05-3.90 (m, 4H), 3.74-3.61 (m, 1H), 2.22-2.06 (m, 1H), 2.03-1.89 (m, 2H), 1.81-1.44 (m, 3H).

### Step b:

A solution of 3-chloro-2-methoxy-4-[1-(tetrahydropyran-2-yl)pyrazol-4-yl]pyridine (0.400 g, 1.36 mmol) in aq. HCl (3 M, 4 mL) was stirred at 80° C. for 16 h. After cooling to room temperature, the resulting mixture was concentrated under reduced pressure to afford 3-chloro-4-(1H-pyrazol-4-yl)pyridin-2-ol as a colorless oil (0.230 g, crude), which was used in the next step directly without purification: LCMS (ESI) calc'd for C₈H₆ClN₃O [M+H]⁺: 196, 198 (3:1) found 196, 198 (3:1).

### Step c:

To a stirred mixture of 3-chloro-4-(1H-pyrazol-4-yl)pyridin-2-ol (0.230 g, 1.18 mmol) and K₂CO₃ (0.488 g, 3.53 mmol) in DMF (2.5 mL) was added SEMCl (0.196 g, 1.18 mmol) dropwise at room temperature. The reaction was stirred for 4 h, diluted with water (30 mL) and extracted with EA (3×30 mL). The combined organic layers were washed with brine (3×30 mL), dried over anhydrous Na₂SO₄, filtered and concentrated under reduced pressure. The residue was purified by silica gel chromatography, eluting with PE/EA (1/4) to afford 3-chloro-4-(1-{[2-(trimethylsilyl)ethoxy]methyl}pyrazol-4-yl)pyridin-2-ol as an off-white solid (0.160 g, 41.8%): LCMS (ESI) calc'd for C₁₄H₂₀ClN₃O₂Si [M+H]⁺: 326, 328 (3:1) found 326, 328 (3:1); ¹H NMR (300 MHz, DMSO-d₆) δ 11.99 (s, 1H), 8.70 (s, 1H), 8.20 (s, 1H), 7.42 (d, J=6.91 Hz, 1H), 6.58 (d, J=6.93 Hz, 1H), 5.53 (s, 2H), 3.68-3.61 (m, 2H), 0.90-0.84 (m, 2H), 0.00 (s, 9H).

Examples 2-5 describe the syntheses of representative compounds of Formula I, Ia, Ib, Ic, IIa, IIb, or IIc disclosed herein.

### Example 2. Compound 4 (1-({3-[2-(4-chlorophenyl)ethyl]-1,2,4-oxadiazol-5-yl}methyl)-5-methyl-6-oxopyridine-3-carboxamide)

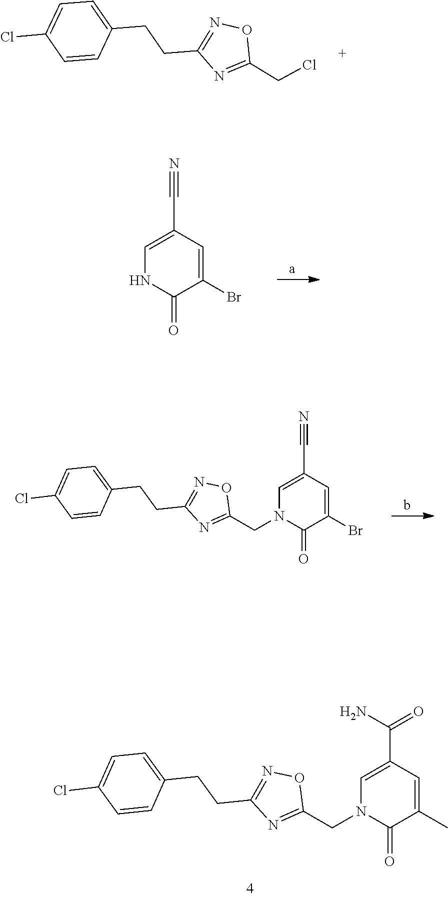 [SMILES: ClCc1nc(CCc2ccc(Cl)cc2)no1 ; N#Cc1c[nH]c(=O)c(Br)c1 ; N#Cc1cc(Br)c(=O)n(Cc2nc(CCc3ccc(Cl)cc3)no2)c1 ; Cc1cc(C(N)=O)cn(Cc2nc(CCc3ccc(Cl)cc3)no2)c1=O]

### Step a:

To a stirred solution of 5-(chloromethyl)-3-[2-(4-chlorophenyl)ethyl]-1,2,4-oxadiazole (0.200 g, 0.778 mmol) and 5-bromo-6-oxo-1H-pyridine-3-carbonitrile (0.232 g, 1.17 mmol) in DMF (2 mL) was added K₂CO₃ (0.215 g, 1.56 mmol) at room temperature. The reaction mixture was stirred for 2 h, diluted with water (10 mL) and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 63% ACN in water (plus 10 mM NH₄HCO₃) to afford 5-bromo-1-({3-[2-(4-chlorophenyl)ethyl]-1,2,4-oxadiazol-5-yl}methyl)-6-oxopyridine-3-carbonitrile as a brown solid (0.280 g, 85.8%): LCMS (ESI) calc'd for C₁₇H₁₂BrClN₄O₂ [M+H]⁺: 419, 421, 423 (2:3:1), found 419, 421, 423 (2:3:1); ¹H NMR (300 MHz, DMSO-d₆) δ 8.83 (d, J=2.3 Hz, 1H), 8.43 (d, J=2.2 Hz, 1H), 7.35-7.26 (m, 2H), 7.26-7.18 (m, 2H), 5.50 (s, 2H), 3.06-2.87 (m, 4H).

### Step b:

To a stirred mixture of 5-bromo-1-({3-[2-(4-chlorophenyl)ethyl]-1,2,4-oxadiazol-5-yl}methyl)-6-oxopyridine-3-carbonitrile (0.100 g, 0.238 mmol) and NaHCO₃ (40.0 mg, 0.476 mmol) in 1,4-dioxane (1 mL) and H₂O (0.5 mL) were added 2,4,6-trimethylboroxin (0.179 g, 1.43 mmol) and Pd(dppf)Cl₂·CH₂Cl₂ (17.4 mg, 0.0240 mmol) at room temperature under nitrogen atmosphere. The reaction mixture was degassed under vacuum and purged with nitrogen three times and then heated to 80° C. for 16 h. The resulting mixture was cooled to room temperature, diluted with water (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by Prep-HPLC with the following conditions: Column: SunFire Prep C18 OBD Column, 19×150 mm, 5 m; Mobile Phase A: Water (plus 0.05% TFA), Mobile Phase B: ACN; Flow rate: 20 mL/min; Gradient: 35% B to 60% B in 5 min, 60% B; Wavelength: UV 210 nm; Retention time: 4.98 min. The fractions containing the desired product were collected and concentrated under reduced pressure to afford 1-({3-[2-(4-chlorophenyl)ethyl]-1,2,4-oxadiazol-5-yl}methyl)-5-methyl-6-oxopyridine-3-carboxamide as a yellow solid (2.70 mg, 3.04%): LCMS (ESI) calc'd for C₁₈H₁₇ClN₄O₃ [M+H]⁺: 373, 375 (3:1) found 373, 375 (3:1); ¹H NMR (300 MHz, CD₃OD) δ 8.33 (d, J=2.53 Hz, 1H), 7.92-7.87 (m, 1H), 7.27-7.19 (m, 2H), 7.18-7.08 (m, 2H), 5.45 (s, 2H), 3.02-2.98 (m, 4H), 2.14 (s, 3H).

### Example 3. Compound 29 (4-({3-[(2S)-2-(4-chlorophenyl)-2-hydroxyethyl]-1,2,4-oxadiazol-5-yl}methyl)-6-methyl-5-oxopyrazine-2-carboxamide)

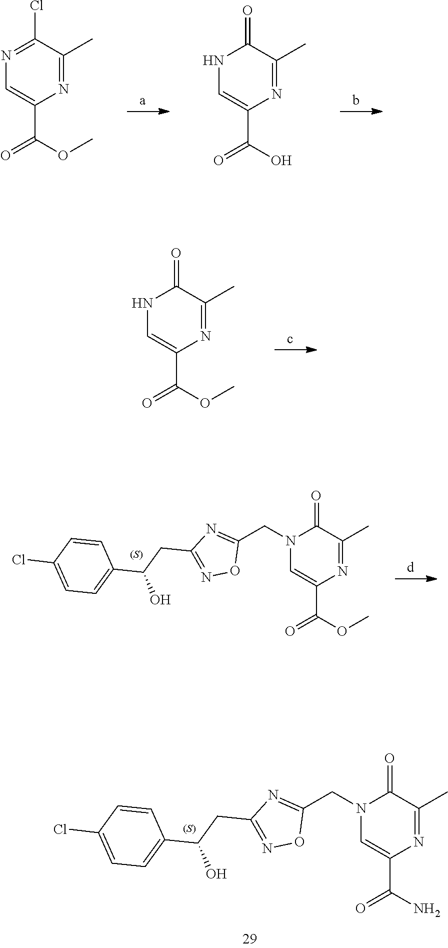

### Step a:

To a stirred mixture of methyl 5-chloro-6-methylpyrazine-2-carboxylate (2.00 g, 10.7 mmol) and LiOH (1.28 g, 53.6 mmol) in THE (14 mL) was added H₂O (7 mL) dropwise at room temperature. The reaction mixture was stirred for 16 h, concentrated under reduced pressure and acidified to pH 5 with HCl (1 M). The precipitate was collected by filtration and washed with water (4×5 mL) to afford 6-methyl-5-oxo-4,5-dihydropyrazine-2-carboxylic acid as an off-white solid (1.50 g, 90.8%): LCMS (ESI) calc'd for C₆H₆N₂O₃ [M+H]⁺: 155 found 155; ¹H NMR (300 MHz, DMSO-d₆) δ 12.62-12.58 (brs, 2H), 7.93 (s, 1H), 2.28 (s, 3H).

### Step b:

To a stirred mixture of 5-hydroxy-6-methylpyrazine-2-carboxylic acid (0.500 g, 3.24 mmol) and oxalyl chloride (2.05 g, 16.2 mmol) in DCM (15 mL) was added DMF (0.1 mL) dropwise at room temperature. The reaction mixture was stirred for 2 h and concentrated under reduced pressure. The residue was dissolved in MeOH (2 mL), stirred for 0.5 h and concentrated under reduced pressure. The residue was purified by reverse phase chromatography, eluting with 35% ACN in water (plus 0.05% TFA) to afford methyl 6-methyl-5-oxo-4,5-dihydropyrazine-2-carboxylateas a yellow solid (0.100 g, 18.3%): LCMS (ESI) calc'd for C₇H₈N₂O₃ [M+H]⁺: 169 found 169; ¹H NMR (300 MHz, DMSO-d₆) δ 12.60 (s, 1H), 7.99 (s, 1H), 3.78 (s, 3H), 2.28 (s, 3H).

### Step c:

To a stirred solution of methyl 5-hydroxy-6-methylpyrazine-2-carboxylate (80.0 mg, 0.470 mmol) and (1S)-2-[5-(chloromethyl)-1,2,4-oxadiazol-3-yl]-1-(4-chlorophenyl)ethanol (0.150 g, 0.570 mmol) in DMF (3 mL) were added K₂CO₃ (0.130 g, 0.950 mmol) and NaI (7.13 mg, 0.0500 mmol) at room temperature. The reaction mixture was stirred for 2 h, diluted with water (10 mL) and extracted with EA (4×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 50% ACN in water (plus 10 mM NH₄HCO₃) to afford methyl 4-({3-[(2S)-2-(4-chlorophenyl)-2-hydroxyethyl]-1,2,4-oxadiazol-5-yl}methyl)-6-methyl-5-oxopyrazine-2-carboxylate as a yellow solid (0.150 g, 77.9%): LCMS (ESI) calc'd for C₁₈H₁₇ClN₄O₅ [M+H]⁺: 405, 407 (3:1) found 405, 407 (3:1); ¹H NMR (300 MHz, CDCl₃) δ 8.14 (s, 1H), 7.35-7.31 (m, 4H), 5.32 (s, 2H), 5.16-5.12 (m, 1H), 3.96 (s, 3H), 3.17-3.10 (m, 2H), 2.56 (s, 3H).

### Step d:

Methyl 4-({3-[(2S)-2-(4-chlorophenyl)-2-hydroxyethyl]-1,2,4-oxadiazol-5-yl}methyl)-6-methyl-5-oxopyrazine-2-carboxylate (50.0 mg, 0.120 mmol) was added to NH₃ in MeOH (7 M, 1.5 mL), the reaction mixture was stirred at 40° C. for 4 h and concentrated under reduced pressure. The crude product (50.0 mg) was purified by Prep-HPLC with the following conditions Column: SunFire Prep C18 OBD Column, 19×150 mm, 5 m; Mobile Phase A: water (plus 0.05% TFA), Mobile Phase B: ACN; Flow rate: 25 mL/min; Gradient: 40% B to 50% B in 6.5 min, 50% B; Wavelength: UV 254/210 nm; Retention Time: 5.68 min. The fractions containing the desired product were collected and concentrated under reduced pressure to afford 4-({3-[(2S)-2-(4-chlorophenyl)-2-hydroxyethyl]-1,2,4-oxadiazol-5-yl}methyl)-6-methyl-5-oxopyrazine-2-carboxamide as an off-white solid (29.0 mg, 60.2%). LCMS (ESI) calc'd for C₁₇H₁₆ClN₅O₄ [M+H]⁺: 390, 392 (3:1) found 390, 392 (3:1); ¹H NMR (300 MHz, DMSO-d₆) δ 8.45 (s, 1H), 7.75 (s, 1H), 7.56 (s, 1H), 7.37-7.32 (m, 4H), 5.56-5.51 (m, 3H), 4.95-4.92 (m, 1H), 3.05-2.90 (m, 2H), 2.37 (s, 3H).

### Example 4. Compound 33 ((S)-6-amino-3-((3-(2-(4-chlorophenyl)-2-hydroxyethyl)-1,2,4-oxadiazol-5-yl)methyl)-5-methylpyrimidin-4(3H)-one)

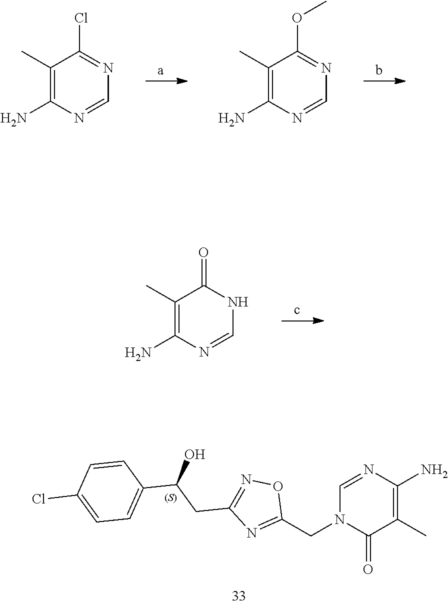

### Step a:

To a stirred mixture of 6-chloro-5-methylpyrimidin-4-amine (0.500 g, 3.48 mmol) in MeOH (8 mL) was added NaOMe (0.282 g, 5.22 mmol) at room temperature. The reaction mixture was stirred at 90° C. for 24 h, cooled to room temperature and concentrated under reduced pressure to afford 6-methoxy-5-methylpyrimidin-4-amine as an off-white solid (0.500 g, crude), which was used directly in the next step without purification: LCMS (ESI) calc'd for C₆H₉N₃O [M+H]⁺: 140, found 140.

### Step b:

A solution of 6-methoxy-5-methylpyrimidin-4-amine (0.500 g, 3.59 mmol) in HBr (5 mL, 33% in AcOH) was stirred at 100° C. for 1 h, cooled to room temperature and concentrated under reduced pressure. The residue was purified by silica gel column chromatography, eluting with CH₂Cl₂/MeOH (1/1) to afford 6-amino-5-methyl-3H-pyrimidin-4-one as a yellow solid (1.10 g, crude with silica), which was used directly in the next step without purification: LCMS (ESI) calc'd for C₅H₇N₃O [M+H]⁺: 126, found 126.

### Step c:

To a stirred mixture of (1S)-2-[5-(chloromethyl)-1,2,4-oxadiazol-3-yl]-1-(4-chlorophenyl)ethanol (0.100 g, 0.366 mmol) and 6-amino-5-methyl-3H-pyrimidin-4-one (0.206 g, 1.65 mmol) in DMF (1 mL) was added K₂CO₃ (0.101 g, 0.732 mmol) at room temperature. The reaction mixture was stirred for 1 h, diluted with water (20 mL) and extracted with EA (3×20 mL). The combined organic layers were washed with brine (5×2 mL) and dried over anhydrous Na₂SO₄. After filtration, the filtrate was concentrated under reduced pressure. The crude product was purified by Prep-HPLC with the following conditions: Column: X Bridge Prep OBD C18 Column, 19×250 mm, 5 m; Mobile Phase A: Water (plus 10 mM NH₄HCO₃), Mobile Phase B: ACN; Flow rate: 25 mL/min; Gradient: 28% B to 43% B in 6 min, 43% B; Detector: UV 254/220 nm; Retention time: 5.41 min. The fractions containing the desired product were collected and concentrated under reduced pressure to afford (S)-6-amino-3-((3-(2-(4-chlorophenyl)-2-hydroxyethyl)-1,2,4-oxadiazol-5-yl)methyl)-5-methylpyrimidin-4(3H)-one as an off-white solid (32.1 mg, 23.70%): LCMS (ESI) calc'd for C₁₆H₁₆ClN₅O₃ [M+H]⁺: 362, 364 (3:1) found 362, 364 (3:1); ¹H NMR (400 MHz, DMSO-d₆) δ 8.18 (s, 1H), 7.41-7.32 (m, 4H), 6.40 (s, 2H), 5.63 (d, J=4.9 Hz, 1H), 5.26 (s, 2H), 5.02-4.92 (m, 1H), 3.04-2.89 (m, 2H), 1.73 (s, 3H).

### Example 5. Compound 63 (5-chloro-1-({3-[(2S)-2-(4-chlorophenyl)-2-hydroxyethyl]-1,2,4-oxadiazol-5-yl}methyl)-6-oxopyrimidine-4-carbonitrile)

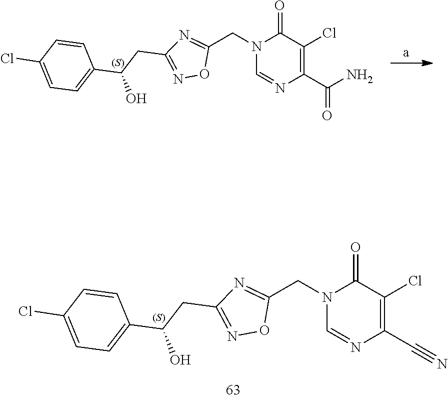

### Step a:

To a stirred solution of 5-chloro-1-({3-[(2S)-2-(4-chlorophenyl)-2-hydroxyethyl]-1,2,4-oxadiazol-5-yl}methyl)-6-oxopyrimidine-4-carboxamide (70.0 mg, 0.171 mmol) in pyridine (2 mL) was added TFAA (1 mL) dropwise at 0° C. The reaction was stirred at 25° C. for 2 h under nitrogen and concentrated under reduced pressure. The residue was purified by reverse phase chromatography, eluting with 50% ACN in water (plus 10 mM NH₄HCO₃) to afford 5-chloro-1-({3-[(2S)-2-(4-chlorophenyl)-2-hydroxyethyl]-1,2,4-oxadiazol-5-yl}methyl)-6-oxopyrimidine-4-carbonitrile as a brown solid (26.0 mg, 38.9%); LCMS (ESI) calc'd for C₁₆H₁₁C₁₂N₅O₃ [M−H]⁻: 390, 392 (3:2), found 390, 392 (3:2); ¹H NMR (300 MHz, DMSO-d₆) δ 8.81 (s, 1H), 7.35-7.31 (m, 4H), 5.63 (d, J=4.8 Hz, 1H), 5.55 (s, 2H), 4.98-4.92 (m, 1H), 3.05-2.94 (m, 2H).

The compounds in Table 1 below were prepared by using the procedures described in Schemes 1-8 or in an analogous fashion to that described for Compound 4, 29, 33, or 63 in Examples 2-5.

TABLE 1

| Compound | | | |
|---|---|---|---|
| No. | Structure | Chemical Name | MS: (M + H)⁺ & ¹H MNR |
| 1 | 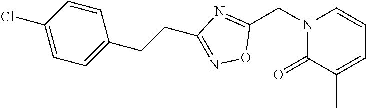 [SMILES: Cc1cccn(Cc2nc(CCc3ccc(Cl)cc3)no2)c1=O] | 1-((3-(4- chlorophenethyl)- 1,2,4-oxadiazol-5- yl)methyl)-3- methylpyridin- 2(1H)-one | [M + H]⁺: 330, 332 (3:1); ¹H NMR (300 MHz, CDCl₃) δ 7.69-7.58 (m, 1H), 7.53-7.45 (m, 1H), 7.29-7.23 (m, 2H), 7.23- 7.12 (m, 2H), 6.40-6.37 (m, 1H), 5.42 (s, 2H), 3.07- |
| | | | 2.95 (m, 4H), 2.13 (s, 3H). |
| 2 | 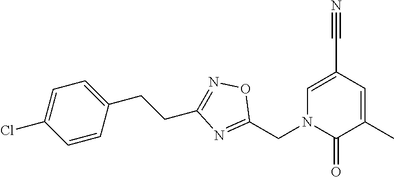 [SMILES: Cc1cc(C#N)cn(Cc2nc(CCc3ccc(Cl)cc3)no2)c1=O] | 1-((3-(4- chlorophenethyl)- 1,2,4-oxadiazol-5- yl)methyl)-5- methyl-6-oxo-1,6- dihydropyridine-3- carbonitrile | [M + H]⁺: 355, 357 (3:1); ¹H NMR (300 MHz, DMSO-d₆) δ 8.64-8.62 (m, 1H), 7.71-7.67 (m, 1H), 7.33-7.27 (m, 2H), 7.26- 7.21 (m, 2H), 5.43 (s, 2H), 3.04-2.91 (m, 4H), 2.02 (s, 3H). |
| 3 | 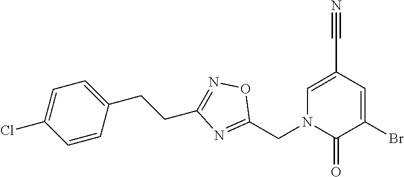 [SMILES: N#Cc1cc(Br)c(=O)n(Cc2nc(CCc3ccc(Cl)cc3)no2)c1] | 5-bromo-1-((3-(4- chlorophenethyl)- 1,2,4-oxadiazol-5- yl)methyl)-6-oxo- 1,6- dihydropyridine-3- carbonitrile | [M + H]⁺: 419, 421, 423 (3:3:1); ¹H NMR (300 MHz, DMSO-d₆) δ 8.83 (d, J = 2.27 Hz, 1H), 8.43 (d, J = 2.24 Hz, 1H), 7.35-7.27 (m, 2H), 7.26-7.17 (m, 2H), 5.50 (s, 2H), 3.06- 2.89 (m, 4H). |
| 5 | 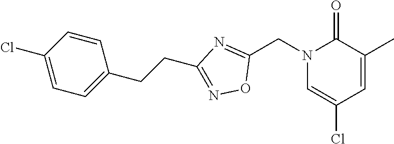 [SMILES: Cc1cc(Cl)cn(Cc2nc(CCc3ccc(Cl)cc3)no2)c1=O] | 5-chloro-1-((3-(4- chlorophenethyl)- 1,2,4-oxadiazol-5- yl)methyl)-3- methylpyridin- 2(1H)-one | [M + H]⁺: 364, 366 (3:2); ¹H NMR (300 MHz, CD₃OD) δ 7.79 (d, J = 2.78 Hz, 1H), 7.50-7.46 (m, 1H), 7.26-7.19 (m, 2H), 7.18-7.11 (m, 2H), 5.37 (s, 2H), 3.04-2.96 (m, 4H), 2.11 (s, 3H). |
| 6 | 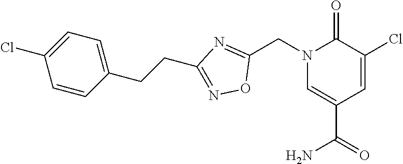 [SMILES: NC(=O)c1cc(Cl)c(=O)n(Cc2nc(CCc3ccc(Cl)cc3)no2)c1] | 5-chloro-1-((3-(4- chlorophenethyl)- 1,2,4-oxadiazol-5- yl)methyl)-6-oxo- 1,6- dihydropyridine-3- carboxamide | [M + H]⁺: 393, 395 (3:2); ¹H NMR (400 MHz, DMSO-d₆) δ 8.58 (d, J = 2.37 Hz, 1H), 8.34 (d, J = 2.37 Hz, 1H), 7.91 (s, 1H), 7.51 (s, 1H), 7.33-7.27 (m, 2H), 7.26-7.20 (m, 2H), 5.57 (s, 2H), 3.03-2.89 (m, 4H). |
| 7 | 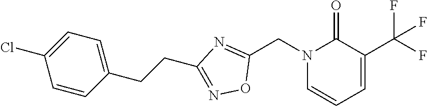 [SMILES: O=c1c(C(F)(F)F)cccn1Cc1nc(CCc2ccc(Cl)cc2)no1] | 1-((3-(4- chlorophenethyl)- 1,2,4-oxadiazol-5- yl)methyl)-3- (trifluoromethyl) pyridin-2(1H)-one | [M + H]⁺: 384, 386 (3:1); ¹H NMR (400 MHz, DMSO-d₆) δ 8.20 (dd, J = 6.88, 2.03 Hz, 1H), 8.09- 8.04 (m, 1H), 7.32-7.27 (m, 2H), 7.25-7.20 (m, 2H), 6.55-6.51 (m, 1H), |
| | | | 5.51 (s, 2H), 3.03-2.90 (m, |
| | | | 4H); ¹⁹F NMR (376 MHz, |
| | | | DMSO-d₆) δ −64.16 (s, 3F). |
| 8 | 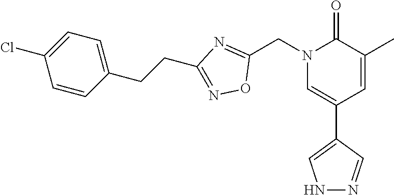 [SMILES: Cc1cc(-c2cn[nH]c2)cn(Cc2nc(CCc3ccc(Cl)cc3)no2)c1=O] | 1-((3-(4- chlorophenethyl)- 1,2,4-oxadiazol-5- yl)methyl)-3- methyl-5-(1H- pyrazol-4- yl)pyridin-2(1H)- one | [M + H]⁺: 396, 398 (3:1); ¹H NMR (300 MHz, DMSO-d₆) δ 8.02 (d, J = 2.50 Hz, 1H), 7.90 (s, 2H), 7.75 (d, J = 2.50 Hz, 1H), 7.34-7.19 (m, 4H), 5.39 (s, 2H), 3.04-2.91 (m, 4H), 2.06 (s, 3H). |
| 9 | 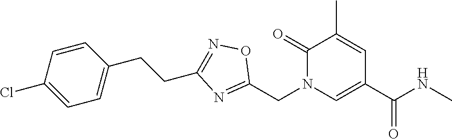 [SMILES: CNC(=O)c1cc(C)c(=O)n(Cc2nc(CCc3ccc(Cl)cc3)no2)c1] | 1-((3-(4- chlorophenethyl)- 1,2,4-oxadiazol-5- yl)methyl)-N,5- dimethyl-6-oxo- 1,6- dihydropyridine-3- carboxamide | [M + H]⁺: 387, 389 (3:1); ¹H NMR (300 MHz, DMSO-d₆) δ 8.36 (d, J = 2.47 Hz, 1H), 8.30-8.22 (m, 1H), 7.85 (s, 1H), 7.34- 7.26 (m, 2H), 7.26-7.18 (m, 2H), 5.48 (s, 2H), 3.04- 2.87 (m, 4H), 2.75 (d, J = 4.49 Hz, 3H), 2.03 (s, 3H). |
| 10 | 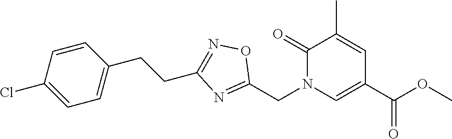 [SMILES: COC(=O)c1cc(C)c(=O)n(Cc2nc(CCc3ccc(Cl)cc3)no2)c1] | methyl 1-((3-(4- chlorophenethyl)- 1,2,4-oxadiazol-5- yl)methyl)-5- methyl-6-oxo-1,6- dihydropyridine-3- carboxylate | [M + H]⁺: 388, 390 (3:1); ¹H NMR (300 MHz, CD₃OD) δ 8.52-8.47 (m, 1H), 7.91-7.88 (m, 1H), 7.26-7.19 (m, 2H), 7.18- 7.10 (m, 2H), 5.46 (s, 2H), 3.87 (s, 3H), 3.00-2.98 (m, 4H), 2.13 (s, 3H). |
| 11 | 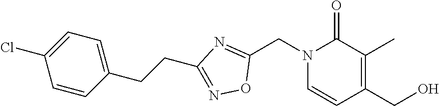 [SMILES: Cc1c(CO)ccn(Cc2nc(CCc3ccc(Cl)cc3)no2)c1=O] | 1-((3-(4- chlorophenethyl)- 1,2,4-oxadiazol-5- yl)methyl)-4- (hydroxymethyl)- 3-methylpyridin- 2(1H)-one | [M + H]⁺: 360, 362 (3:1); ¹H NMR (400 MHz, DMSO-d₆) δ 7.70-7.65 (m, 1H), 7.33-7.28 (m, 2H), 7.26-7.21 (m, 2H), 6.47 (d, J = 7.09 Hz, 1H), 5.37 (s, 2H), 5.34 (t, J = 5.58 Hz, |
| | | | 1H), 4.40 (d, J = 5.54 Hz, |
| | | | 2H), 3.01-2.90 (m, 4H), |
| | | | 1.91 (s, 3H). |
| 12 | 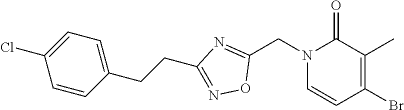 [SMILES: Cc1c(Br)ccn(Cc2nc(CCc3ccc(Cl)cc3)no2)c1=O] | 4-bromo-1-((3-(4- chlorophenethyl)- 1,2,4-oxadiazol-5- yl)methyl)-3- methylpyridin- 2(1H)-one | [M + H]⁺: 408, 410, 412 (2:3:1); ¹H NMR (400 MHz, DMSO-d₆) δ 7.68 (d, J = 7.34 Hz, 1H), 7.35-7.29 (m, 2H), 7.26-7.20 (m, 2H), 6.62 (d, J = 7.36 Hz, 1H), 5.39 (s, 2H), 3.02- |
| | | | 2.90 (m, 4H), 2.12 (s, 3H). |
| 13 | 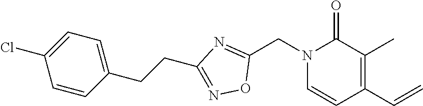 [SMILES: C=Cc1ccn(Cc2nc(CCc3ccc(Cl)cc3)no2)c(=O)c1C] | 1-((3-(4- chlorophenethyl)- 1,2,4-oxadiazol-5- yl)methyl)-3- methyl-4- vinylpyridin- 2(1H)-one | [M + H]⁺: 356, 358 (3:1); ¹H NMR (400 MHz, DMSO-d₆) δ 7.65 (d, J = 7.30 Hz, 1H), 7.33-7.26 (m, 2H), 7.26-7.22 (m, 2H), 6.93 (dd, J = 17.42, 11.04 Hz, 1H), 6.56 (d, J = 7.32 Hz, 1H), 5.96 (dd, J = |
| | | | 17.40, 1.07 Hz, 1H), 5.58 |
| | | | (dd, J = 11.10, 0.97 Hz, |
| | | | 1H), 5.37 (s, 2H), 3.03- |
| | | | 2.90 (m, 4H), 2.05 (s, 3H). |
| 14 | 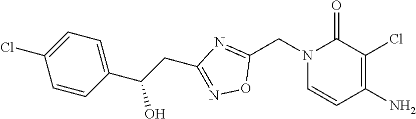 [SMILES: Nc1ccn(Cc2nc(C[C@H](O)c3ccc(Cl)cc3)no2)c(=O)c1Cl] | (S)-4-amino-3- chloro-1-((3-(2-(4- chlorophenyl)-2- hydroxyethyl)- 1,2,4-oxadiazol-5- yl)methyl)pyridin- 2(1H)-one | [M + H]+: 381, 383 (3:2); ¹H NMR (400 MHz, DMSO-d₆) δ 7.50 (d, J = 7.50 Hz, 1H), 7.42-7.32 (m, 4H), 6.57 (s, 2H), 5.98 (d, J = 7.49 Hz, 1H), 5.63 (d, J = 4.89 Hz, 1H), 5.29 (s, 2H), 5.00-4.90 (m, 1H), |
| | | | 3.04-2.89 (m, 2H). |
| 15 | 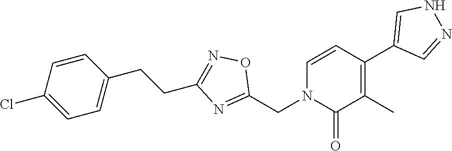 [SMILES: Cc1c(-c2cn[nH]c2)ccn(Cc2nc(CCc3ccc(Cl)cc3)no2)c1=O] | 1-((3-(4- chlorophenethyl)- 1,2,4-oxadiazol-5- yl)methyl)-3- methyl-4-(1H- pyrazol-4- yl)pyridin-2(1H)- one | [M + H]⁺: 396, 398 (3:1); ¹H NMR (400 MHz, DMSO-d₆) δ 8.04 (s, 2H), 7.67 (d, J = 7.22 Hz, 1H), 7.33-7.28 (m, 2H), 7.27- 7.22 (m, 2H), 6.51 (d, J = 7.18 Hz, 1H), 5.39 (s, 2H), 3.04-2.89 (m, 4H), 2.15 (s, 3H). |
| 16 | 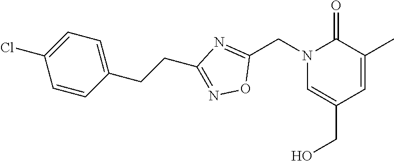 [SMILES: Cc1cc(CO)cn(Cc2nc(CCc3ccc(Cl)cc3)no2)c1=O] | 1-((3-(4- chlorophenethyl)- 1,2,4-oxadiazol-5- yl)methyl)-5- (hydroxymethyl)- 3-methylpyridin- 2(1H)-one | [M + H]⁺: 360, 362 (3:1); ¹H NMR (400 MHz, DMSO-d₆) δ 7.62-7.59 (m, 1H), 7.39-7.36 (m, 1H), 7.33-7.28 (m, 2H), 7.26- 7.21 (m, 2H), 5.38 (s, 2H), 5.17 (t, J = 5.47 Hz, 1H), 4.24 (d, J = 5.46 Hz, 2H), 3.03-2.89 (m, 4H), 2.00 (s, 3H). |
| 17 | 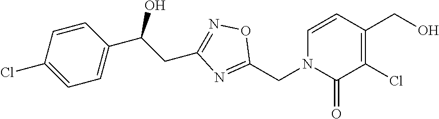 [SMILES: O=c1c(Cl)c(CO)ccn1Cc1nc(C[C@H](O)c2ccc(Cl)cc2)no1] | (S)-3-chloro-1-((3- (2-(4- chlorophenyl)-2- hydroxyethyl)- 1,2,4-oxadiazol-5- yl)methyl)-4- (hydroxymethyl) pyridin-2(1H)-one | [M + H]⁺: 396, 398 (3:2); ¹H NMR (400 MHz, DMSO-d₆) δ 7.88 (d, J = 7.12 Hz, 1H), 7.40-7.32 (m, 4H), 6.60 (d, J = 7.04 Hz, 1H), 5.68-5.60 (m, 2H), 5.49 (s, 2H), 4.99- 4.89 (m, 1H), 4.50 (d, J = |
| | | | 5.71 Hz, 2H), 3.04-2.90 |
| | | | (m, 2H). |
| 18 | 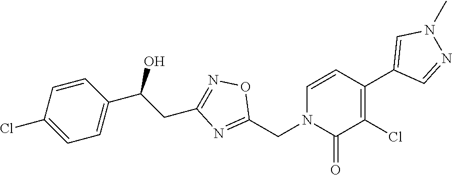 [SMILES: Cn1cc(-c2ccn(Cc3nc(C[C@H](O)c4ccc(Cl)cc4)no3)c(=O)c2Cl)cn1] | (S)-3-chloro-1-((3- (2-(4- chlorophenyl)-2- hydroxyethyl)- 1,2,4-oxadiazol-5- yl)methyl)-4-(1- methyl-1H- pyrazol-4- yl)pyridin-2(1H)- one | [M + H]⁺: 446, 448 (3:2); ¹H NMR (400 MHz, DMSO-d₆) δ 8.55 (s, 1H), 8.14 (s, 1H), 7.84 (d, J = 7.31 Hz, 1H), 7.39-7.30 (m, 4H), 6.75 (d, J = 7.32 Hz, 1H), 5.62 (d, J = 4.85 Hz, 1H), 5.47 (s, 2H), 4.99- 4.91 (m, 1H), 3.93 (s, 3H), 3.05-2.91 (m, 2H). |
| 19 | 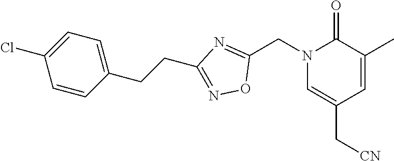 [SMILES: Cc1cc(CC#N)cn(Cc2nc(CCc3ccc(Cl)cc3)no2)c1=O] | 2-(1-((3-(4- chlorophenethyl)- 1,2,4-oxadiazol-5- yl)methyl)-5- methyl-6-oxo-1,6- dihydropyridin-3- yl)acetonitrile | [M + H]⁺: 369, 371 (3:1); ¹H NMR (300 MHz, CD₃OD) δ 7.66 (s, 1H), 7.48 (s, 1H), 7.27-7.19 (m, 2H), 7.19-7.11 (m, 2H), 5.40 (s, 2H), 3.72 (s, 2H), 3.04-2.94 (m, 4H), 2.13 (s, 3H). |
| 20 | 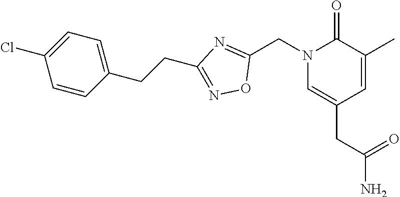 [SMILES: Cc1cc(CC(N)=O)cn(Cc2nc(CCc3ccc(Cl)cc3)no2)c1=O] | 2-(1-((3-(4- chlorophenethyl)- 1,2,4-oxadiazol-5- yl)methyl)-5- methyl-6-oxo-1,6- dihydropyridin-3- yl)acetamide | [M + H]⁺: 387, 389 (3:1); ¹H NMR (300 MHz, DMSO-d₆) δ 7.52 (s, 1H), 7.47 (s, 1H), 7.36-7.28 (m, 3H), 7.28-7.21 (m, 2H), 6.97 (s, 1H), 5.36 (s, 2H), 3.15 (s, 2H), 3.03-2.89 (m, 4H), 1.99 (s, 3H). |
| 21 | 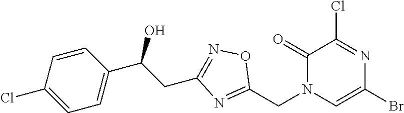 [SMILES: O=c1c(Cl)nc(Br)cn1Cc1nc(C[C@H](O)c2ccc(Cl)cc2)no1] | (S)-5-bromo-3- chloro-1-((3-(2-(4- chlorophenyl)-2- hydroxyethyl)- 1,2,4-oxadiazol-5- yl)methyl)pyrazin- 2(1H)-one | [M + H]⁺: 445, 447, 449 (3:5:2); ¹H NMR (300 MHz, DMSO-d₆) δ 8.32 (s, 1H), 7.43-7.30 (m, 4H), 5.64 (d, J = 4.79 Hz, 1H), 5.45 (s, 2H), 5.00-4.91 (m, 1H), 3.04-2.95 (m, 2H). |
| 22 | 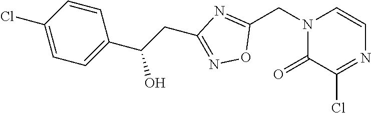 [SMILES: O=c1c(Cl)nccn1Cc1nc(C[C@H](O)c2ccc(Cl)cc2)no1] | (S)-3-chloro-1-((3- (2-(4- chlorophenyl)-2- hydroxyethyl)- 1,2,4-oxadiazol-5- yl)methyl)pyrazin- 2(1H)-one | [M + H]⁺: 367, 369 (3:2); ¹H NMR (400 MHz, DMSO-d₆) δ 7.91 (d, J = 4.31 Hz, 1H), 7.39-7.34 (m, 4H), 7.32 (d, J = 4.33 Hz, 1H), 5.63 (d, J = 4.84 Hz, 1H), 5.51 (s, 2H), 4.98- |
| | | | 4.91 (m, 1H), 3.06-2.93 |
| | | | (m, 2H). |
| 23 | 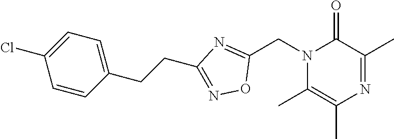 [SMILES: Cc1nc(C)c(=O)n(Cc2nc(CCc3ccc(Cl)cc3)no2)c1C] | 1-((3-(4- chlorophenethyl)- 1,2,4-oxadiazol-5- yl)methyl)-3,5,6- trimethylpyrazin- 2(1H)-one | [M + H]⁺: 359, 361 (3:1); ¹H NMR (400 MHz, CDCl₃) δ 7.25-7.21 (m, 2H), 7.13-7.08 (m, 2H), 5.44 (s, 2H), 3.03-2.99 (m, 4H), 2.56 (s, 3H), 2.43 (s, 3H), 2.30 (s, 3H). |
| 24 | 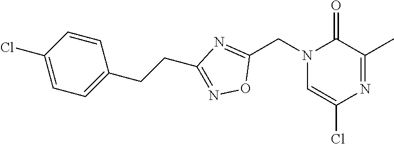 [SMILES: Cc1nc(Cl)cn(Cc2nc(CCc3ccc(Cl)cc3)no2)c1=O] | 5-chloro-1-((3-(4- chlorophenethyl)- 1,2,4-oxadiazol-5- yl)methyl)-3- methylpyrazin- 2(1H)-one | [M + H]⁺: 365, 367 (3:2); ¹H NMR (400 MHz, CD₃OD) δ 7.75 (s, 1H), 7.26-7.20 (m, 2H), 7.17- 7.13 (m, 2H), 5.35 (s, 2H), 3.03-2.98 (m, 4H), 2.39 (d, J = 0.86 Hz, 3H). |
| 25 | 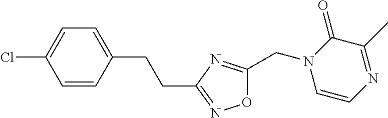 [SMILES: Cc1nccn(Cc2nc(CCc3ccc(Cl)cc3)no2)c1=O] | 1-((3-(4- chlorophenethyl)- 1,2,4-oxadiazol-5- yl)methyl)-3- methylpyrazin- 2(1H)-one | [M + H]⁺: 331, 333 (3:1); ¹H NMR (400 MHz, CD₃OD) δ 7.57-7.53 (m, 1H), 7.31 (d, J = 4.52 Hz, 1H), 7.27-7.22 (m, 2H), 7.20-7.14 (m, 2H), 5.40 (s, 2H), 3.05-3.00 (m, 4H), 2.42 (s, 3H). |
| 26 | 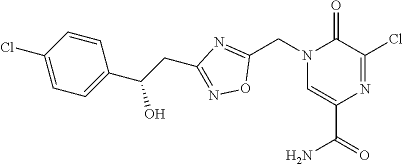 [SMILES: NC(=O)c1cn(Cc2nc(C[C@H](O)c3ccc(Cl)cc3)no2)c(=O)c(Cl)n1] | (S)-6-chloro-4-((3- (2-(4- chlorophenyl)-2- hydroxyethyl)- 1,2,4-oxadiazol-5- yl)methyl)-5-oxo- 4,5- dihydropyrazine-2- carboxamide | [M + H]⁺: 410, 412 (3:2); ¹H NMR (400 MHz, DMSO-d₆) δ 8.65 (s, 1H), 7.83 (s, 1H), 7.65 (s, 1H), 7.37-7.31 (m, 4H), 5.63 (d, J = 4.88 Hz, 1H), 5.60 (d, J = 2.20 Hz, 2H), 4.97-4.91 (m, 1H), 3.05-2.92 (m, 2H). |
| 27 | 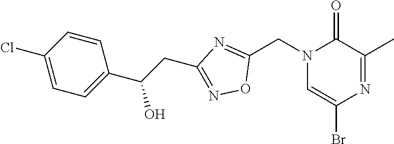 [SMILES: Cc1nc(Br)cn(Cc2nc(C[C@H](O)c3ccc(Cl)cc3)no2)c1=O] | (S)-5-bromo-1-((3- (2-(4- chlorophenyl)-2- hydroxyethyl)- 1,2,4-oxadiazol-5- yl)methyl)-3- methylpyrazin- 2(1H)-one | [M + H]⁺: 425, 427, 429 (3:3:1); ¹H NMR (400 MHz, DMSO-d₆) δ 8.09 (s, 1H), 7.39-7.33 (m, 4H), 5.64 (d, J = 4.83 Hz, 1H), 5.38 (s, 2H), 5.01-4.90 (m, 1H), 3.06-2.90 (m, 2H), 2.32 (s, 3H). |
| 28 | 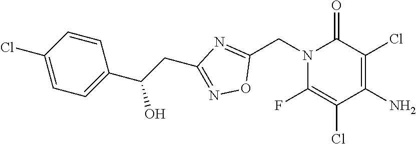 [SMILES: Nc1c(Cl)c(F)n(Cc2nc(C[C@H](O)c3ccc(Cl)cc3)no2)c(=O)c1Cl] | (S)-4-amino-3,5- dichloro-1-((3-(2- (4-chlorophenyl)- 2-hydroxyethyl)- 1,2,4-oxadiazol-5- yl)methyl)-6- fluoropyridin- 2(1H)-one | [M + H]⁺: 433, 435, 437 (3:3:1); ¹H NMR (400 MHz, DMSO-d₆) δ 7.35- 7,31 (m, 4H), 7.14 (s, 2H), 5.66 (d, J = 4.81 Hz, 1H), 5.62 (s, 2H), 5.03-4.93 (m, 1H), 3.10-2.92 (m, 2H); ¹⁹F NMR (282 MHz, DMSO- d₆) δ −77.60 (s, 1F). |
| 30 | 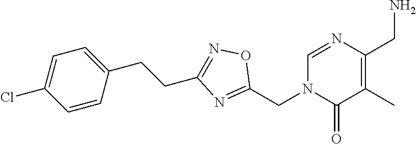 [SMILES: Cc1c(CN)ncn(Cc2nc(CCc3ccc(Cl)cc3)no2)c1=O] | 6-(aminomethyl)- 3-((3-(4- chlorophenethyl)- 1,2,4-oxadiazol-5- yl)methyl)-5- methylpyrimidin- 4(3H)-one | [M + H]⁺: 360, 362 (3:1); ¹H NMR (300 MHz, DMSO-d₆) δ 8.68 (s, 1H), 8.21 (s, 3H), 7.38-7.18 (m, 4H), 5.48 (s, 2H), 4.07 (s, 2H), 3.05-2.89 (m, 4H), 1.98 (s, 3H). |
| 31 | 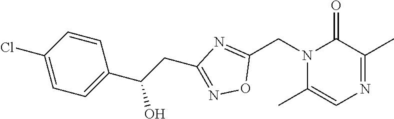 [SMILES: Cc1ncc(C)n(Cc2nc(C[C@H](O)c3ccc(Cl)cc3)no2)c1=O] | (S)-1-((3-(2-(4- chlorophenyl)-2- hydroxyethyl)- 1,2,4-oxadiazol-5- yl)methyl)-3,6- dimethylpyrazin- 2(1H)-one | [M + H]⁺: 361, 363 (3:1); ¹H NMR (300 MHz, CDCl₃) δ 7.35-7.31 (m, 4H), 7.15 (s, 1H), 5.39 (s, 2H), 5.15-5.12 (m, 1H), 3.22-3.06 (m, 2H), 2.46 (s, 3H), 2.30 (s, 3H). |
| 32 | 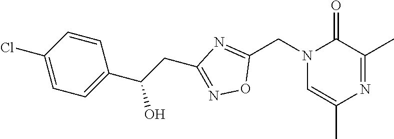 [SMILES: Cc1cn(Cc2nc(C[C@H](O)c3ccc(Cl)cc3)no2)c(=O)c(C)n1] | (S)-1-((3-(2-(4- chlorophenyl)-2- hydroxyethyl)- 1,2,4-oxadiazol-5- yl)methyl)-3,5- dimethylpyrazin- 2(1H)-one | [M + H]⁺: 361, 363 (3:1); ¹H NMR (400 MHz, DMSO-d₆) δ 7.36-7.32 (m, 4H), 7.19 (s, 1H), 5.63 (d, J = 4.75 Hz, 1H), 5.47 (s, 2H), 4.97-4.89 (m, 1H), 3.05-2.91 (m, 2H), 2.28 (d, J = 3.98 Hz, 6H). |
| 34 | 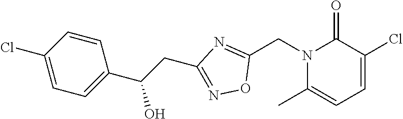 [SMILES: Cc1ccc(Cl)c(=O)n1Cc1nc(C[C@H](O)c2ccc(Cl)cc2)no1] | (S)-3-chloro-1-((3- (2-(4-chlorophenyl)- 2-hydroxyethyl)- 1,2,4-oxadiazol-5- yl)methyl)-6- methylpyridin- 2(1H)-one | [M + H]⁺: 380, 382 (3:2); ¹H NMR (400 MHz, DMSO-d₆) δ 7.74 (d, J = 7.56 Hz, 1H), 7.36-7.32 (m, 4H), 6.28 (d, J = 7.60 Hz, 1H), 5.63 (d, J = 4.73 Hz, 1H), 5.53 (s, 2H), 4.99- 4.89 (m, 1H), 3.06-2.92 |
| | | | (m, 2H), 2.37 (s, 3H). |
| 35 | 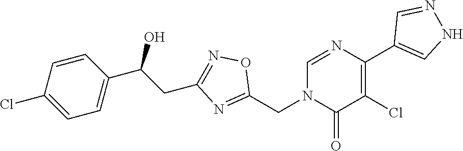 [SMILES: O=c1c(Cl)c(-c2cn[nH]c2)ncn1Cc1nc(C[C@H](O)c2ccc(Cl)cc2)no1] | (S)-5-chloro-3-((3- (2-(4- chlorophenyl)-2- hydroxyethyl)- 1,2,4-oxadiazol-5- yl)methyl)-6-(1H- pyrazol-4- yl)pyrimidin- 4(3H)-one | [M + H]⁺: 433, 435 (3:2); ¹H NMR (400 MHz, DMSO-d₆) δ 13.50 (s, 1H), 8.70-8.11 (m, 3H), 7.43- 7.28 (m, 4H), 5.62 (d, J = 4.81 Hz, 1H), 5.56-5.43 (m, 2H), 4.99-4.90 (m, 1H), 3.08-2.90 (m, 2H). |
| 36 | 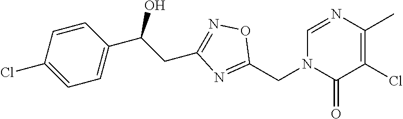 [SMILES: Cc1ncn(Cc2nc(C[C@H](O)c3ccc(Cl)cc3)no2)c(=O)c1Cl] | (S)-5-chloro-3-((3- (2-(4- chlorophenyl)-2- hydroxyethyl)- 1,2,4-oxadiazol-5- yl)methyl)-6- methylpyrimidin- 4(3H)-one | [M + H]⁺: 381, 383 (3:2); ¹H NMR (300 MHz, DMSO-d₆) δ 8.58 (s, 1H), 7.37-7.33 (m, 4H), 5.63 (d, J = 4.80 Hz, 1H), 5.49 (s, 2H), 4.98-4.89 (m, 1H), 3.03-2.95 (m, 2H), 2.41 (s, 3H). |

The compounds in Table 2 below can be prepared by using the procedures described in Schemes 1-8 or in an analogous fashion to that described for Compound 4, 29, 33, or 63 in Examples 2-5.

TABLE 2

| Compound | |
|---|---|
| No. | Chemical structure |
| 38 | 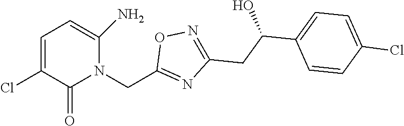 [SMILES: Nc1ccc(Cl)c(=O)n1Cc1nc(C[C@H](O)c2ccc(Cl)cc2)no1] |
| 40 | 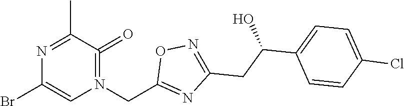 |
| 41 | 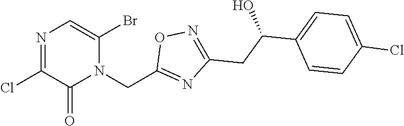 [SMILES: O=c1c(Cl)ncc(Br)n1Cc1nc(C[C@H](O)c2ccc(Cl)cc2)no1] |
| 42 | 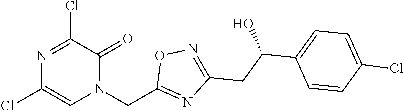 [SMILES: O=c1c(Cl)nc(Cl)cn1Cc1nc(C[C@H](O)c2ccc(Cl)cc2)no1] |
| 43 | 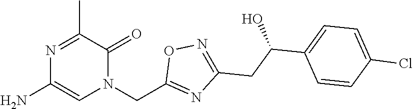 [SMILES: Cc1nc(N)cn(Cc2nc(C[C@H](O)c3ccc(Cl)cc3)no2)c1=O] |
| 44 | 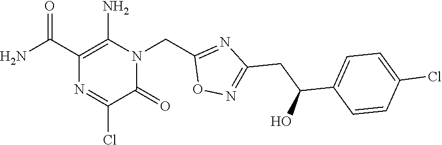 [SMILES: NC(=O)c1nc(Cl)c(=O)n(Cc2nc(C[C@H](O)c3ccc(Cl)cc3)no2)c1N] |
| 47 | 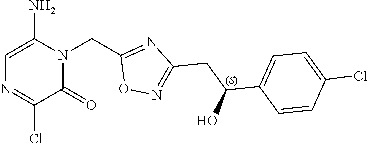 |
| 48 | 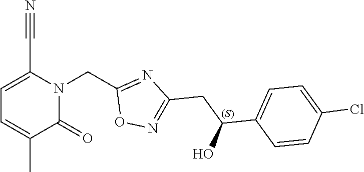 |
| 49 | 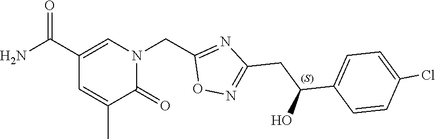 |
| 51 | 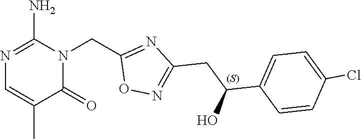 |
| 52 | 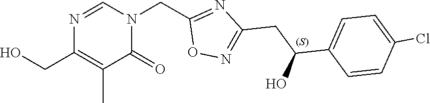 |
| 53 | 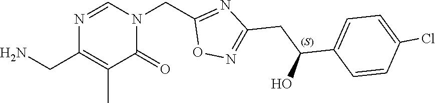 |
| 55 | 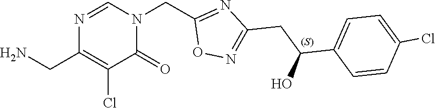 |
| 56 | 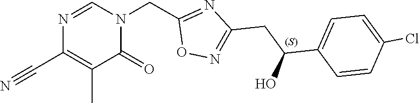 |
| 57 | 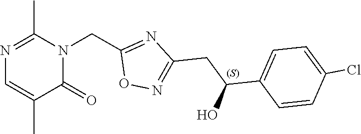 |
| 59 | 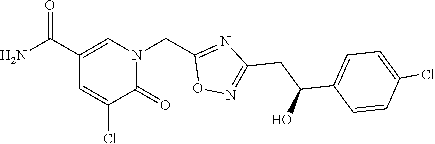 [SMILES: NC(=O)c1cc(Cl)c(=O)n(Cc2nc(C[C@H](O)c3ccc(Cl)cc3)no2)c1] |
| 60 | 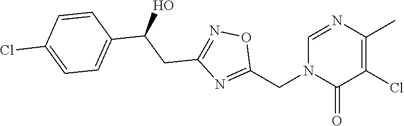 [SMILES: Cc1ncn(Cc2nc(C[C@H](O)c3ccc(Cl)cc3)no2)c(=O)c1Cl] |
| 61 | 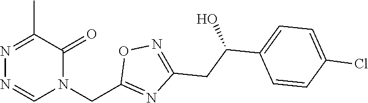 [SMILES: Cc1nncn(Cc2nc(C[C@H](O)c3ccc(Cl)cc3)no2)c1=O] |

The compounds in Table 3 below can be prepared by using the procedure described in Schemes 1-8 or in an analogous fashion to that described for Compounds 4, 29, 33, or 63 in Examples 2-5.

TABLE 3

| Compound | | | |
|---|---|---|---|
| No | Structure | Chemical Name | MS: (M + H)⁺ & ¹H MNR |
| 54 | 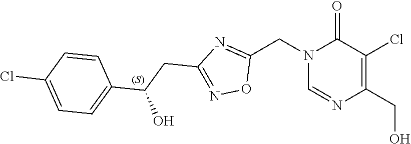 | (S)-5-chloro-3-((3- (2-(4- chlorophenyl)-2- hydroxyethyl)- 1,2,4-oxadiazol-5- yl)methyl)-6- (hydroxymethyl) pyrimidin-4(3H)- one | [M + H]⁺: 397, 399 (3:2); ¹H NMR (400 MHz, DMSO- d₆) δ 8.69 (s, 1H), 7.41-7.31 (m, 4H), 5.63 (t, J = 4.66 Hz, 1H), 5.53 (s, 2H), 5.50-5.43 (m, 1H), 4.98-4.91 (m, 1H), 4.49 (d, J = 4.55 Hz, 2H), 3.06-2.92 (m, 2H). |
| 58 | 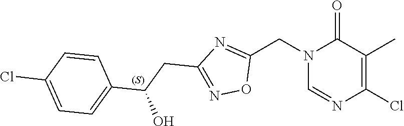 | (S)-6-chloro-3-((3- (2-(4- chlorophenyl)-2- hydroxyethyl)- 1,2,4-oxadiazol-5- yl)methyl)-5- methylpyrimidin- 4(3H)-one | [M + H]⁺: 381, 383 (3:2); ¹H NMR (300 MHz, DMSO- d₆) δ 8.56 (s, 1H), 7.41-7.27 (m, 4H), 5.62 (d, J = 4.80 Hz, 1H), 5.45 (s, 2H), 4.99- 4.90 (m, 1H), 3.06-2.91 (m, 2H), 2.07 (s, 3H). |
| 62 | 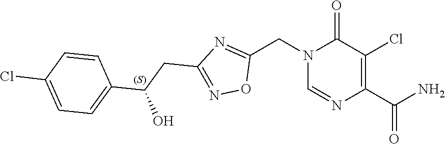 | (S)-5-chloro-1-((3- (2-(4- chlorophenyl)-2- hydroxyethyl)- 1,2,4-oxadiazol-5- yl)methyl)-6-oxo- 1,6- dihydropyrimidine- 4-carboxamide | [M + H]⁺: 410, 412 (3:2); ¹H NMR (300 MHz, DMSO- d₆) δ 8.72 (s, 1H), 8.17 (s, 1H), 7.92 (s, 1H), 7.44-7.30 (m, 4H), 5.64 (d, J = 4.78 Hz, 1H), 5.55 (s, 2H), 5.01- 4.90 (m, 1H), 3.09-2.91 (m, 2H). |
| 64 | 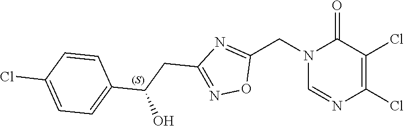 | (S)-5,6-dichloro-3- ((3-(2-(4- chlorophenyl)-2- hydroxyethyl)- 1,2,4-oxadiazol-5- yl)methyl) pyrimidin- 4(3H)-one | [M − H]⁻: 399, 401, 403 (3:3:1); ¹H NMR (300 MHz, DMSO-d₆) δ 8.72 (s, 1H), 7.42-7.27 (m, 4H), 5.64 (d, J = 4.78 Hz, 1H), 5.53 (s, 2H), 5.00-4.91 (m, 1H), 3.07-2.92 (m, 2H). |
| 65 | 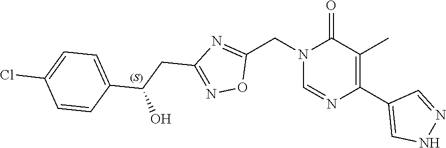 | (S)-3-((3-(2-(4- chlorophenyl)-2- hydroxyethyl)- 1,2,4-oxadiazol-5- yl)methyl)-5- methyl-6-(1H- pyrazol-4- yl)pyrimidin- 4(3H)-one | [M + H]⁺: 413, 415 (3:1); ¹H NMR (400 MHz, DMSO- d₆) δ 13.32 (s, 1H), 8.50 (s, 1H), 8.17-8.15 (m, 2H), 7.42-7.25 (m, 4H), 5.63 (s, 1H), 5.43 (s, 2H), 5.06-4.83 (m, 1H), 3.10-2.85 (m, 2H), 2.18 (s, 3H). |
| 66 | 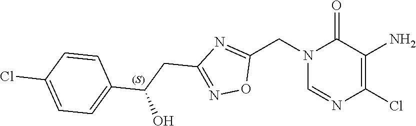 | (S)-5-amino-6- chloro-3-((3-(2-(4- chlorophenyl)-2- hydroxyethyl)- 1,2,4-oxadiazol-5- yl)methyl) pyrimidin- 4(3H)-one | [M + H]⁺: 382, 384 (3:2); ¹H NMR (300 MHz, DMSO- d₆) δ 7.98 (s, 1H), 7.37-7.33 (m, 4H), 5.62 (d, J = 4.82 Hz, 1H), 5.50 (s, 2H), 5.47 (s, 2H), 5.01-4.88 (m, 1H), 3.05-2.87 (m, 2H). |
| 67 | 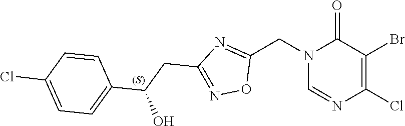 | (S)-5-bromo-6- chloro-3-((3-(2-(4- chlorophenyl)-2- hydroxyethyl)- 1,2,4-oxadiazol-5- yl)methyl) pyrimidin- 4(3H)-one | [M + H]⁺: 445, 447, 449 (3:3:2); ¹H NMR (400 MHz, DMSO-d₆) δ 8.73 (s, 1H), 7.36-7.33 (m, 4H), 5.63 (s, 1H), 5.58-5.46 (m, 2H), 4.97-4.95 (m, 1H), 3.08-2.90 (m, 2H). |
| 68 | 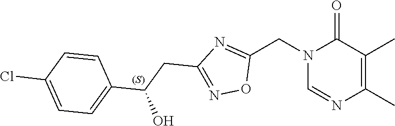 | (S)-3-((3-(2-(4- chlorophenyl)-2- hydroxyethyl)- 1,2,4-oxadiazol-5- yl)methyl)-5,6- dimethylpyrimidin- 4(3H)-one | [M + H]⁺: 361, 363 (3:1); ¹H NMR (300 MHz, DMSO- d₆) δ 8.41 (s, 1H), 7.39-7.30 (m, 4H), 5.62 (d, J = 4.82 Hz, 1H), 5.40 (s, 2H), 5.00- 4.88 (m, 1H), 3.06-2.90 (m, 2H), 2.26 (s, 3H), 1.95 (s, 3H). |
| 69 | 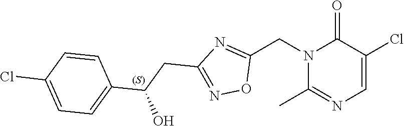 | (S)-5-chloro-3-((3- (2-(4- chlorophenyl)-2- hydroxyethyl)- 1,2,4-oxadiazol-5- yl)methyl)-2- methylpyrimidin- 4(3H)-one | [M + H]⁺: 381, 383 (3:2); ¹H NMR (300 MHz, DMSO- d₆) δ 8.23 (s, 1H), 7.35-7.32 (m, 4H), 5.62 (d, J = 4.74 Hz, 1H), 5.57 (s, 2H), 4.99- 4.90 (m, 1H), 3.08-2.92 (m, 2H), 2.53 (s, 3H). |
| 70 | 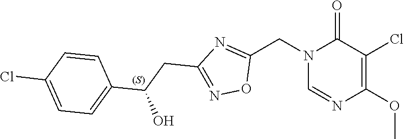 | (S)-5-chloro-3-((3- (2-(4- chlorophenyl)-2- hydroxyethyl)- 1,2,4-oxadiazol-5- yl)methyl)-6- methoxypyrimidin- 4(3H)-one | [M + H]⁺: 397, 399 (3:2); ¹H NMR (300 MHz, DMSO- d₆) δ 8.68 (s, 1H), 7.37-7.33 (m, 4H), 5.63 (d, J = 4.80 Hz, 1H), 5.49 (s, 2H), 4.99- 4.89 (m, 1H), 4.01 (s, 3H), 3.07-2.89 (m, 2H). |
| 71 | 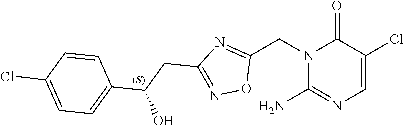 | (S)-2-amino-5- chloro-3-((3-(2-(4- chlorophenyl)-2- hydroxyethyl)- 1,2,4-oxadiazol-5- yl)methyl) pyrimidin- 4(3H)-one | [M + H]⁺: 382, 384 (3 : 2); ¹H NMR (400 MHz, DMSO- d₆) δ 7.90 (s, 1H), 7.64 (s, 2H), 7.40-7.33 (m, 4H), 5.64 (d, J = 4.82 Hz, 1H), 5.48- 5.35 (m, 2H), 5.00-4.91 (m, 1H), 3.07-2.92 (m, 2H). |
| 72 | 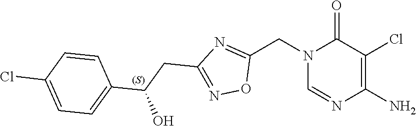 | (S)-6-amino-5- chloro-3-((3-(2-(4- chlorophenyl)-2- hydroxyethyl)- 1,2,4-oxadiazol-5- yl)methyl) pyrimidin- 4(3H)-one | [M + H]⁺: 382, 384 (3:2); ¹H NMR (300 MHz, DMSO- d₆) δ 8.31 (s, 1H), 7.41-7.30 (m, 4H), 7.15 (s, 2H), 5.63 (d, J = 4.86 Hz, 1H), 5.34 (s, 2H), 5.02-4.88 (m, 1H), 3.05-2.87 (m, 2H). |
| 73 | 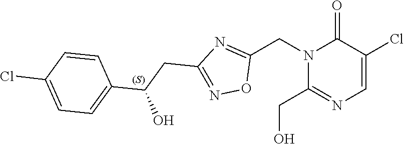 | (S)-5-chloro-3-((3- (2-(4- chlorophenyl)-2- hydroxyethyl)- 1,2,4-oxadiazol-5- yl)methyl)-2- (hydroxymethyl) pyrimidin-4(3H)- one | [M − H]⁻: 395, 397 (3:2); ¹H NMR (300 MHz, DMSO-d₆) δ 8.33 (s, 1H), 7.35-7.32 (m, 4H), 5.93 (t, J = 6.01 Hz, 1H), 5.63 (d, J = 4.78 Hz, 1H), 5.58 (s, 2H), 4.98-4.89 (m, 1H), 4.55 (d, J = 5.98 Hz, 2H), 3.06-2.88 (m, 2H). |
| 74 | 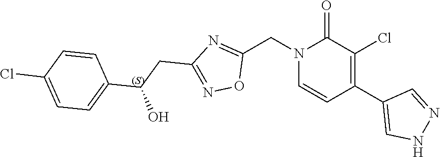 | (S)-3-chloro-1-((3- (2-(4- chlorophenyl)-2- hydroxyethyl)- 1,2,4-oxadiazol-5- yl)methyl)-4-(1H- pyrazol-4- yl)pyridin-2(1H)- one | [M + H]⁺: 432, 434 (3:2); ¹H NMR (300 MHz, DMSO- d₆) δ 13.43 (s, 1H), 8.64-8.03 (m, 2H), 7.84 (d, J = 7.30 Hz, 1H), 7.40-7.31 (m, 4H), 6.79 (d, J = 7.29 Hz, 1H), 5.63 (d, J = 4.85 Hz, 1H), 5.47 (s, 2H), 5.00-4.90 (m, 1H), 3.07-2.88 (m, 2H) |

### Example 6. Evaluation of TRPA1 Inhibitor Activities

This assay was used to evaluate the disclosed compounds' inhibition activities against the human TRPA1 channel.

### Cell Culture

CHO cells inducibly expressing human TRPA1 were grown in DMEM containing 10% heat-inactivated FBS, 1 mM Sodium Pyruvate, 2 mM L-Glutamine, Zeocin (100 μg/ml) and Blasticidin (10 μg/ml). Expression was induced by addition of Doxycycline (1 μg/ml) 24 hours before experiments. Cells used for electrophysiology were plated in plastic culture flasks and grown at 37° C. in a 5% CO₂-humidified tissue culture incubator per ChanPharm SOP. Stocks were maintained in cryogenic storage.

### Solutions

The cells were bathed in an extracellular solution containing 80 mM NaCl, 60 mM NMDG, 4 mM KCl, 2 mM CaCl₂), 6 mM MgCl₂, 5 mM Glucose. 10 mM HEPES, 3 mM HEDTA; pH adjusted to 7.4 with NaOH; 305-310 mOsm. All compounds were dissolved in DMSO at 30 mM. The internal solution contained 10 mM CsCl, 110 mM CsF, 10 mM NaCl, 10 mM EGTA, 10 mM HEPES, 4 mM MgATP, 0.25 mM NaGTP, 4 mM BAPTA; pH adjusted to 7.2 with CsOH; 285-290 mOsm. Compound stock solutions were freshly diluted with external solution to concentrations of 3 nM, 10 nM 30 nM, 100 nM, 300 nM, 1 μM, 3 μM, 10 μM, and 30 μM. The highest content of DMSO (0.1%) was present at 30 μM.

### Patch Clamp Recordings and Compound Application

All experiments were performed at room temperature. Each cell acted as its own control. In preparation for a current recording session, intracellular solution (see above) was loaded into the intracellular compartments of the automated patch clamp platform SyncroPatch (Nanion) chip and the cell suspension was pipetted into the extracellular compartments. After establishment of a whole-cell configuration, membrane current recordings and compound application were enabled by means of the SyncroPatch. TRPA1 currents were elicited by application of carvacrol (300 μM) at a constant holding potential of −60 mV (see Table A below).

### Data Analysis

To determine IC₅₀ values, AUC and peak values, obtained in the presence of a given compound concentration, were normalized to control values in absence of compound. Using DataControl384 (Nanion's proprietary software), IC₅₀ values were derived by fitting the normalized data to the Hill equation.

### Example 7. Evaluation of hERG Activities

This assay was used to evaluate the disclosed compounds' inhibition activities against the hERG channel.

### Cell Culture

CHO-K1 cells stably expressing hERG were grown in Ham's F-12 Medium with Glutamine containing 10% heat-inactivated FBS, 1% Penicillin/Streptomycin, Hygromycin (100 μg/ml), and G418 (100 μg/ml). Cells used for electrophysiology were plated in plastic culture flasks and grown at 37° C. in a 5% CO₂-humidified incubator per ChanPharm SOP. Stocks were maintained in cryogenic storage.

### Solutions

The cells were bathed in an extracellular solution containing 140 mM NaCl, 4 mM KCl, 2 mM CaCl₂), 1 mM MgCl₂, 5 mM Glucose, and 10 mM HEPES; pH adjusted to 7.4 with NaOH; 295-305 mOsm. The internal solution contained 10 mM KCl, 110 mM KF, 10 mM NaCl, 10 mM EGTA, 10 mM HEPES; pH adjusted to 7.2 with KOH; 280-285 mOsm. All compounds were dissolved in DMSO at 30 mM. Compound stock solutions were freshly diluted with external solution to concentrations of 50 μM and 100 μM. The highest content of DMSO (0.15%) was present at 50 μM.

### Voltage Protocol

All experiments were performed at room temperature. Each cell acted as its own control. In preparation for a recording session, intracellular solution (see above) was loaded into the intracellular compartments of the automated patch clamp platform SyncroPatch (Nanion) chip and the cell suspension was pipetted into the extracellular compartments. After establishment of a whole-cell configuration, membrane current recordings, and compound application were enabled by means of the SyncroPatch. hERG currents were elicited by a voltage pulse pattern with fixed amplitudes (depolarization: +20 mV amplitude, 300 ms duration; repolarization: −50 mV, 300 ms duration) repeated at 3 s intervals from a holding potential of −80 mV.

### Data Analysis

Data acquisition and analysis were performed using DataControl384 (Nanion's proprietary software). To determine the (percentage) inhibition, the last single pulse in the pulse train (i.e., the repolarization step to −50 mV; tail current) at a given compound concentration was used. AUC and peak values, obtained in the presence of compound, were normalized to control values in the absence of compound.

Table 4 provides a summary of the inhibition activities of certain selected compounds of the instant invention against TRPA1 channel and hERG channel.

TABLE 4
IC₅₀ (μM) values of certain exemplified compounds against
TRPA1 channel and hERG channel

| Compound | | TrpA1 IC₅₀ | hERG |
|---|---|---|---|
| No. | Chemical structure | (μM) | IC₅₀ (μM) |
| 1 | 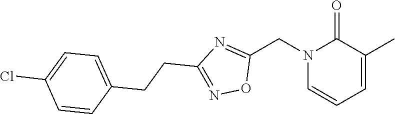 [SMILES: Cc1cccn(Cc2nc(CCc3ccc(Cl)cc3)no2)c1=O] | <10 | * |
| 2 | 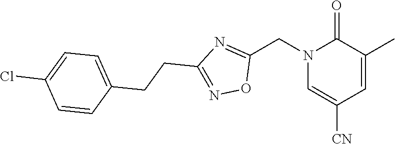 [SMILES: Cc1cc(C#N)cn(Cc2nc(CCc3ccc(Cl)cc3)no2)c1=O] | <1 | * |
| 3 | 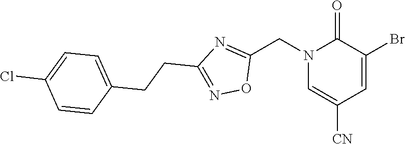 [SMILES: N#Cc1cc(Br)c(=O)n(Cc2nc(CCc3ccc(Cl)cc3)no2)c1] | <1 | * |
| 4 | 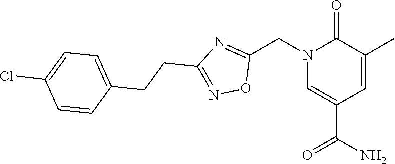 [SMILES: Cc1cc(C(N)=O)cn(Cc2nc(CCc3ccc(Cl)cc3)no2)c1=O] | <1 | >50 |
| 5 | 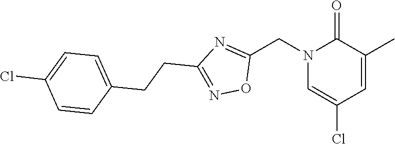 [SMILES: Cc1cc(Cl)cn(Cc2nc(CCc3ccc(Cl)cc3)no2)c1=O] | <3 | * |
| 6 | 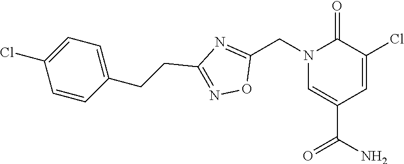 [SMILES: NC(=O)c1cc(Cl)c(=O)n(Cc2nc(CCc3ccc(Cl)cc3)no2)c1] | <0.3 | <50 |
| 7 | 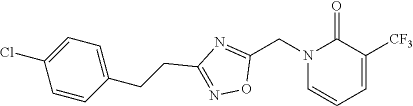 [SMILES: O=c1c(C(F)(F)F)cccn1Cc1nc(CCc2ccc(Cl)cc2)no1] | <10 | * |
| 8 | 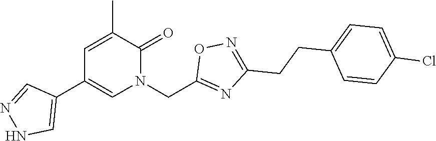 [SMILES: Cc1cc(-c2cn[nH]c2)cn(Cc2nc(CCc3ccc(Cl)cc3)no2)c1=O] | <10 | <50 |
| 9 | 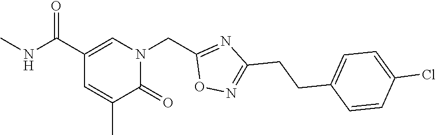 [SMILES: CNC(=O)c1cc(C)c(=O)n(Cc2nc(CCc3ccc(Cl)cc3)no2)c1] | <1 | * |
| 10 | 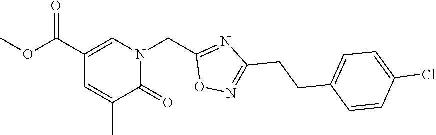 [SMILES: COC(=O)c1cc(C)c(=O)n(Cc2nc(CCc3ccc(Cl)cc3)no2)c1] | <3 | * |
| 11 | 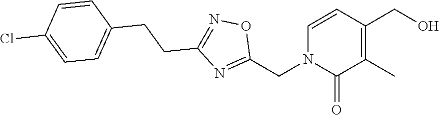 [SMILES: Cc1c(CO)ccn(Cc2nc(CCc3ccc(Cl)cc3)no2)c1=O] | <3 | * |
| 12 | 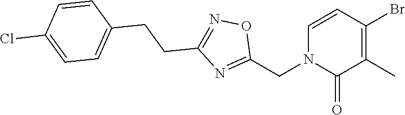 [SMILES: Cc1c(Br)ccn(Cc2nc(CCc3ccc(Cl)cc3)no2)c1=O] | <3 | * |
| 13 | 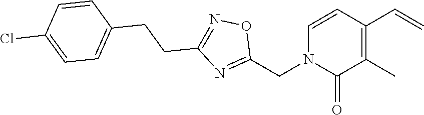 [SMILES: C=Cc1ccn(Cc2nc(CCc3ccc(Cl)cc3)no2)c(=O)c1C] | <3 | * |
| 14 | 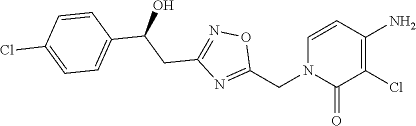 [SMILES: Nc1ccn(Cc2nc(C[C@H](O)c3ccc(Cl)cc3)no2)c(=O)c1Cl] | <3 | >50 |
| 15 | 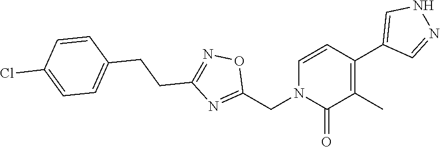 [SMILES: Cc1c(-c2cn[nH]c2)ccn(Cc2nc(CCc3ccc(Cl)cc3)no2)c1=O] | <3 | * |
| 16 | 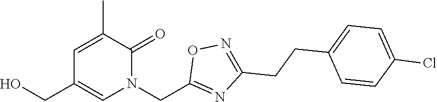 [SMILES: Cc1cc(CO)cn(Cc2nc(CCc3ccc(Cl)cc3)no2)c1=O] | <3 | * |
| 17 | 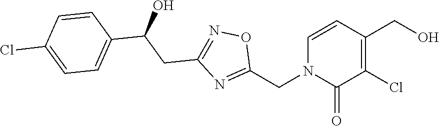 [SMILES: O=c1c(Cl)c(CO)ccn1Cc1nc(C[C@H](O)c2ccc(Cl)cc2)no1] | <1 | >50 |
| 18 | 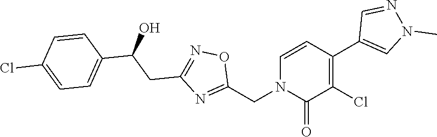 [SMILES: Cn1cc(-c2ccn(Cc3nc(C[C@H](O)c4ccc(Cl)cc4)no3)c(=O)c2Cl)cn1] | <1 | <50 |
| 19 | 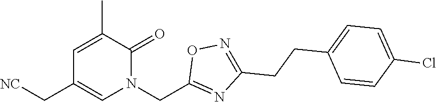 [SMILES: Cc1cc(CC#N)cn(Cc2nc(CCc3ccc(Cl)cc3)no2)c1=O] | <1 | * |
| 20 | 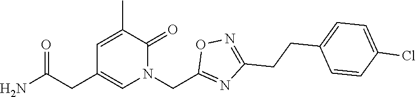 [SMILES: Cc1cc(CC(N)=O)cn(Cc2nc(CCc3ccc(Cl)cc3)no2)c1=O] | <10 | * |
| 21 | 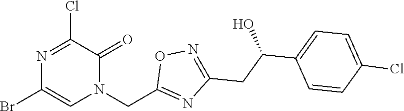 [SMILES: O=c1c(Cl)nc(Br)cn1Cc1nc(C[C@H](O)c2ccc(Cl)cc2)no1] | <0.3 | * |
| 22 | 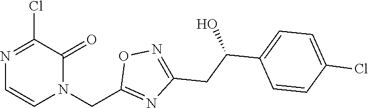 [SMILES: O=c1c(Cl)nccn1Cc1nc(C[C@H](O)c2ccc(Cl)cc2)no1] | <0.3 | <50 |
| 23 | 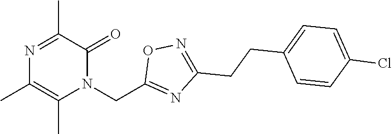 [SMILES: Cc1nc(C)c(=O)n(Cc2nc(CCc3ccc(Cl)cc3)no2)c1C] | <1 | * |
| 24 | 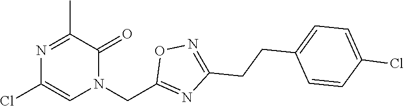 [SMILES: Cc1nc(Cl)cn(Cc2nc(CCc3ccc(Cl)cc3)no2)c1=O] | <1 | * |
| 25 | 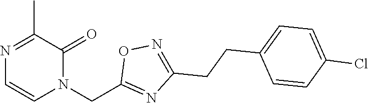 [SMILES: Cc1nccn(Cc2nc(CCc3ccc(Cl)cc3)no2)c1=O] | <3 | * |
| 26 | 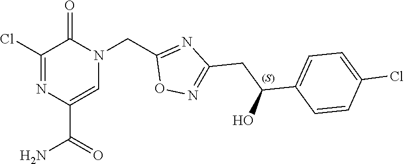 | <1 | >50 |
| 27 | 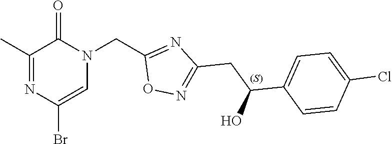 | <0.3 | <50 |
| 28 | 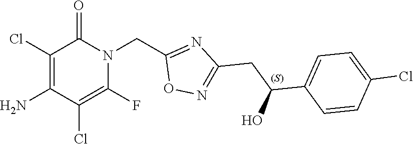 | <1 | * |
| 29 | 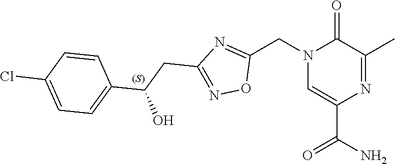 | <0.3 | >50 |
| 30 | 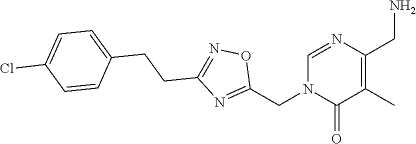 [SMILES: Cc1c(CN)ncn(Cc2nc(CCc3ccc(Cl)cc3)no2)c1=O] | <0.3 | <50 |
| 31 | 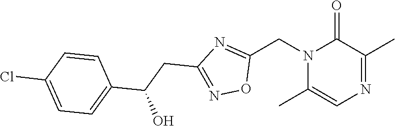 [SMILES: Cc1ncc(C)n(Cc2nc(C[C@H](O)c3ccc(Cl)cc3)no2)c1=O] | <1 | * |
| 32 | 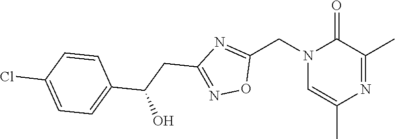 [SMILES: Cc1cn(Cc2nc(C[C@H](O)c3ccc(Cl)cc3)no2)c(=O)c(C)n1] | <1 | * |
| 33 | 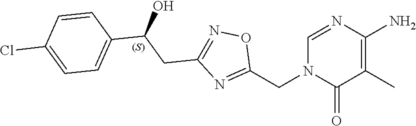 | <1 | * |
| 34 | 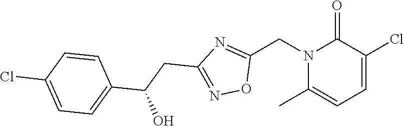 [SMILES: Cc1ccc(Cl)c(=O)n1Cc1nc(C[C@H](O)c2ccc(Cl)cc2)no1] | <1 | * |
| 35 | 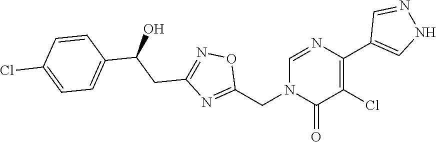 [SMILES: O=c1c(Cl)c(-c2cn[nH]c2)ncn1Cc1nc(C[C@H](O)c2ccc(Cl)cc2)no1] | <0.3 | <50 |
| 36 | 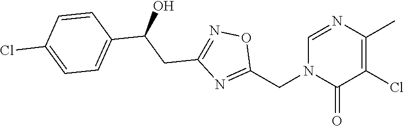 [SMILES: Cc1ncn(Cc2nc(C[C@H](O)c3ccc(Cl)cc3)no2)c(=O)c1Cl] | <0.1 | >50 |

*not determined (ND)

Table 5 provides a summary of the inhibition activities of certain selected compounds of the instant invention against TRPA1 channel and hERG channel.

TABLE 5
IC₅₀ (UM) values of certain exemplified compounds against
TRPA1 channel and hERG channel

| Compound | | TrpA1 | hERG |
|---|---|---|---|
| No. | Chemical structure | IC₅₀ (μM) | IC₅₀ (μM) |
| 54 | 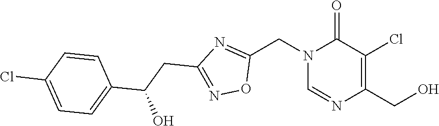 [SMILES: O=c1c(Cl)c(CO)ncn1Cc1nc(C[C@H](O)c2ccc(Cl)cc2)no1] | <0.3 | >50 |
| | 5-chloro-3-({3-[(2S)-2-(4-chlorophenyl)- | | |
| | 2-hydroxyethyl]-1,2,4-oxadiazol-5- | | |
| | yl}methyl)-6-(hydroxymethyl)-3,4- | | |
| | dihydropyrimidin-4-one | | |
| 58 | 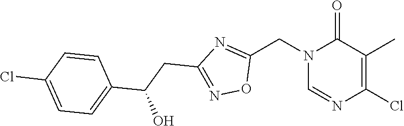 [SMILES: Cc1c(Cl)ncn(Cc2nc(C[C@H](O)c3ccc(Cl)cc3)no2)c1=O] | <0.1 | >50 |
| | 6-chloro-3-({3-[(2S)-2-(4-chlorophenyl)- | | |
| | 2-hydroxyethyl]-1,2,4-oxadiazol-5- | | |
| | yl}methyl)-5-methyl-3,4- | | |
| | dihydropyrimidin-4-one | | |
| 62 | 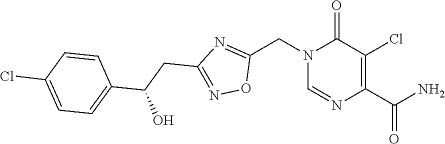 [SMILES: NC(=O)c1ncn(Cc2nc(C[C@H](O)c3ccc(Cl)cc3)no2)c(=O)c1Cl] | <1 | * |
| | 5-chloro-1-({3-[(2S)-2-(4-chlorophenyl)- | | |
| | 2-hydroxyethyl]-1,2,4-oxadiazol-5- | | |
| | yl}methyl)-6-oxo-1,6-dihydropyrimidine- | | |
| | 4-carboxamide | | |
| 63 | 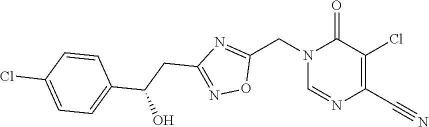 [SMILES: N#Cc1ncn(Cc2nc(C[C@H](O)c3ccc(Cl)cc3)no2)c(=O)c1Cl] | <0.1 | <50 |
| | 5-chloro-1-({3-[(2S)-2-(4-chlorophenyl)- | | |
| | 2-hydroxyethyl]-1,2,4-oxadiazol-5- | | |
| | yl}methyl)-6-oxo-1,6-dihydropyrimidine- | | |
| | 4-carbonitrile | | |
| 64 | 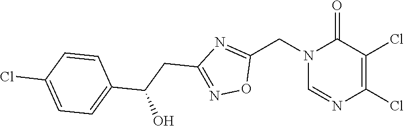 [SMILES: O=c1c(Cl)c(Cl)ncn1Cc1nc(C[C@H](O)c2ccc(Cl)cc2)no1] | <0.1 | * |
| | 5,6-dichloro-3-({3-[(2S)-2-(4- | | |
| | chlorophenyl)-2-hydroxyethyl]-1,2,4- | | |
| | oxadiazol-5-yl}methyl)-3,4- | | |
| | dihydropyrimidin-4-one | | |
| 65 | 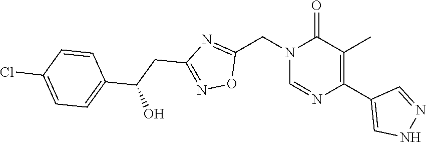 [SMILES: Cc1c(-c2cn[nH]c2)ncn(Cc2nc(C[C@H](O)c3ccc(Cl)cc3)no2)c1=O] | <0.3 | <50 |
| | 3-({3-[(2S)-2-(4-chlorophenyl)-2- | | |
| | hydroxyethyl]-1,2,4-oxadiazol-5- | | |
| | yl}methyl)-5-methyl-6-(1H-pyrazol-4-yl)- | | |
| | 3,4-dihydropyrimidin-4-one | | |
| 66 | 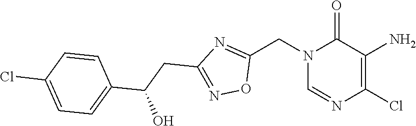 [SMILES: Nc1c(Cl)ncn(Cc2nc(C[C@H](O)c3ccc(Cl)cc3)no2)c1=O] | <0.3 | >50 |
| | 5-amino-6-chloro-3-({3-[(2S)-2-(4- | | |
| | chlorophenyl)-2-hydroxyethyl]-1,2,4- | | |
| | oxadiazol-5-yl}methyl)-3,4- | | |
| | dihydropyrimidin-4-one | | |
| 67 | 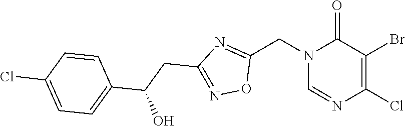 [SMILES: O=c1c(Br)c(Cl)ncn1Cc1nc(C[C@H](O)c2ccc(Cl)cc2)no1] | <0.1 | <50 |
| | 5-bromo-6-chloro-3-({3-[(2S)-2-(4- | | |
| | chlorophenyl)-2-hydroxyethyl]-1,2,4- | | |
| | oxadiazol-5-yl}methyl)-3,4- | | |
| | dihydropyrimidin-4-one | | |
| 68 | 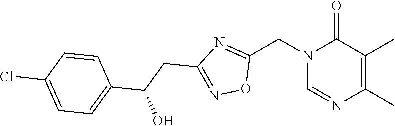 [SMILES: Cc1ncn(Cc2nc(C[C@H](O)c3ccc(Cl)cc3)no2)c(=O)c1C] | <0.3 | * |
| | 3-({3-[(2S)-2-(4-chlorophenyl)-2- | | |
| | hydroxyethyl]-1,2,4-oxadiazol-5- | | |
| | yl}methyl)-5,6-dimethyl-3,4- | | |
| | dihydropyrimidin-4-one | | |
| 69 | 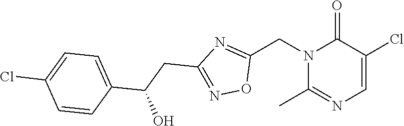 [SMILES: Cc1ncc(Cl)c(=O)n1Cc1nc(C[C@H](O)c2ccc(Cl)cc2)no1] | <1 | * |
| | 5-chloro-3-({3-[(2S)-2-(4-chlorophenyl)- | | |
| | 2-hydroxyethyl]-1,2,4-oxadiazol-5- | | |
| | yl}methyl)-2-methyl-3,4- | | |
| | dihydropyrimidin-4-one | | |
| 70 | 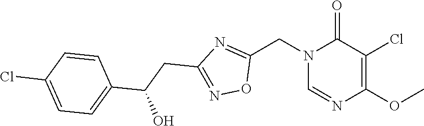 [SMILES: COc1ncn(Cc2nc(C[C@H](O)c3ccc(Cl)cc3)no2)c(=O)c1Cl] | <0.3 | >50 |
| | 5-chloro-3-({3-[(2S)-2-(4-chlorophenyl)- | | |
| | 2-hydroxyethyl]-1,2,4-oxadiazol-5- | | |
| | yl}methyl)-6-methoxy-3,4- | | |
| | dihydropyrimidin-4-one | | |
| 71 | 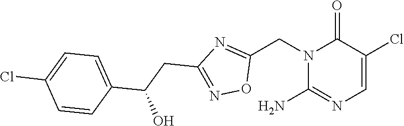 [SMILES: Nc1ncc(Cl)c(=O)n1Cc1nc(C[C@H](O)c2ccc(Cl)cc2)no1] | <0.1 | >50 |
| | 2-amino-5-chloro-3-({3-[(2S)-2-(4- | | |
| | chlorophenyl)-2-hydroxyethyl]-1,2,4- | | |
| | oxadiazol-5-yl}methyl)-3,4- | | |
| | dihydropyrimidin-4-one | | |
| 72 | 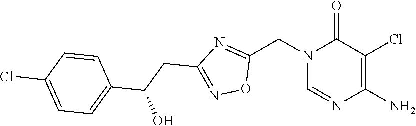 [SMILES: Nc1ncn(Cc2nc(C[C@H](O)c3ccc(Cl)cc3)no2)c(=O)c1Cl] | <0.3 | * |
| | 6-amino-5-chloro-3-({3-[(2S)-2-(4- | | |
| | chlorophenyl)-2-hydroxyethyl]-1,2,4- | | |
| | oxadiazol-5-yl}methyl)-3,4- | | |
| | dihydropyrimidin-4-one | | |
| 73 | 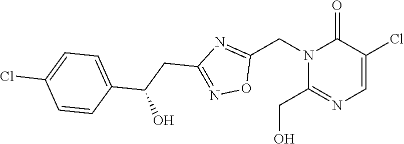 [SMILES: O=c1c(Cl)cnc(CO)n1Cc1nc(C[C@H](O)c2ccc(Cl)cc2)no1] | <0.1 | >50 |
| | 5-chloro-3-({3-[(2S)-2-(4-chlorophenyl)- | | |
| | 2-hydroxyethyl]-1,2,4-oxadiazol-5- | | |
| | yl}methyl)-2-(hydroxymethyl)-3,4- | | |
| | dihydropyrimidin-4-one | | |
| 74 | 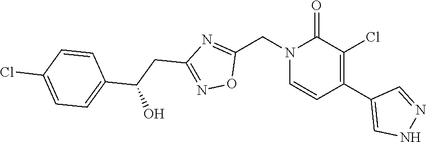 [SMILES: O=c1c(Cl)c(-c2cn[nH]c2)ccn1Cc1nc(C[C@H](O)c2ccc(Cl)cc2)no1] | <1 | * |
| | 3-chloro-1-({3-[(2S)-2-(4-chlorophenyl)- | | |
| | 2-hydroxyethyl]-1,2,4-oxadiazol-5- | | |
| | yl}methyl)-4-(1H-pyrazol-4-yl)-1,2- | | |
| | dihydropyridin-2-one | | |

*not determined (ND)

1. A compound of Formula I, or a pharmaceutically acceptable salt thereof, or a tautomer thereof: 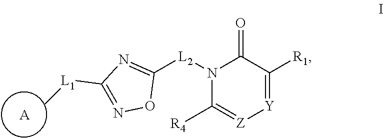
wherein
Y is N or CR₂;
Z is N or CR₃;
R₁ is H, D, halogen, alkyl, cycloalkyl, halogenated alkyl, halogenated cycloalkyl, saturated heterocycle, CN, ORₐ, SRₐ, or NRₐR_{b};
R₂ is H, D, halogen, alkyl, alkenyl, alkynyl, cycloalkyl, halogenated alkyl, halogenated alkenyl, halogenated alkynyl, halogenated cycloalkyl, saturated heterocycle, partially saturated heterocycle, aryl, heteroaryl, alkylaryl, alkylheteroaryl, CN, —C₁₋₄alkyl-CN, ORₐ, SRₐ, NRₐR_{b}, (C═O)NRₐR_{b}, NR_{b}(C═O)Rₐ, (C═O)Rₐ, (C═O)ORₐ, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b};
R₃ is H, D, halogen, alkyl, alkenyl, alkynyl, cycloalkyl, halogenated alkyl, halogenated alkenyl, halogenated alkynyl, halogenated cycloalkyl, saturated heterocycle, partially saturated heterocycle, aryl, heteroaryl, alkylaryl, alkylheteroaryl, CN, —C₁₋₄alkyl-CN, ORₐ, SRₐ, NRₐR_{b}, (C═O)NRₐR_{b}, NR_{b}(C═O)Rₐ, (C═O)Rₐ, (C═O)ORₐ, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b};
R₄ is H, D, halogen, alkyl, cycloalkyl, halogenated alkyl, halogenated cycloalkyl, aryl, heteroaryl, saturated heterocycle, CN, ORₐ, SRₐ, —C₁₋₄alkyl-ORₐ, or NRₐR_{b};
\textcircled{A} is an aryl or heteroaryl each optionally substituted by 1-5 substituents each independently selected from the group consisting of H, D, halogen, alkyl, cycloalkyl, halogenated cycloalkyl, halogenated alkyl, alkenyl, alkynyl, aryl, heteroaryl, CN, ORₐ, SRₐ, NRₐR_{b}, —C₁₋₄alkyl-SRₐ, or —C₁₋₄alkyl-ORₐ;
L₁ is —(CR₅R₆)ₙ—;
each occurrence of R₅ is independently H, D, alkyl, halogen, halogenated alkyl, cycloalkyl, halogenated cycloalkyl, CN, ORₐ, or —C₁₋₄alkyl-ORₐ;
each occurrence of R₆ is independently H, D, alkyl, halogen, halogenated alkyl, cycloalkyl, halogenated cycloalkyl, CN, ORₐ, or —C₁₋₄alkyl-ORₐ;
n is 2 or 3;
L₂ is —CR₇R₈—;
R₇ is H, D, alkyl, halogenated alkyl, cycloalkyl, halogenated cycloalkyl, CN, or —C₁₋₄alkyl-ORₐ;
R₈ is H, D, alkyl, halogenated alkyl, cycloalkyl, halogenated cycloalkyl, CN, or —C₁₋₄alkyl-ORₐ;
each occurrence of Rₐ and R_{b} are independently H, alkyl, (C═O)Rₓ, (C═O)N(Rₓ)₂, SO₂Rₓ, NRₓ(C═O)NRₓ₂, cycloalkyl, halogenated alkyl, heteroalkyl, halogenated heteroalkyl, halogenated cycloalkyl, saturated heterocycle comprising 1-3 heteroatoms each selected from the group consisting of N, O, and S, aryl, or heteroaryl; or alternatively Rₐ and R_{b} together with the carbon or nitrogen atom that they are connected to form a cycloalkyl or saturated heterocycle comprising the nitrogen atom and 0-3 additional heteroatoms each selected from the group consisting of N, O, and S;
the alkyl, alkenyl, alkynyl, cycloalkyl, saturated heterocycle, partially saturated heterocycle, aryl, heteroaryl, alkylaryl, and alkylheteroaryl in R₁, R₂, R₃, R₄, R₅, R₆, R₇, R₈, Rₐ, or R_{b}, where applicable, are optionally substituted by 1-4 substituents each independently selected from the group consisting of alkyl, cycloalkyl, halogenated cycloalkyl, halogenated alkyl, halogen, CN, ORₓ, —(CH₂)₁₋₂ORₓ, N(Rₓ)₂, —(CH₂)₁₋₂N(Rₓ)₂, (C═O)Rₓ, (C═O)N(Rₓ)₂, NRₓ(C═O)Rₓ, and oxo where valence permits; and
each occurrence of Rₓ is independently H, D, alkyl, or optionally substituted heterocycle; or alternatively the two Rₓ groups together with the nitrogen atom that they are connected to form a heterocycle optionally substituted by alkyl and comprising the nitrogen atom and 0-3 additional heteroatoms each selected from the group consisting of N, O, and S;
provided that when Z is N, R₄ is not OH.

2. The compound of claim 1, wherein Y is CR₂.

3. The compound of claim 1, wherein Y is N.

4. The compound of claim 1, wherein Z is CR₃.

5. The compound of claim 1, wherein Z is N.

6. The compound of claim 1, wherein n is 2.

7. The compound of claim 1, wherein each occurrence of R₅ is independently cycloalkyl, halogenated cycloalkyl, —C₁₋₄alkyl-ORₐ, or CN.

8. The compound of claim 1, wherein each occurrence of R₅ is independently H, D, alkyl, halogen, ORₐ, or fluorinated alkyl.

9. The compound of claim 8, wherein each occurrence of R₅ independently H, D, CH₃, CH₂CH₃, OH, F, Cl, or Br.

10. The compound of claim 1, wherein each occurrence of R₆ is independently cycloalkyl, halogenated cycloalkyl, —C₁₋₄alkyl-ORₐ, or CN.

11. The compound of claim 1, wherein each occurrence of R₆ is independently H, D, alkyl, halogen, ORₐ, or fluorinated alkyl.

12. The compound of claim 11, wherein each occurrence of R₆ independently H, D, CH₃, CH₂CH₃, OH, F, Cl, or Br.

13. The compound of claim 1, wherein L₁ is selected from the group consisting of —CH₂—CH₂—, —CH(CH₃)—CH₂—, —CH₂—CH(CH₃)—, —CH₂—C(CH₃)₂—, —CH(OH)—CH₂—, —CH₂—CH(OH)—,

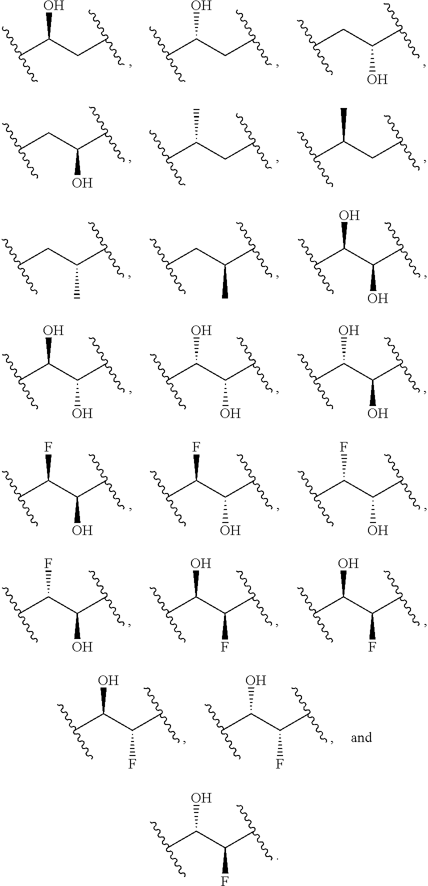

14. The compound of claim 1, wherein L₁ is selected from the group consisting of —CH₂—CH₂—,

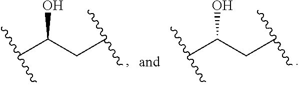 [SMILES: *C[C@@H](*)O ; *C[C@H](*)O]

15. The compound of claim 1, wherein the compound has the structure of Formula Ia, Ib, or Ic: 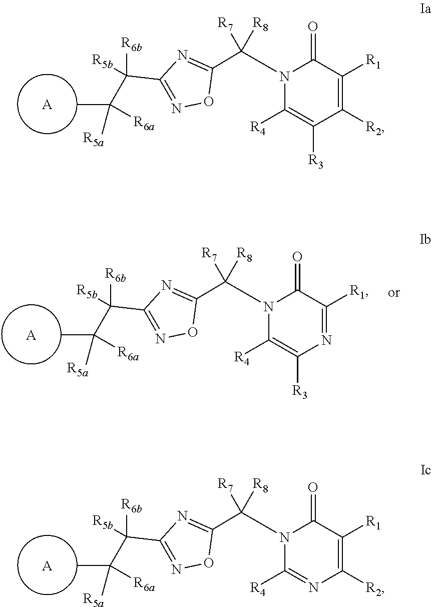
wherein
each occurrence of R₅ₐ is independently H, D, alkyl, halogen, ORₐ, or fluorinated alkyl;
each occurrence of R_{5b} is independently H, D, alkyl, halogen, ORₐ, or fluorinated alkyl;
each occurrence of R₆ₐ is independently H, D, alkyl, halogen, ORₐ, or fluorinated alkyl; and
each occurrence of R_{6b} is independently H, D, alkyl, halogen, ORₐ, or fluorinated alkyl.

16. The compound of claim 1, wherein R₇ is cycloalkyl, halogenated cycloalkyl, CN, or —C₁₋₄alkyl-ORₐ.

17. The compound of claim 1, wherein R₇ is H, D, alkyl, or fluorinated alkyl.

18. The compound of claim 17, wherein R₇ is H, D, CH₃, or CH₂CH₃.

19. The compound of claim 1, wherein R₈ is cycloalkyl, halogenated cycloalkyl, CN, or —C₁₋₄alkyl-ORₐ.

20. The compound of claim 1, wherein R₈ is H, D, alkyl, or fluorinated alkyl.

21. The compound of claim 20, wherein R₈ is H, CH₃, or CH₂CH₃.

22. The compound of claim 1, wherein L₂ is selected from the group consisting of —CH₂—, —CH(CH₃)—, —C(CH₃)₂—, and —CH(CH₂CH₃).

23. The compound of claim 1, wherein L₂ is —CH₂—.

24. The compound of claim 1, wherein \textcircled{A} is phenyl which is optionally substituted with by 1-5 substituents each independently selected from the group consisting of H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, and —C₁₋₄alkyl-ORₐ.

25. The compound of claim 1, wherein the compound has the structure of Formula IIa, IIb, or IIc: 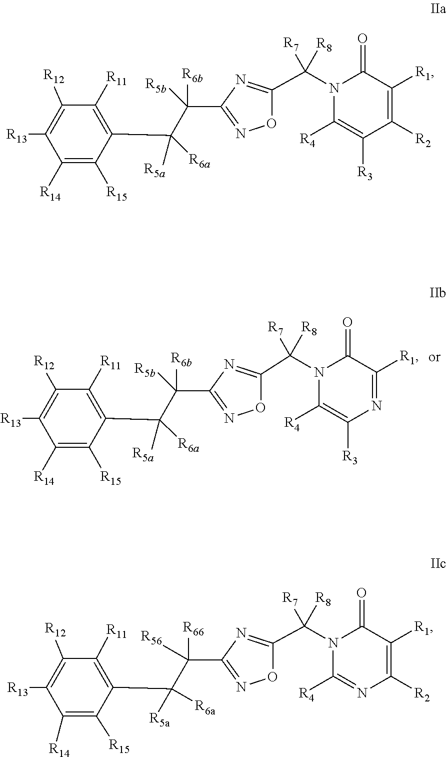
wherein
each occurrence of R₅ₐ is independently H, D, alkyl, halogen, ORₐ, or fluorinated alkyl;
each occurrence of R_{5b} is independently H, D, alkyl, halogen, ORₐ, or fluorinated alkyl;
each occurrence of R₆ₐ is independently H, D, alkyl, halogen, ORₐ, or fluorinated alkyl;
each occurrence of R_{6b} is independently H, D, alkyl, halogen, ORₐ, or fluorinated alkyl;
each occurrence of R₁ is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ;
each occurrence of R₁₂ is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ;
each occurrence of R₁₃ is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ;
each occurrence of R₁₄ is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ; and
each occurrence of R₁₅ is independently H, D, halogen, alkyl, alkenyl, alkynyl, cycloalkyl, halogenated cycloalkyl, halogenated alkyl, aryl, heteroaryl, CN, ORₐ, SRₐ, NRₐR_{b}, —C₁₋₄alkyl-SRₐ, or —C₁₋₄alkyl-ORₐ.

26. The compound of claim 25, wherein R₁₁, R₁₂, R₁₄, and R₁₅ are H; and R₁₃ is H, D, halogen, alkyl, alkenyl, alkynyl, cycloalkyl, CN, CF₃, ORₐ, SRₐ, NRₐR_{b}, or —C₁₋₄alkyl-ORₐ.

27. The compound of claim 26, wherein R₁₃ is CH₃, CH₂CH₃, OH, F, Cl, Br, OCH₃, CH₂OCH₃, CF₃, CN, C≡CH, or

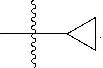 [SMILES: *C1CC1]

28. The compound of claim 1, wherein \textcircled{A} is selected from the group consisting of

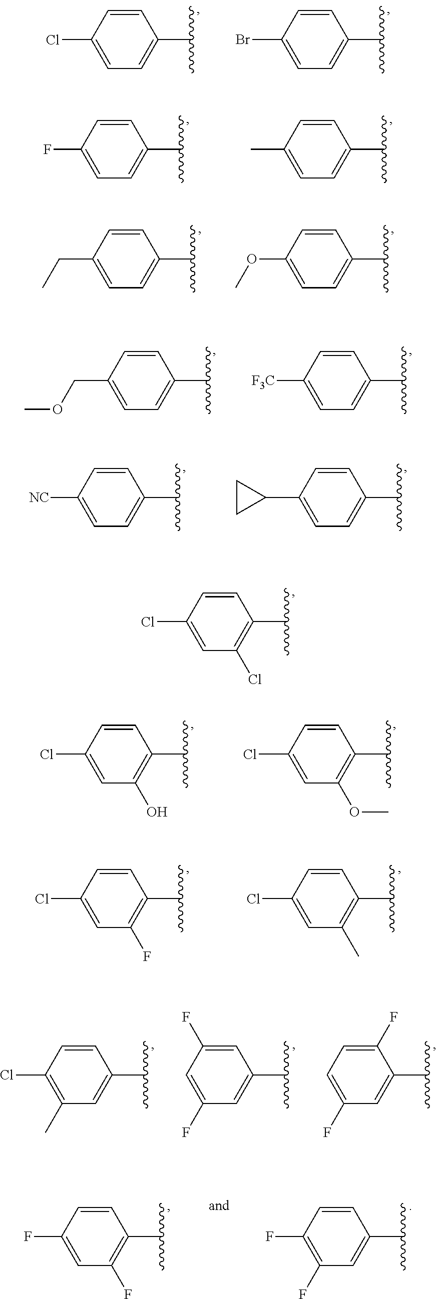

29. The compound of claim 1, wherein \textcircled{A} is a 5- or 6-membered heteroaryl which is optionally substituted with by 1-4 substituents each independently selected from the group consisting of H, halogen, alkyl, cycloalkyl, halogenated cycloalkyl, halogenated alkyl, aryl, heteroaryl, CN, ORₐ, SRₐ, NRₐR_{b}, and —C₁₋₄alkyl-ORₐ.

30. The compound of claim 29, wherein \textcircled{A} is selected from the group consisting of

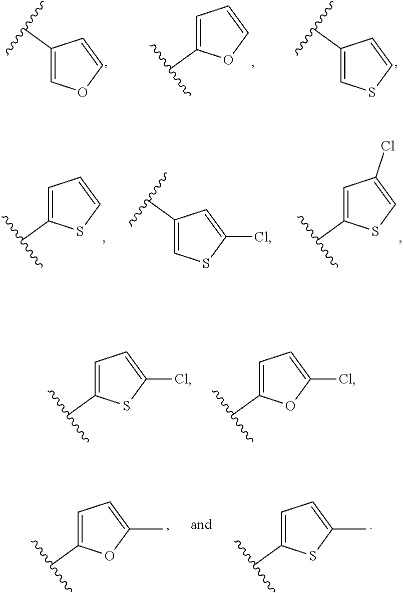

31. The compound of claim 1, wherein R₁ is cycloalkyl, halogenated alkyl, or halogenated cycloalkyl.

32. The compound of claim 1, wherein R₁ is H, D, halogen, alkyl, CN, CF₃, ORₐ, SRₐ, or NRₐR_{b}.

33. The compound of claim 1, wherein R₁ is selected from the group consisting of H, D, CH₃, CH₂CH₃, OH, F, Cl, Br, OCH₃, CF₃, CN, NH₂, NHCH₃, N(CH₃)₂, and

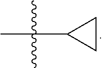 [SMILES: *C1CC1]

34. The compound of claim 1, wherein R₂ is H, D, halogen, CN, CF₃, ORₐ, SRₐ, NRₐR_{b}, (C═O)NRₐR_{b}, NR_{b}(C═O)Rₐ, (C═O)Rₐ, (C═O)ORₐ, —C₁₋₄alkyl-CN, —C₁₋₄alkyl-ORₐ, —C₁₋₄alkyl-SRₐ, —C₁₋₄alkyl-NRₐR_{b}, —C₁₋₄alkyl-COORₐ, —C₁₋₄alkyl-CONRₐR_{b}, —C₁₋₄alkyl-NRₐCOR_{b}, O—C₁₋₄alkyl-Rₐ, or NRₐ—C₁₋₄alkyl-R_{b}.

35. The compound of claim 1, wherein R₂ is saturated heterocycle, partially saturated heterocycle, or heteroaryl, each optionally substituted with 1-3 substituents selected from the group consisting of halogen, alkyl, CN, ORₓ, —(CH₂)₁₋₂ORₓ, N(Rₓ)₂, —(CH₂)₁₋₂N(Rₓ)₂, (C═O)Rₓ, (C═O)N(Rₓ)₂, NRₓ(C═O)Rₓ, and oxo where valence permits.

36. The compound of claim 1, wherein R₂ is alkyl, alkenyl, or alkynyl, each optionally substituted with 1-3 substituents selected from the group consisting of halogen, CN, ORₓ, —(CH₂)₁₋₂ORₓ, N(Rₓ)₂, —(CH₂)₁₋₂N(Rₓ)₂, (C═O)Rₓ, (C═O)N(Rₓ)₂, NRₓ(C═O)Rₓ, and oxo where valence permits.

37. The compound of claim 1, wherein R₂ is cycloalkyl, aryl, or alkylaryl, alkylheteroaryl.

38. The compound of claim 1, wherein R₂ is selected from the group consisting of H, D, CH₃, CH₂CH₃, OH, F, Cl, Br, I, OCH₃, CF₃, CN, NH₂, NHCH₃, N(CH₃)₂, CH═CH₂,

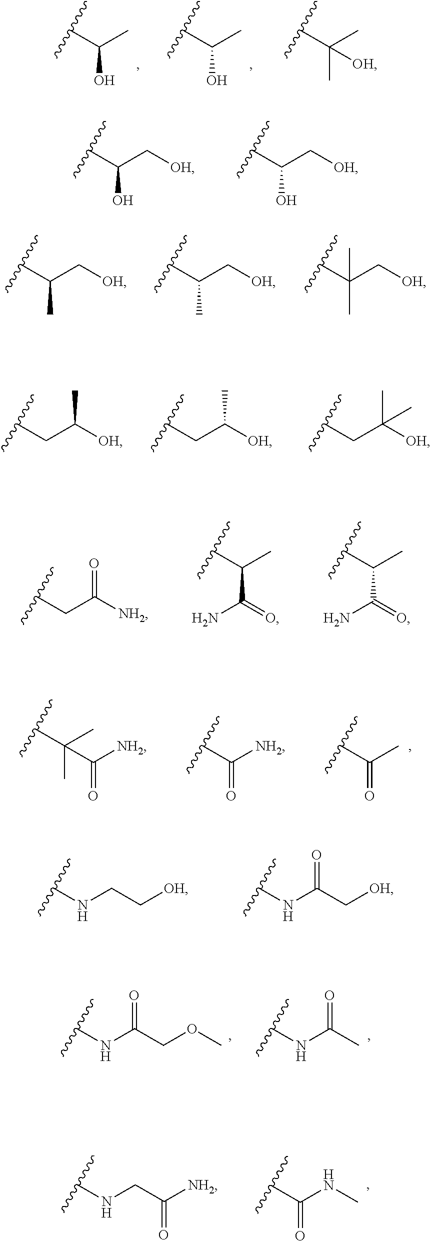

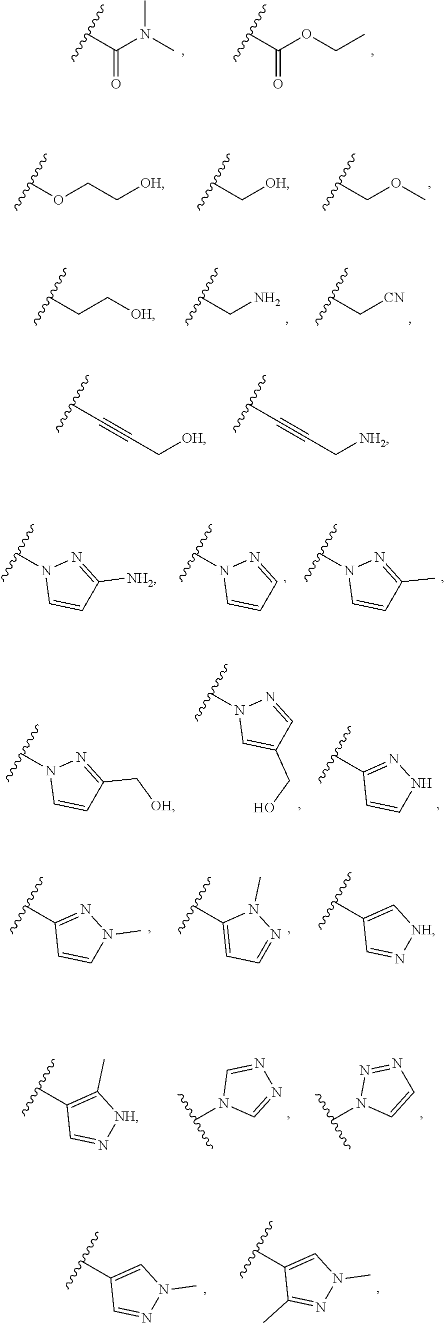

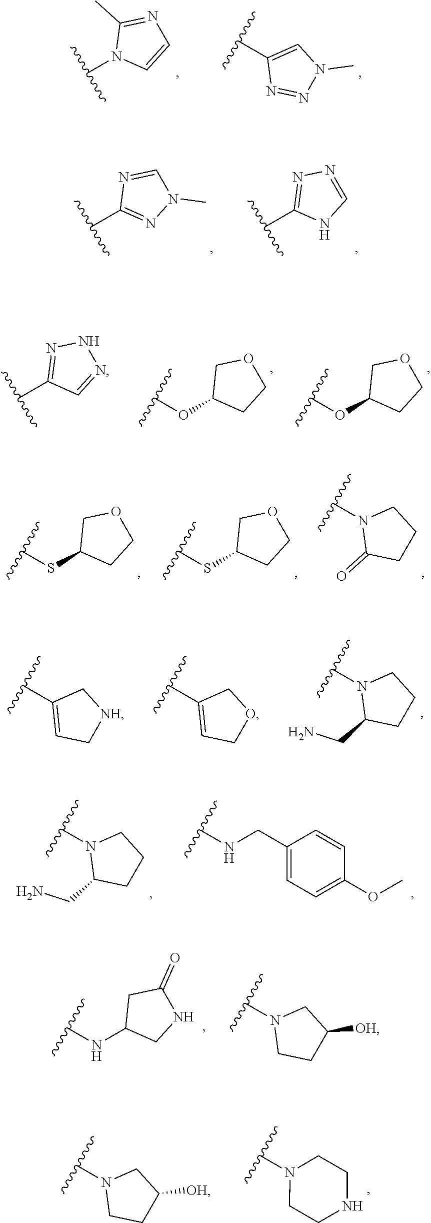

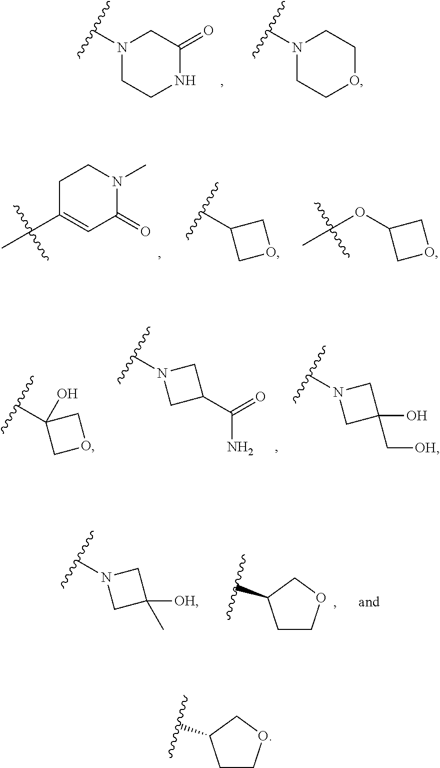

39. The compound of claim 1, wherein R₃ is H, D, halogen, alkyl, halogenated alkyl, heteroaryl, or CN.

40. The compound of claim 1, wherein R₃ is ORₐ, SRₐ, NRₐR_{b}, (C═O)NRₐR_{b}, —C₁₋₄alkyl-CN, —C₁₋₄alkyl-ORₐ, —C₁₋₄alkyl-SRₐ, —C₁₋₄alkyl-NRₐR_{b}, or —C₁₋₄alkyl-CONRₐR_{b}.

41. The compound of claim 1, wherein R₃ is alkenyl, alkynyl, cycloalkyl, saturated heterocycle, partially saturated heterocycle, aryl, alkylaryl, alkylheteroaryl, NR_{b}(C═O)Rₐ, (C═O)Rₐ, (C═O)ORₐ, —C₁₋₄alkyl-COORₐ, —C₁₋₄alkyl-NRₐCOR_{b}, O—C₁₋₄alkyl-Rₐ, or NRₐ—C₁₋₄alkyl-R_{b}.

42. The compound of claim 1, wherein R₃ is selected from the group consisting of H, D, CH₃, CH₂CH₃, OH, F, Cl, Br, OCH₃, CF₃, CN, CH₂CN, CH═CH₂, NH₂, NHCH₃, N(CH₃)₂,

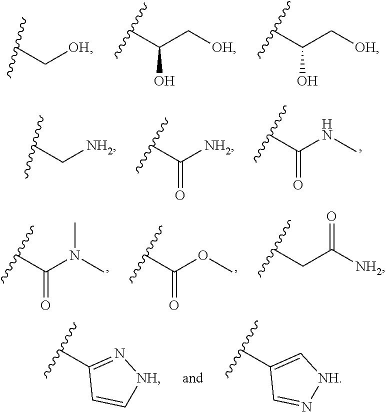

43. The compound of claim 1, wherein R₄ is cycloalkyl, halogenated alkyl, or halogenated cycloalkyl.

44. The compound of claim 1, wherein R₄ is H, D, halogen, alkyl, CN, CF₃, ORₐ, SRₐ, —C₁₋₄alkyl-ORₐ, or NRₐR_{b}.

45. The compound of claim 1, wherein R₄ is selected from the group consisting of H, D, CH₃, CH₂CH₃, OH, F, Cl, Br, OCH₃, CF₃, CN, NH₂, NHCH₃, N(CH₃)₂, CH₂OH, H, and

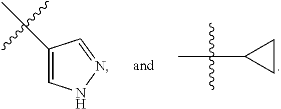

46. The compound of claim 1, wherein at least one occurrence of Rₐ or R_{b} is independently H, alkyl, cycloalkyl, saturated heterocycle, aryl, or heteroaryl.

47. The compound of claim 46, wherein at least one occurrence of Rₐ or R_{b} is independently H, D, Me, Et, Pr, CH₂CH₂OH, phenyl, or a heterocycle selected from the group consisting of 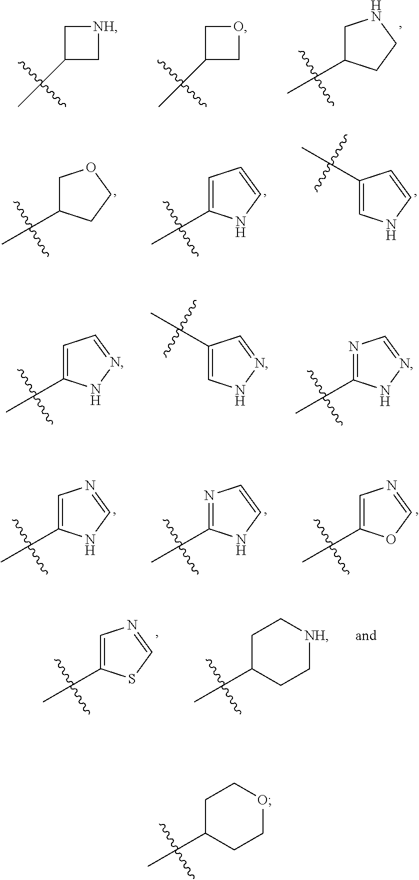
wherein the heterocycle is optionally substituted by alkyl, OH, oxo, or (C═O)C₁₋₄alkyl where valence permits.

48. The compound of claim 47, wherein at least one occurrence of Rₐ or R_{b} is H, Me, phenyl,

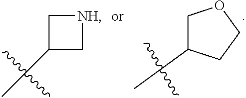 [SMILES: *C1CNC1 ; *C1CCOC1]

49. The compound of claim 1, wherein Rₐ and R_{b} together with the nitrogen atom that they are connected to form an optionally substituted heterocycle comprising the nitrogen atom and 0-3 additional heteroatoms each selected from the group consisting of N, O, and S.

50. The compound of claim 1, wherein each occurrence of Rₓ is independently H, alkyl, or heterocycle optionally substituted by alkyl, halogen, or OH.

51. The compound of claim 50, wherein each occurrence of Rₓ is independently H or alkyl.

52. The compound of claim 51, wherein each occurrence of Rₓ is independently H or Me.

53. The compound of claim 1, wherein the compound is selected from the group consisting of compounds in Examples 2-5 and Tables 1-5.

54. A pharmaceutical composition comprising at least one compound according to claim 1 or a pharmaceutically acceptable salt thereof and a pharmaceutically acceptable carrier or diluent.

55. A method of treating a condition in a mammalian species in need thereof, comprising administering to the mammalian species a therapeutically effective amount of at least one compound according to claim 1 or a pharmaceutically acceptable salt thereof, wherein the condition is selected from the group consisting of pain, a skin disorder, a respiratory disease, a fibrotic disease, an inner ear disorder, fever or another disorder of thermoregulation, a urinary tract or bladder disorder, an autoimmune disease, ischemia, a central nervous system (CNS) disorder, an inflammatory disorder, a gastroenterological disorder, and a cardiovascular disorder.

56. The method of claim 55, wherein the pain is acute pain, chronic pain, complex regional pain syndrome, inflammatory pain, neuropathic pain, postoperative pain, rheumatoid arthritic pain, osteoarthritis pain, back pain, visceral pain, cancer pain, algesia, neuralgia, migraine, neuropathies, diabetic neuropathy, sciatica, HIV-related neuropathy, pos-herpetic neuralgia, fibromyalgia, nerve injury, post stock pain, or tooth and tooth injury-related pain.

57. The method of claim 55, wherein the urinary tract or bladder disorder is pelvic hypersensitivity, urinary incontinence, cystitis, bladder instability, or bladder outlet obstruction.

58. The method of claim 55, wherein the skin disorder is burns, psoriasis, eczema, or pruritus.

59. The method of claim 55, wherein the skin disorder is atopic dermatitis or psoriasis-induced itching.

60. The method of claim 55, wherein the respiratory disease is an inflammatory airway disease, airway hyperresponsiveness, an idiopathic lung disease, chronic obstructive pulmonary disease, asthma, chronic asthma, tracheobronchial or diaphragmatic dysfunction, cough, or chronic cough.

61. The method of claim 55, wherein the ischemia is CNS hypoxia or a disorder associated with reduced blood flow to CNS.

62. The method of claim 55, wherein the autoimmune disease is rheumatoid arthritis or multiple sclerosis.

63. The method of claim 55, wherein the central nervous system disorder is associated with neurodegeneration.

64. The method of claim 55, wherein the gastroenterological disorder is an inflammatory bowel disease, esophagitis, gastroesophageal reflux disorder, irritable bowel syndrome, emesis, or stomach duodenal ulcer.

65. The method of claim 55, wherein the cardiovascular disorder is stroke, myocardial infarction, atherosclerosis, or cardiac hypertrophy.

66. The method of claim 55, wherein the mammalian species is human.

67. A method of inhibiting transient receptor potential ankyrin 1 (TRPA1) in a mammalian species in need thereof, comprising administering to the mammalian species a therapeutically effective amount of at least one compound according to claim 1 or a pharmaceutically acceptable salt thereof.

68. The method of claim 67, wherein the mammalian species is human.
